SEQUENCE LISTING (28 sequences)

SEQ ID NO: 1 - DNA, 1797 bp, Homo sapiens
  misc_feature: NK45-1 DNA
atggccttaccagtgaccgccttgctcctgccgctggccttgctgctccacgccgccaggccgttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgc

SEQ ID NO: 2 - protein, 599 aa, Homo sapiens
  MISC_FEATURE: NK45-5 AA
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 3 - DNA, 2150 bp, Homo sapiens
  misc_feature: DNA NK19H-No Flag-1
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctattattgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggattggcgtgatttggggcagcgaaaccacctattataacagcgcgctgaaaagccgcctgaccattagcaaagataacagcaaaaaccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 4 - protein, 706 aa, Homo sapiens
  MISC_FEATURE: AA NK19H-No Flag-1
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 5 - DNA, 2150 bp, Homo sapiens
  misc_feature: DNA NK19H-No Flag-2
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggattggcgtgatttggggcagcgaaaccacctattataacagcgcgctgaaaagccgcctgaccattagcaaagataacagcaaaaaccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 6 - protein, 706 aa, Homo sapiens
  MISC_FEATURE: AA NK19H-No Flag-2
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 7 - DNA, 2150 bp, Homo sapiens
  misc_feature: DNA NK19H-No Flag-3
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccggatggcaccgtgaaactgctgatttatcataccagccgcctgcatagcggcgtgccgagccgctttagcggcagcggcagcggcaccgattataccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggattggcgtgatttggggcagcgaaaccacctattataacagcgcgctgaaaagccgcctgaccattagcaaagataacagcaaaaaccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 8 - protein, 706 aa, Homo sapiens
  MISC_FEATURE: AA NK19H-No Flag-3
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 9 - DNA, 2150 bp, Homo sapiens
  misc_feature: DNA NK19H-No Flag-4
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctattattgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggctgggcgtgatttggggcagcgaaaccacctattataacagcgcgctgaaaagccgcctgaccattagcaaagataacagcaaaagccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 10 - protein, 706 aa, Homo sapiens
  MISC_FEATURE: AA NK19H-No Flag-4
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 11 - DNA, 2153 bp, Homo sapiens
  misc_feature: DNA NK19H-No Flag-5
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggagtcaggacctggcctggtgaaaccctcacagactctgtccctgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcagatctagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 12 - protein, 707 aa, Homo sapiens
  MISC_FEATURE: AA NK19H-No Flag-5
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 13 - DNA, 2150 bp, Homo sapiens
  misc_feature: DNA NK19H-No Flag-6
gaattcgccgccaccatggccttaccagtgaccgccttgctcctgccgctggccttgctgctccacgccgccaggccggacatccagatgacacagagcccgtcctccctgtctgcctctgtgggagacagagtcaccatcacctgcagggcaagtcaggacattagtaaatatttaaattggtatcagcagaaaccagacggaactgttaaactcctgatctaccatacatcaagattacactcaggagtcccatcaaggttcagtggcagtgggtctggaacagattacaccctcaccattagcagcctgcaaccggaagatattgccacttacttctgccaacagggtaatacgcttccgtacacgttcggaggggggaccaagctggagatcacaggtggcggtggctcgggcggtggtgggtcgggtggcggcggatctcaggtgcagctgcaggagtcaggacctggcctggtgaaaccctcacagactctgtccctgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 14 - protein, 706 aa, Homo sapiens
  MISC_FEATURE: AA NK19H-No Flag-6
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 15 - DNA, 2150 bp, Homo sapiens
  misc_feature: DNA NK19H-No Flag-7
gaattcgccgccaccatggccttaccagtgaccgccttgctcctgccgctggccttgctgctccacgccgccaggccggacatccagatgacacagagcccgtcctccctgtctgcctctgtgggagacagagtcaccatcacctgcagggcaagtcaggacattagtaaatatttaaattggtatcagcagaaaccaggtggaactgttaaactcctgatctaccatacatcaagattacactcaggagtcccatcaaggttcagtggcagtgggtctggaacagatttcaccctcaccattagcagcctgcaaccggaagatattgccacttactactgccaacagggtaatacgcttccgtacacgttcggaggggggaccaagctggagatcacaggtggcggtggctcgggcggtggtgggtcgggtggcggcggatctcaggtgcagctgcaggagtcaggacctggcctggtgaaaccctcacagactctgtccgtgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 16 - protein, 706 aa, Homo sapiens
  MISC_FEATURE: AA NK19H-No Flag-7
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 17 - DNA, 2150 bp, Homo sapiens
  misc_feature: DNA NK19H-No Flag-8
gaattcgccgccaccatggccttaccagtgaccgccttgctcctgccgctggccttgctgctccacgccgccaggccggacatccagatgacacagagcccgtcctccctgtctgcctctgtgggagacagagtcaccatcacctgcagggcaagtcaggacattagtaaatatttaaattggtatcagcagaaaccaggtggaactgttaaactcctgatctaccatacatcaagattacactcaggagtcccatcaaggttcagtggcagtgggtctggaacagatttcaccctcaccattagcagcctgcaaccggaagatattgccacttacttctgccaacagggtaatacgcttccgtacacgttcggaggggggaccaagctggagatcacaggtggcggtggctcgggcggtggtgggtcgggtggcggcggatctcaggtgcagctgcaggagtcaggacctggcctggtgaaaccctcacagactctgtccgtgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 18 - protein, 706 aa, Homo sapiens
  MISC_FEATURE: AA NK19H-No Flag-8
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 19 - DNA, 2150 bp, Homo sapiens
  misc_feature: DNA NK19H-No Flag-9
gaattcgccgccaccatggccttaccagtgaccgccttgctcctgccgctggccttgctgctccacgccgccaggccggacatccagatgacacagagcccgtcctccctgtctgcctctgtgggagacagagtcaccatcacctgcagggcaagtcaggacattagtaaatatttaaattggtatcagcagaaaccagacggaactgttaaactcctgatctaccatacatcaagattacactcaggagtcccatcaaggttcagtggcagtgggtctggaacagattacaccctcaccattagcagcctgcaaccggaagatattgccacttacttctgccaacagggtaatacgcttccgtacacgttcggaggggggaccaagctggagatcacaggtggcggtggctcgggcggtggtgggtcgggtggcggcggatctcaggtgcagctgcaggagtcaggacctggcctggtgaaaccctcacagactctgtccgtgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 20 - protein, 706 aa, Homo sapiens
  MISC_FEATURE: AA NK19H-No Flag-9
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 21 - DNA, 2150 bp, Homo sapiens
  misc_feature: DNA NK19H-No Flag-10
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctattattgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcgtgacctgcaccgtgagcggcgtgagcctgccggattatggcgtgagctggattcgccagccgccgcgcaaaggcctggaatggctgggcgtgatttggggcagcgaaaccacctattataacagcgcgctgaaaagccgcctgaccattagcaaagataacagcaaaagccaggtgagcctgaaaatgagcagcgtgaccgcggcggataccgcgatttattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 22 - protein, 706 aa, Homo sapiens
  MISC_FEATURE: AA NK19H-No Flag-10
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 23 - DNA, 2150 bp, Homo sapiens
  misc_feature: DNA NK19H-No Flag-11
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcgtgacctgcaccgtgagcggcgtgagcctgccggattatggcgtgagctggattcgccagccgccgcgcaaaggcctggaatggctgggcgtgatttggggcagcgaaaccacctattataacagcgcgctgaaaagccgcctgaccattagcaaagataacagcaaaagccaggtgagcctgaaaatgagcagcgtgaccgcggcggataccgcgatttattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 24 - protein, 706 aa, Homo sapiens
  MISC_FEATURE: AA NK19H-No Flag-11
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 25 - DNA, 2150 bp, Homo sapiens
  misc_feature: DNA NK19H-No Flag-12
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccggatggcaccgtgaaactgctgatttatcataccagccgcctgcatagcggcgtgccgagccgctttagcggcagcggcagcggcaccgattataccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcgtgacctgcaccgtgagcggcgtgagcctgccggattatggcgtgagctggattcgccagccgccgcgcaaaggcctggaatggctgggcgtgatttggggcagcgaaaccacctattataacagcgcgctgaaaagccgcctgaccattagcaaagataacagcaaaagccaggtgagcctgaaaatgagcagcgtgaccgcggcggataccgcgatttattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 26 - protein, 706 aa, Homo sapiens
  MISC_FEATURE: AA NK19H-No Flag-12
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 27 - DNA, 2210 bp, Homo sapiens
  misc_feature: DNA NK19-1
ggatccgaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 28 - protein, 724 aa, Homo sapiens
  MISC_FEATURE: AA NK19-1
MALPVTALLLPLALLLHAARPDYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

(19) United States
(12) Patent Application Publication
(10) Pub. No.: US 2023/0265390 A1
(43) Pub. Date: Aug. 24, 2023

(54) ENHANCED EXPANSION AND CYTOTOXICITY OF ENGINEERED NATURAL KILLER CELLS AND USES THEREOF

(71) Applicant: Nkarta, Inc., South San Francisco, CA (US)

(72) Inventors: James Barnaby Trager, Albany, CA (US); Alexandra Leida Liana Lazetic, San Jose, CA (US); Katherine Jamboretz, San Francisco, CA (US); Muhammad Nafeesur Rahman, Redwood City, CA (US)

(21) Appl. No.: 18/024,012

(22) PCT Filed: Aug. 31, 2021
(86) PCT No.: PCT/US2021/071330

§ 371 Date: Feb. 28, 2023

Related U.S. Application Data
(60) Provisional application No. 63/073,671, filed on Sep. 2, 2020.

(51) Int. Cl. C12N 5/0783; C07K 14/705; A61K 35/17

(52) U.S. Cl. CPC C12N 5/0646; C07K 14/70503; C07K 14/7056; A61K 35/17; C12N 2501/2302; C12N 2501/2312; C12N 2501/2318; C12N 2502/30

(57) ABSTRACT

Several embodiments disclosed herein relate to methods and compositions for enhanced expansion of NK cells in culture. In several embodiments, the methods utilize one or more soluble interleukins as culture media supplements at one or more time points during expansion of the NK cell, or other immune cell, the expansion employing a feeder cell population.

### RELATED CASES

This application claims priority to U.S. Provisional Patent Application No. 63/073,671, filed Sep. 2, 2020, the entire contents of which is incorporated by reference herein.

### FIELD

Some embodiments of the methods and compositions disclosed herein relate to enhanced expansion and/or enhanced cytotoxicity of engineered immune cells, such as Natural Killer (NK) cells and/or T cells.

### BACKGROUND

The use of engineered cells for cellular immunotherapy allows for treatment of cancers or other diseases by leveraging various aspects of the immune system to target and destroy diseased or damaged cells. Such therapies require engineered cells in numbers sufficient for therapeutically relevant doses.

### INCORPORATION BY REFERENCE OF MATERIAL IN ASCII TEXT FILE

This application incorporates by reference the Sequence Listing contained in the following ASCII text file being submitted concurrently herewith: File name: NKT064WO_ST25.txt; created Aug. 31, 2021, 126186 bytes in size.

### SUMMARY

In several embodiments, there are provided various methods for enhancing the expansion of immune cells for use in cellular immunotherapy. For example, in several embodiments, there is provided a method in which immune cells are repeatedly co-cultured with a feeder cell line in a media supplemented with stimulatory cytokines. In several embodiments, fresh (e.g., un-used) media and feeder cells are introduced at the inception of each repetition of co-culturing. In several embodiments, each co-culturing starts with a particular ratio of cells to be expanded to feeder cells. In several embodiments the repeated co-culturing leads to significantly enhanced expansion of the NK cells (e.g., greater than 1 million-fold, according to several embodiments). In several embodiments, the immune cells are NK cells. In several embodiments, the expanded NK cells are unexpectedly amenable to cellular engineering, such as engineering the cells to express a chimeric receptor (for example, for use in cancer immunotherapy). In several embodiments, the NK cells (or other immune cells) repeatedly co-cultured with feeder cells express such chimeric receptors more robustly than NK cells not subject to the multi-pulse co-culturing. Further, in several embodiments, the engineered NK cells exhibit an unexpectedly enhanced cytotoxicity.

In several embodiments, there is provided a method for enhancing the expansion of natural killer cells for use in immunotherapy, comprising co-culturing, in a culture media, a population of natural killer (NK) cells with a first population of feeder cells for a first period of time, wherein the first feeder cell population comprises cells engineered to express 4-1 BBL and membrane-bound interleukin-15 (mbIL15), wherein the population of NK cells is smaller than the population of feeder cells, wherein the culture media comprises interleukin 2 (IL2), interleukin 12 (IL12), and interleukin 18 (IL18), wherein the co-culturing for the first period of time results in an expanded population of NK cells, followed by separating, after the first period of time, at least a portion of the expanded population of NK cells from the feeder cells, and co-culturing, in fresh culture media, the at least a portion of the expanded population of NK cells with a second population of the feeder cells for a second period of time, wherein the population of NK cells is smaller than the population of feeder cells, wherein the culture media comprises interleukin 2 (IL2), interleukin 12 (IL12), and interleukin 18 (IL18), and wherein the co-culturing for the second period of time results in a further expanded population of NK cells. In several embodiments, the methods further comprise optionally repeating the separating and co-culturing steps at least one additional time using fresh culture media comprising IL2, IL12, and IL18, thereby resulting in additional expansion of the further expanded population of NK cells.

In several embodiments, the repeated co-culturing of the expanded NK cells with an additional population of the feeder cells and fresh media results in enhanced NK cell expansion as compared to expanding NK cells with the feeder cells in the absence of the repeated co-culturing.

In several embodiments, the IL2 is present in the media at a concentration between about 10 units/mL and about 100 units/mL. In some embodiments, the IL2 is present in the media at a concentration of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 units/mL or any concentration within a range defined by any two of the aforementioned concentrations. In some embodiments, the IL12 is present in the media at a concentration between about 10 ng/mL and about 100 ng/mL. In some embodiments, the IL12 is present in the media at a concentration of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng/mL or any concentration within a range defined by any two of the aforementioned concentrations. In some embodiments, the IL18 is present in the media at a concentration between about 0.01 ng/mL and about 30 ng/mL. In some embodiments, the IL18 is present in the media at a concentration of 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ng/mL or any concentration within a range defined by any two of the aforementioned concentrations. In some embodiments, the first and the second period of time are about 7 days. In some embodiments, the co-culturing is repeated at least three times. In some embodiments, the IL2 is present in the media at a concentration between about 10 units/mL and about 100 units/mL, the IL12 is present in the media at a concentration between about 10 ng/mL and about 100 ng/mL, and the IL18 is present in the media at a concentration between about 0.01 ng/mL and about 30 ng/mL. In some embodiments, the IL2 is present in the media at a concentration between about 10 units/mL and about 100 units/mL, the IL12 is present in the media at a concentration between about 10 ng/mL and about 100 ng/mL, and the IL18 is present in the media at a concentration between about 0.01 ng/mL and about 30 ng/mL, where the first and the second period of time are about 7 days, and where the co-culturing is repeated at least three time. These concentrations of IL2 in terms of units/mL may be determined according to the WHO International Standard for IL2 (National Institute for Biological Standards and Control [NIBSC] 86/500).

In several embodiments, the IL2 is present in the media at a concentration between about 0.575 ng/mL and about 5.75 ng/mL. In some embodiments, the IL2 is present in the media at a concentration between about 0.5 ng/mL to about 6 ng/mL. In some embodiments, the IL2 is present in the media at a concentration of 0.5, 0.575, 0.6, 0.7, 0.8, 0.9, 1, 1.5, 2, 2.5, 3, 3.5, 4, 4.5, 5, 5.5, 5.75, or 6 ng/mL, or any concentration within a range defined by any two of the aforementioned concentrations. In some embodiments, the IL12 is present in the media at a concentration between about 10 ng/mL and about 100 ng/mL. In some embodiments, the IL12 is present in the media at a concentration of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng/mL, or any concentration within a range defined by any two of the aforementioned concentrations. In some embodiments, the IL18 is present in the media at a concentration between about 0.01 ng/mL and about 30 ng/mL. In some embodiments, the IL18 is present in the media at a concentration of 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ng/mL, or any concentration within a range defined by any two of the aforementioned concentrations. In some embodiments, the first and the second period of time are about 7 days. In some embodiments, the co-culturing is repeated at least three times. In some embodiments, the IL2 is present in the media at a concentration between about 0.575 ng/mL and about 5.75 ng/mL (or between about 0.5 ng/mL to about 6 ng/mL), the IL12 is present in the media at a concentration between about 10 ng/mL and about 100 ng/mL, and the IL18 is present in the media at a concentration between about 0.01 ng/mL and about 30 ng/mL. In some embodiments, the IL2 is present in the media at a concentration between about 0.575 ng/mL and about 5.75 ng/mL (or between about 0.5 ng/mL to about 6 ng/mL), the IL12 is present in the media at a concentration between about 10 ng/mL and about 100 ng/mL, and the IL18 is present in the media at a concentration between about 0.01 ng/mL and about 30 ng/mL, where the first and the second period of time are about 7 days, and where the co-culturing is repeated at least three time.

In several embodiments, the population of NK cells is present in an amount between about 5 and about 25 times less than the population of feeder cells at inception of each co-culturing. In several embodiments, the expanded NK cells are separated from the feeder cells by Fluorescence-Activated Cell Sorting (FACS).

In several embodiments, the feeder cell population comprises K562 cells that express both 4-1BBL and mbIL15. In several embodiments, the repeated co-culturing increases expression of markers of NK cells activation. Additionally, in several embodiments, the repeated co-culturing increases the cytotoxicity and/or persistence of the expanded NK cells.

In several embodiments, the method further comprises contacting the NK cells with a vector encoding a chimeric antigen receptor (CAR). In several embodiments, the CAR is configured to target one or more of CD19, CD123, CD70, BCMA, or a ligand of the natural killer receptor group D (NKG2D).

In some embodiments, the IL2 is present in the media at a concentration of less than about 10, 20, 30, 40, 50, 60, 70, 80, 90, or 100 units/mL, or any concentration within a range defined by any two of the aforementioned concentrations. In some embodiments, the IL2 is present in the media at a concentration of less than about 50 units/mL. In some embodiments, the IL12 is present in the media at a concentration of less than about 10, 20, 30, 40, 50, 60, 70, 80, 90, or 100 ng/mL, or any concentration within a range defined by any two of the aforementioned concentrations. In some embodiments, the IL12 is present in the media at a concentration less than about 30 ng/mL. In some embodiments, the IL18 is present in the media at a concentration of less than about 1, 5, 10, 15, 20, 25, 30, 35, 40, 45, or 50 ng/mL, or any concentration within a range defined by any two of the aforementioned concentrations. In some embodiments, the IL18 is present in the media at a concentration of less than about 10 ng/mL. In several embodiments, the IL2 is present in the media at a concentration of less than about 50 units/mL, the IL12 is present in the media at a concentration less than about 30 ng/mL, and the IL18 is present in the media at a concentration of less than about 10 ng/mL.

In some embodiments, the IL2 is present in the media at a concentration of less than about 1, 2, 3, 4, 5, 6, 7, 8, 9, or 10 mL, or any concentration within a range defined by any two of the aforementioned concentrations. In some embodiments, the IL2 is present in the media at a concentration of less than about 6 ng/mL. In some embodiments, the IL12 is present in the media at a concentration of less than about 10, 20, 30, 40, 50, 60, 70, 80, 90, or 100 ng/mL. In some embodiments, the IL12 is present in the media at a concentration less than about 30 ng/mL. In some embodiments, the IL18 is present in the media at a concentration of less than about 1, 5, 10, 15, 20, 25, 30, 35, 40, 45, or 50 ng/mL. In some embodiments, the IL18 is present in the media at a concentration of less than about 10 ng/mL. In several embodiments, the IL2 is present in the media at a concentration of less than about 6 ng/mL, the IL12 is present in the media at a concentration less than about 30 ng/mL, and the IL18 is present in the media at a concentration of less than about 10 ng/mL.

In several embodiments, the IL2 is present in the media at a concentration between about 20 units/mL and about 50 units/mL. In some embodiments, the IL2 is present in the media at a concentration of about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 units/mL, or any concentration within a range defined by any two of the aforementioned concentrations. In some embodiments, the IL12 is present in the media at a concentration between about 15 ng/mL and about 30 ng/mL. In some embodiments, the IL12 is present in the media at a concentration of 15, 16, 17, 18, 19, 20, 21, 22, 23, 24, 25, 26, 27, 28, 29, or 30 ng/mL, or any concentration within a range defined by any two of the aforementioned concentrations. In some embodiments, the IL18 is present in the media at a concentration of less than about 5 ng/mL. In some embodiments, the IL18 is present in the media at a concentration of less than 0.01, 0.02, 0.03, 0.04, 0.05, 0.06, 0.07, 0.08, 0.09, 0.1, 1, 2, 3, 4, or 5 ng/mL, or any concentration within a range defined by any two of the aforementioned concentrations. In several embodiments, the IL2 is present in the media at a concentration between about 20 units/mL and about 50 units/mL, wherein the IL12 is present in the media at a concentration between about 15 ng/mL and about 30 ng/mL, and wherein the IL18 is present in the media at a concentration of less than about 5 ng/mL.

In several embodiments, the IL2 is present in the media at a concentration between about 1.15 ng/mL and about 2.875 units/mL. In several embodiments, the IL2 is present in the media at a concentration between about 1 ng/mL and about 3 units/mL. In some embodiments, the IL2 is present in the media at a concentration of about 1, 1.15, 1.5, 2, 2.5, 2.875, or 3 ng/mL, or any concentration within a range defined by any two of the aforementioned concentrations. In some embodiments, the IL12 is present in the media at a concentration between about 15 ng/mL and about 30 ng/mL. In some embodiments, the IL12 is present in the media at a concentration of 15, 16, 17, 18, 19, 20, 21, 22, 23, 24, 25, 26, 27, 28, 29, or 30 ng/mL, or any concentration within a range defined by any two of the aforementioned concentrations. In some embodiments, the IL18 is present in the media at a concentration of less than about 5 ng/mL. In some embodiments, the IL18 is present in the media at a concentration of less than 0.01, 0.02, 0.03, 0.04, 0.05, 0.06, 0.07, 0.08, 0.09, 0.1, 1, 2, 3, 4, or 5 ng/mL, or any concentration within a range defined by any two of the aforementioned concentrations. In several embodiments, the IL2 is present in the media at a concentration between about 1.15 ng/mL and about 2.875 ng/mL, wherein the IL12 is present in the media at a concentration between about 15 ng/mL and about 30 ng/mL, and wherein the IL18 is present in the media at a concentration of less than about 5 ng/mL. In several embodiments, the IL2 is present in the media at a concentration between about 1 ng/mL and about 3 ng/mL, wherein the IL12 is present in the media at a concentration between about 15 ng/mL and about 30 ng/mL, and wherein the IL18 is present in the media at a concentration of less than about 5 ng/mL.

Also provided for herein is the use of the NK cells expanded by the methods disclosed herein for the preparation of a medicament for the treatment of cancer. Also provided for herein is the use of the NK cells expanded by the methods disclosed herein for the treatment of cancer.

Provided for herein, in several embodiments, is a population of engineered natural killer cells comprising, an engineered chimeric receptor configured to bind a marker on a target cancer cell and upon binding, induce the NK cell to exert a cytotoxic effect against the target cancer cell, wherein the NK cell was expanded by co-culturing for a first time, in a culture media interleukin 2 (IL2), interleukin 12 (IL12), and interleukin 18 (IL18), a starting population of natural killer (NK) cells with a first population of feeder cells, wherein the first feeder cell population comprises cells engineered to express 4-1 BBL and membrane-bound interleukin-15 (mbIL15), wherein the starting population of NK cells is smaller than the population of feeder cells, wherein the first co-culturing results in an intermediate expanded population of NK cells, separating, after the first co-culturing, at least a portion of the intermediate expanded population of NK cells from the feeder cells, co-culturing for at least as second time, in fresh culture media, at least a portion of the intermediate expanded population of NK cells with a second population of the feeder cells, wherein the portion of the population of NK cells co-cultured with the second population of feeder cells is smaller than the second population of feeder cells, and wherein the at least a second co-culturing results in a further expanded population of NK cells.

In several embodiments, the engineered chimeric receptor is encoded by a sequence at least 95% identical in sequence to SEQ ID NO: 1, 3, 5, 7, 9, 11, 13, 15, 17, 19, 21, 23, 25, or 27. In several embodiments, the engineered chimeric receptor has an amino acid sequence at least 95% identical in sequence to SEQ ID NO: 2, 4, 6, 8, 10, 12, 14, 16, 18, 20, 22, 24, 26, or 28.

Also provided for herein is the use of engineered NK cells as disclosed herein for the preparation of a medicament for the treatment of cancer and/or for the treatment of cancer.

Also provided are methods of treating cancer in a subject in need thereof, comprising administering to the subject a therapeutically effective amount of engineered NK as disclosed herein.

### BRIEF DESCRIPTION OF THE DRAWINGS

The descriptions of the figures below are related to experiments and results that represent non-limiting embodiments of the inventions disclosed herein.

FIGS. 1A and 1B depict a non-limiting examples of expansion protocol used to enhance the expansion of NK cells according to embodiments disclosed herein.

FIG. 2 depicts data comparing fold expansion of NK cells using various expansion methodologies, including non-limiting embodiments of those disclosed herein.

FIGS. 3A-3B depict data related to the expansion of NK cells under various conditions from four different donors. FIG. 3A shows flow cytometry data measuring expression of NKG2D on the surface of NK cells when expanded with feeder cells alone (top row) or using cytokine supplementation (bottom row). FIG. 3B measures the mean fluorescence intensity of (representing transduction with an NKG2D bearing chimeric receptor construct (NKX101) under the various conditions.

FIG. 4 shows data by related to NK cell cytotoxicity at various time points after expansion under conditions using feeder cells alone, or with cytokine supplementation.

FIGS. 5A-5B depict data related to expression of certain markers indicative of a memory phenotype by NK cells.

FIG. 6 shows in vivo data related to the anti-tumor activity of NK cells expanded with or without the indicated cytokine stimulation during expansion.

FIGS. 7A-7B relate to NK cell expansion under various conditions. FIG. 7A shows the various concentrations determined to be over-saturated, saturated, or sub-saturated for IL12/18. FIG. 7B shows NK cell proliferation data under various culture conditions.

FIG. 8 shows data related to the release of interferon gamma by NK cells cultured in with varying concentrations of IL12 and/or IL18 in the culture media.

FIGS. 9A-9H relate to assessment of NK cell expansion after seven days of culture in the indicated conditions. FIG. 9A shows summary data for each of the culture groups. FIG. 9B provides statistical comparisons of the groups. FIG. 9C shows fold expansion data (at Day7) for a specific titration data set involving various concentrations of IL12 with IL18 at 4 ng/ml. FIG. 9D shows similar data with IL18 at 20 ng/ml. FIG. 9E shows viability of engineered NK cells at day 7 of culture with 20 ng/mL IL18, 40 IU/mL IL2 and the indicated concentrations of IL12. FIG. 9F shows viability of engineered NK cells at day 8 of culture with 20 ng/mL IL18, 400 IU/mL IL2 and the indicated concentrations of IL12. FIG. 9G shows viability of engineered NK cells at day 7 of culture with 4 ng/mL IL18, 40 IU/mL IL2 and the indicated concentrations of IL12. FIG. 9H shows viability of engineered NK cells at day 8 of culture with 4 ng/mL IL18, 400 IU/mL IL2 and the indicated concentrations of IL12.

FIGS. 10A-10B related to assessment of NK cell cytotoxicity. FIG. 10A shows summary data for the cytotoxicity of NK cells in each of the culture groups after 8 days of culture. FIG. 10B provides statistical comparisons of the cytotoxicity.

FIGS. 11A-11B related to assessment of NK cell cytotoxicity. FIG. 11A shows summary data for the cytotoxicity of NK cells in each of the culture groups after 15 days of culture. FIG. 11B provides statistical comparisons of the cytotoxicity.

FIG. 12 shows expression data for NK cells transduced with a chimeric receptor construct and cultured in various conditions from two donors.

FIG. 13 shows expression data for NK cells transduced with a chimeric receptor construct and cultured in various conditions from two additional donors.

FIGS. 14A-14B show cytotoxicity data. FIG. 14A shows summary data related to the cytotoxicity of NK cells transduced with a chimeric receptor targeting NKG2D ligands and cultured in the indicated conditions. FIG. 14B shows statistical comparisons of the groups.

FIGS. 15A-15D relate to cytotoxic effects of NK cells transduced with an NKG2D targeting chimeric receptor after being cultured under the indicated conditions. FIGS. 15A and 15B show data regarding cytotoxicity of NK cells from two different donors 13 days-post transduction with either a GFP-encoding vector or a vector encoding a chimeric receptor targeting NKG2D ligands. FIGS. 15C and 15D show corresponding cytotoxicity data from the same two donors at day 21 post-transduction.

FIGS. 16A-16B show data related to the phenotype of NK cells. FIG. 16A shows data related to the expression of markers associated with a memory-like phenotype by NK cells over time in the indicated culture conditions. FIG. 16B shows flow cytometry data showing the progression of marker expression over time in culture.

FIGS. 17A-17D shows summary expression data related to selected markers by NK cells in various culture conditions. FIG. 17A shows expression data related to CD62 ligand, FIG. 17B shows expression of NKG2C, FIG. 17C shows expression of CD57, and FIG. 17D shows expression of both CD62L and NKG2C.

FIG. 18 shows cytotoxicity data for NK cells expressing either GFP and or an NKG2D-ligand directed chimeric receptor at day 21 post-transduction.

FIG. 19 shows cell viability and expansion data for NK cells grown under varied culture conditions.

FIG. 20 shows expression data (based on a Flag tag) for NK cells transduced with an anti-CD19 CAR and cultured using the indicated conditions. This data was collected at day 15 of expansion.

FIG. 21 shows expression data (based on a Flag tag) for NK cells transduced with an anti-CD19 CAR and cultured using the indicated conditions. This data was collected at day 22 of expansion.

FIGS. 22A-22C show data related to the cytotoxicity of NK cells expressing an anti-CD19 CAR. NK cells were expanded using the indicated conditions and challenged with Nalm6 cells using the indicated E:T ratios in FIG. 22A (mean of 3 donors). FIG. 22B shows summary cytotoxicity data. FIG. 22C shows cytotoxicity data as a function of effector to target ratio.

FIG. 23 shows a schematic of an experimental setup to assess the cytotoxicity of NK cells expressing a chimeric receptor targeting NKG2D ligands in a hepatocellular carcinoma xenograft model.

FIG. 24 shows a summary of tumor burden over time in mice under the indicated treatments.

FIG. 25 shows a schematic experimental setup to assess the impact of expansion culture conditions on the cytotoxicity of NK cells in vivo.

FIGS. 26A-26F show cytotoxicity, survival data, data related to NK cell persistence, and data related to CAR expression in fresh or cryopreserved NK cells. FIG. 26A shows data related to the cytotoxicity of NK cells expanded under the indicated conditions against Nalm6 cells in a xenograft model. FIG. 26B shows a survival curve for mice receiving the indicated treatments. FIG. 26C shows data related to the detection of human NK cells in the murine blood 18 days post-injection, separated based on the expansion culture conditions. FIG. 26D shows data related to the detection of CAR-positive NK cells in the murine blood 18 days post-injection, separated based on the expansion culture conditions. FIG. 26E shows expression data related to the percentage of NK cells (either fresh or cryopreserved) expressing a non-limiting embodiment of an anti-CD19 CAR at day 15 of expansion and in the presence or absence of additional stimulatory molecules. FIG. 26F shows expression data related to the percentage of NK cells (either fresh or cryopreserved) expressing a non-limiting embodiment of an anti-CD19 CAR at day 22 of expansion and in the presence or absence of additional stimulatory molecules.

FIGS. 27A-27C relate to the in vivo efficacy of various CD19-directed CAR according to embodiments disclosed herein. FIG. 27A shows a schematic depiction of an experimental protocol for assessing the effectiveness of humanized, NK cells expressing various CD19-directed CAR constructs in vivo. The various experimental groups tested are as indicated. For cells with an “IL12/IL18” designation, the cells were expanded in the presence of soluble IL12 and/or IL18, according to embodiments disclosed herein. FIGS. 27B and 27C show bioluminescence data from animals dosed with Nalm6 tumor cells and treated with the indicated construct.

FIGS. 28A-28J show graphical depictions of the bioluminescence data from FIGS. 27B-27C. FIG. 28A shows bioluminescence (as photon/second flux) from animals receiving un-transduced NK cells. FIG. 28B shows flux measured in animals receiving PBS as a vehicle. FIG. 28C shows flux measured in animals receiving previously frozen NK cells expressing the NK19 NF2 CAR (as a non-limiting example of a CAR). FIG. 28D shows flux measured in animals receiving previously frozen NK cells expressing the NK19 NF2 CAR (as a non-limiting example of a CAR) expanded using IL12 and/or IL18. FIG. 28E and FIG. 28F show flux measured in animals receiving fresh NK cells expressing the NK19 NF2 CAR (as a non-limiting example of a CAR). FIG. 28G and FIG. 28H show flux measured in animals receiving previously fresh NK cells expressing the NK19 NF2 CAR (as a non-limiting example of a CAR) expanded using IL12 and/or IL18. FIG. 28I shows a line graph depicting the bioluminescence measured in the various groups over the first 30 days post-tumor inoculation. FIG. 28J shows a line graph depicting the bioluminescence measured in the various groups over the first 56 days post-tumor inoculation.

FIG. 29 shows data related to the body mass of mice over time when receiving the indicated therapy.

FIGS. 30A-30C show data related to data characterizing NK cells engineered to express CARs (as disclosed herein) and expanded in the presence or absence of one or more stimulatory cytokines. FIG. 30A shows data related to the percentage of NK cells expressing CARs in the blood of animals over time. FIG. 30B shows data related to the percentage of NK cells expressing CARs in the blood of animals over a period of 50 days. FIG. 30C shows data related to the percentage of NK cells expressing CARs over time and based on the number of live cells tested.

FIGS. 31A-31C show data from three different mice (31A, 31B, and 31C, respectively) related the expression of an anti-CD19 CAR and characterization of what cells express the CAR.

FIGS. 32A-32C show data from three different mice (32A, 32B, and 32C, respectively) related the expression of an anti-CD19 CAR and characterization of what cells express the CAR.

FIGS. 33A-33C show summary expression data from blood samples collected 4 days after in vivo administration (protocol of FIG. 27A). FIG. 33A shows the percentage of CD3⁻CD56⁺ NK cells from in whole blood samples for the indicated experimental groups. FIG. 33B shows the percentage of NK cells expressing a specific anti-CD19 CAR for each experimental group. FIG. 33C shows data relating to the number of GFP positive tumor cells detected for each experimental group.

FIGS. 34A-34C show summary expression data from blood samples collected 12 days after in vivo administration (protocol of FIG. 27A). FIG. 34A shows the percentage of CD3⁻CD56⁺ NK cells from in whole blood samples for the indicated experimental groups. FIG. 34B shows the percentage of NK cells expressing a specific anti-CD19 CAR for each experimental group. FIG. 34C shows data relating to the number of GFP positive tumor cells detected for each experimental group.

FIGS. 35A-35E show summary expression data from blood samples collected 18 days after in vivo administration (protocol of FIG. 27A). FIG. 35A shows the percentage of CD3⁻CD56⁺ NK cells from whole blood samples for the indicated experimental groups. FIG. 35B shows the percentage of CD19-positive tumor cells for each experimental group as measured using a phycoerythrin (PE)-conjugated antibody. FIG. 35C shows data relating to the number of GFP positive tumor cells detected for each experimental group. FIG. 35D shows the percentage of NK cells expressing a specific anti-CD19 CAR for each experimental group as measured using an anti CD19 FC antibody. FIG. 35E shows the percentage of NK cells in each treatment group expressing the CD19 CAR.

FIG. 36 shows data collected over 4 weeks relating to the half-life of NK cells expressing an anti-CD19 CAR, for each of two doses of NK cells, as measured by the count of NK cells per 10,000 leukocytes. The two doses were (i) 2 million NK cells expressing an anti-CD19 CAR and (ii) 5 million NK cells expressing an anti-CD19 CAR. These data were collected after a third dose of NK cells were administered.

FIG. 37 shows data collected for the half-life of cryopreserved NK cells engineered to express a CAR targeting NKG2D ligands and expanded without the use of an additional stimulatory cytokine.

FIGS. 38A-38D shows comparative dose-response cytotoxicity data of various CD19-targeting CARs (or non-transduced NK cells) against tumor cell lines. FIG. 38A shows cytotoxicity against high CD19-expressing Nalm6 tumor cells after 24 hours. FIG. 38B shows the cytotoxicity against Nalm6 after 72 hours. FIG. 38C shows cytotoxicity, after 24 hours, against Reh tumor cells which exhibit lower levels of CD19 than Nalm6 cells. FIG. 38D shows cytotoxicity against Reh cells after 72 hours.

FIGS. 39A-39B show data relating to the cytotoxicity of NK cells (39A) or NK cells (39A) expressing a non-limiting embodiment of a CD19-CAR and the in the presence and absence of dexamethasone.

FIGS. 40A-40B show data (different scales between 40A and 40B) related to the lifespan (e.g., half-life) of CAR-expressing NK (data shown is a non-limiting embodiment of a CAR, here a CD19-targeting CAR) cells in the blood stream of animals over time.

FIGS. 41A-41B relate to NK cell expansion data over time where NK cells, obtained from either cord blood (CB) or peripheral blood (PB) were pulsed with feeder cells at a 1:10 ratio at multiple time points during the expansion process, in conjunction with supplementing the media with IL2. FIG. 41A shows a line graph of the expansion of the NK cells over time. FIG. 41B shows summary expansion data.

FIGS. 42A-42B relate to NK cell expansion data over time where NK cells, obtained from either cord blood (CB) or peripheral blood (PB) were pulsed with feeder cells at a 1:10 ratio at multiple time points during the expansion process, in conjunction with supplementing the media with both IL2 and IL12. FIG. 42A shows a line graph of the expansion of the NK cells over time. FIG. 42B shows summary expansion data.

FIGS. 43A-43B relate to NK cell expansion data over time where NK cells, obtained from either cord blood (CB) or peripheral blood (PB) were pulsed with feeder cells at a 1:10 ratio at multiple time points during the expansion process, in conjunction with supplementing the media with both IL2 and IL18. FIG. 43A shows a line graph of the expansion of the NK cells over time. FIG. 43B shows summary expansion data.

FIGS. 44A-44B relate to NK cell expansion data over time where NK cells, obtained from either cord blood (CB) or peripheral blood (PB) were pulsed with feeder cells at a 1:10 ratio at multiple time points during the expansion process, in conjunction with supplementing the media with both IL2 and a combination of IL12 and IL18. FIG. 44A shows a line graph of the expansion of the NK cells over time. FIG. 44B shows summary expansion data.

FIGS. 45A-45E relate to the expansion of CD3-positive cells as a result of multiple pulses of feeder cells and the indicated cytokine conditions used to supplement the media. Stars in the Figures indicate when NK cells were replated on fresh feeder cells with fresh media (including supplementing media components, if indicated). FIG. 45A shows data when high concentrations of IL2 (400 units/mL) were used to supplement the media. FIG. 45B shows data related to supplementing the media with IL12 (as well as 40 units/mL IL2). FIG. 45C shows data related to supplementing the media with IL18 (as well as 40 units/mL IL2). FIG. 45D shows data related to supplementing the media with both IL12 and IL18 (as well as 40 units/mL IL2). FIG. 45E shows data related to control expansion (pulsing with new feeder cells and 40 units/mL IL2, but no additional cytokines).

FIGS. 46A-46J related to expression of the activating NKG2C receptor. Stars in the Figures indicate when NK cells were replated on fresh feeder cells with fresh media (including supplementing media components, if indicated). FIG. 46A shows data when high concentrations of IL2 (400 units/mL) were used to supplement the media with data presented as the percentage of NK cells that are positive for NKG2C expression. FIG. 46B shows data when high concentrations of IL2 (400 units/mL) were used to supplement the media with data presented as the overall Mean Fluorescence Intensity (MFI) to represent NKG2C expression. FIG. 46C shows data related to supplementing the media with IL12 (as well as 40 units/mL IL2) with data presented as the percentage of NK cells that are positive for NKG2C expression. FIG. 46D shows data related to supplementing the media with IL12 (as well as 40 units/mL IL2) with data presented as the overall MFI to represent NKG2C expression. FIG. 46E shows data related to supplementing the media with IL18 (as well as 40 units/mL IL2) with data presented as the percentage of NK cells that are positive for NKG2C expression. FIG. 46F shows data related to supplementing the media with IL18 (as well as 40 units/mL IL2) with data presented as the overall MFI to represent NKG2C expression. FIG. 46G shows data related to supplementing the media with both IL12 and IL18 (as well as 40 units/mL IL2) with data presented as the percentage of NK cells that are positive for NKG2C expression. FIG. 46H shows data related to supplementing the media with both IL12 and IL18 (as well as 40 units/mL IL2) with data presented as the overall MFI to represent NKG2C expression. FIG. 45I shows data related to control expansion (pulsing with new feeder cells and 40 units/mL IL2, but no additional cytokines) with data presented as the percentage of NK cells that are positive for NKG2C expression. FIG. 45J shows data related to control expansion (pulsing with new feeder cells and 40 units/mL IL2, but no additional cytokines) with data presented as the overall MFI to represent NKG2C expression.

FIGS. 47A-47B show an increase in activation (47A) and inhibitory (47B) markers on NK cells expanded with two feeder pulses (“2X”) on Day 0 and Day 7 or three feeder pulses (“3X”) on Day 0, Day 7 and Day 14, as compared to an expansion method with a single feeder stimulation at Day 0 (“SOP”). “Pure NK” are groups in which the initial population of cells cultured with the feeder cells were purified. “PBM” are groups in which the initial population of cells cultured with the feeder cells were peripheral blood mononuclear cells, not purified NK cells.

FIGS. 48A-48B relate to additional evaluation of the expression of activation (48A) or inhibitory (48B) markers at Day 0 (circles) or Day 7 (squares).

### DETAILED DESCRIPTION

While cancer immunotherapy, or cellular therapy for other diseases, has advanced greatly in terms of the ability to engineer cells to express constructs of interest, there is still a need for clinically relevant number of those cells for patient administration. This is particularly important when the underlying native immune cell to be engineered and later administered is less prevalent than other immune cell types. This requires either starting with a larger amount of starting material, which may not be practical, or developing more efficient methods and compositions to expand (in some cases preferentially) the immune cell of interest, such as an NK cell. There are therefore provided herein, in several embodiments, methods and compositions that advantageously allow for the enhanced expansion of NK cells (or other immune cells) but also allow for enhanced cytotoxicity of those cells.

In several embodiments, there are provided populations of expanded and activated NK cells derived from co-culturing a modified “feeder” cell disclosed herein with a starting population of immune cells and supplementing the co-culture with various cytokines at certain time points during the expansion.

### Cells for Use in Immune Cell Expansion

In several embodiments, cell lines are used in a co-culture with a population of immune cells that are to be expanded. Such cell lines are referred to herein as “stimulatory cells,” which can also be referred to as “feeder cells”. In several embodiments, the entire population of immune cells is to be expanded, while in several embodiments, a selected immune cell subpopulation is to be expanded. For example, in several embodiments, NK cells are expanded relative to other immune cell subpopulations (such as T cells). In other embodiments, both NK cells and T cells are expanded. In several embodiments, the feeder cells are themselves genetically modified. In some embodiments, the feeder cells do not express MHC I molecules, which have an inhibitory effect on NK cells. In some embodiments, the feeder cells need not entirely lack MHC I expression, however they may express MHC I molecules at a lower level than a wild type cell. For example, in several embodiments, if a wild type cell expresses an MHC at a level of X, the cell lines used may express MHC at a level less than 95% of X, less than 90% of X, less than 85% of X, less than 80% of X, less than 70% of X, less than 50% of X, less than 25% of X, and any expression level between (and including) those listed. In several embodiments, the stimulatory cells are immortalized, e.g., a cancer cell line. However, in several embodiments, the stimulatory cells are primary cells.

Various cell types can be used as feeder cells, depending on the embodiment. These include, but are not limited to, K562 cells, certain Wilms Tumor cell lines (for example Wilms tumor cell line HFWT), endometrial tumor cells (for example, HHUA), melanoma cells (e.g., HMV-II), hepatoblastoma cells (e.g., HuH-6), lung small cell carcinoma cells (e.g., Lu-130 and Lu-134-A), neuroblastoma cells (e.g., NB19 and NB69), embryonal carcinoma testis cells (e.g., NEC14), cervical carcinoma cells (TCO-2), neuroblastoma cells (e.g., TNB1), 721.221 EBV transformed B cell line, among others.

In additional embodiments, the feeder cells also have reduced (or lack) MHC II expression, as well as having reduced (or lacking) MHC I expression. In some embodiments, other cell lines that may initially express MHC class I molecules can be used, in conjunction with genetic modification of those cells to reduce or knock out MHC I expression. Genetic modification can be accomplished through the use of gene editing techniques (e.g. a CRISPR/Cas system; RNA editing with an Adenosine deaminases acting on RNA (ADAR), zinc fingers, TALENS, etc.), inhibitory RNA (e.g., siRNA), or other molecular methods to disrupt and/or reduce the expression of MHC I molecules on the surface of the cells.

As discussed in more detail below, in several embodiments, the feeder cells are engineered to express certain stimulatory molecules (e.g. interleukins, CD3, 4-1 BBL, etc.) to promote immune cell expansion and activation. Engineered feeder cells are disclosed in, for example, International Patent Application PCT/SG2018/050138, which is incorporated in its entirety by reference herein. In several embodiments, the stimulatory molecules, such as interleukin 12, 18, and/or 21 are separately added to the co-culture media, for example at defined times and in particular amounts, to effect an enhanced expansion of a desired sub-population(s) of immune cells.

### Stimulatory Molecules

As discussed briefly above, certain molecules promote the expansion of immune cells, such as NK cells or T cells, including engineered NK or T cells. Depending on the embodiment, the stimulatory molecule, or molecules, can be expressed on the surface of the feeder cells used to expand the immune population. For example, in several embodiments a K562 feeder cell population is engineered to express 4-1BBL and/or membrane bound interleukin 15 (mbIL15). Additional embodiments relate to further membrane bound interleukins or stimulatory agents. Examples of such additional membrane bound stimulatory molecules can be found in International Patent Application PCT/SG2018/050138, which is incorporated in its entirety by reference herein.

In several embodiments, the methods disclosed herein relate to addition of one or more stimulatory molecules to the culture media in which engineered feeder cells and engineered NK cells are co-cultured. In several embodiments, one or more interleukins is added. For example, in several embodiments, IL2 is added to the media. In several embodiments, IL12 is added to the media. In several embodiments, IL18 is added to the media. In several embodiments, IL21 is added to the media. In several embodiments, combinations of two or more of IL2, IL12, IL18, and/or IL21 is added to the media. In some embodiments, rather than using a feeder cell with mbIL15, soluble IL15 is added to the media (alone or in combination with any of IL2, IL12, IL18, and IL21).

In several embodiments, the media comprises one or more vitamin, inorganic salt and/or amino acids. In several embodiments, the media comprises 1, 2, 3, 4, 5, 6, 7, 8, 9, 10 or all of Glycine, L-Arginine, L-Asparagine, L-Aspartic acid, L-Cystine (e.g., L-Cystine 2HCl), L-Glutamic Acid, L-Glutamine, L-Histidine, L-Hydroxyproline, L-Isoleucine, L-Leucine, L-Lysine hydrochloride, L-Methionine, L-Phenylalanine, L-Proline, L-Serine, L-Threonine L-Tryptophan, L-Tyrosine (e.g., L-Tyrosine disodium salt dehydrate), and L-Valine. In several embodiments, the media comprises 1, 2, 3, 4, or more of Biotin, Choline chloride, D-Calcium pantothenate, Folic Acid, i-Inositol, Niacinamide, Para-Aminobenzoic Acid, Pyridoxine hydrochloride, Riboflavin, Thiamine hydrochloride, and Vitamin B12. In several embodiments, the media comprises 1, 2, 3, 4, or more of Calcium nitrate (Ca(NO₃)2 4H₂O), Magnesium Sulfate (MgSO₄) (e.g., Magnesium Sulfate (MgSO₄) (anhyd.)), Potassium Chloride (KCl), Sodium Bicarbonate (NaHCO₃), Sodium Chloride (NaCl), and Sodium Phosphate dibasic (Na₂HPO₄) (e.g., Sodium Phosphate dibasic (Na₂HPO₄) anhydrous).

In several embodiments, the media further comprises D-Glucose and/or glutathione (optionally reduced glutathione). In several embodiments, the media further comprises serum (e.g., fetal bovine serum) in an amount ranging from about 1% to about 20%. In several embodiments, the serum is heat-inactivated. In several embodiments, the media is serum-free. In several embodiments, the media is xenofree.

Depending on the embodiment, IL2 is used to supplement the culture media and enhance expansion, or other characteristics, of NK cells. In several embodiments, the concentration of IL2 used ranges from about 1 IU/mL to about 1000 IU/mL, including for example, about 1 IU/mL to about 5 IU/mL (e.g., 1, 2, 3, 4, and 5), about 5 IU/mL to about 10 IU/mL (e.g., 5, 6, 7, 8, 9, and 10), about 10 IU/mL to about 20 IU/mL (e.g., about 10, 12, 14, 16, 18, and 20), about 20 IU/mL to about 30 IU/mL (e.g., about 20, 22, 24, 26, 28, and 30), about 30 IU/mL to about 40 IU/mL (e.g., 30, 32, 34, 36, 38, and 40), about 40 to about 50 IU/mL (e.g., 40, 42, 44, 46, 48, 50), about 50 IU/mL to about 75 IU/mL (e.g., 50, 55, 60, 65, 70, and 75), about 75 IU/mL to about 100 IU/mL (e.g., 75, 80, 85, 90, 95, and 100), about 100 IU/mL to about 200 IU/mL (e.g., 100, 125, 150, 275, and 200), about 200 IU/mL to about 300 IU/mL (e.g., 200, 225, 250, 275, and 300), about 300 IU/mL to about 400 IU/mL (e.g., 300, 325, 350, 375, and 400), about 400 IU/mL to about 500 IU/mL (e.g., 400, 425, 450, 475, and 500), about 500 IU/mL to about 750 IU/mL (e.g., 500, 550, 600, 650, 700, and 750), or about 750 IU/mL to about 1000 IU/mL (e.g., 750, 800, 850, 900, 950, and 1000), and any concentration therebetween, including endpoints. In several embodiments, IL2 may be added at multiple time points during culture. In some such embodiments, the concentration of IL2 used may differ between selected time points.

As used herein, and conventionally understood in the art, the terms “units” and “international unit (IU)” refers to a standardized amount or measure of a substance, molecule, or compound as determined by a measurement of activity, such as a biological activity. For the purposes of this disclosure, the terms “units” and “IU” are interchangeable. As generally understood, a measurement by units or IU may or may not be advantageous compared to other conventional modes of quantification such as mass or volume, as it enables correlation of the same substance between, e.g. different manufacturing processes or batches. As applied to IL2 as used herein, the definition of a unit or IU of IL2 is standardized according to the WHO International Standard for IL2 under NIBSC code 86/500. It will be understood by a skilled person that the disclosure of IL2 concentrations by measurements of units/mL or IU/mL should be transferrable, without undue experimentation, regardless of the source of IL2 used as long as it is manufactured according to the NIBSC standard.

For the purposes of the disclosure herein, the IL2 used has a concentration of 40 IU/mL corresponding to 2.3 ng/mL (i.e. 1 IU/mL corresponds to 57.5 pg/mL). Accordingly, any concentration of IL2 in terms of IU/mL or units/mL may be interpreted in terms of their mass concentration according to this equivalence. It will be understood that one skilled in the art will be able to determine their corresponding mass concentration equivalent of an IL2 product measured in terms of IU/mL if alternatives are used.

Depending on the embodiment, IL2 is used to supplement the culture media and enhance expansion, or other characteristics, of NK cells. In several embodiments, the concentration of IL2 used ranges from 57.5 pg/mL to about 57.5 ng/mL (or from 55 pg/mL to about 60 ng/mL), including for example, about 55 pg/mL to about 500 pg/mL (e.g., 55, 60, 100, 200, 300, 400, 500 pg/mL), about 500 pg/mL to about 1000 pg/mL (e.g. 500, 600, 700, 800, 900, 1000 pg/mL), about 1 ng/mL to about 10 ng/mL (e.g., 1, 2, 3, 4, 5, 6, 7, 8, 9, 10 ng/mL), or about 10 ng/mL to about 60 ng/mL (e.g., 10, 15, 20, 25, 30, 35, 40, 45, 50, 55, 60 ng/mL, and any concentration therebetween, including endpoints. In several embodiments, IL2 may be added at multiple time points during culture. In some such embodiments, the concentration of IL2 used may differ between selected time points.

Depending on the embodiment, IL12A and/or IL12B is used to supplement the culture media and enhance expansion, or other characteristics, of NK cells. In several embodiments, the concentration of IL12 (either IL12A or IL12B) used ranges from about 0.01 ng/ml to about 100 ng/mL, including, for example, about 0.01 ng/mL to about 0.05 ng/mL (e.g., 0.01, 0.02, 0.03, 0.04, and 0.05), about 0.05 ng/mL to about 0.1 ng/mL (e.g., 0.05, 0.06, 0.07, 0.08, 0.09 and 0.1), about 0.1 ng/mL to about 0.5 ng/mL (e.g., 0.1, 0.2, 0.3, 0.4, and 0.5), about 0.5 ng/mL to about 1.0 ng/mL (e.g., 0.5, 0.6, 0.7, 0.8, 0.9, and 1.0), about 1.0 ng/mL to about 2.0 ng/mL (e.g., 1.1, 1.2, 1.3, 1.4, 1.5, 1.6, 1.7, 1.8, 1.9, and 2.0), about 2.0 ng/mL to about 5.0 ng/mL (e.g., 2.0, 3.0, 4.0, and 5.0), about 5.0 ng/mL to about 10.0 ng/mL (e.g., 5.0, 6.0, 7.0, 8.0, 9.0 and 10.0), about 10.0 ng/mL to about 15.0 ng/mL (e.g., 10.0, 11.0, 12.0, 13.0, 14.0, and 15.0), about 15.0 ng/mL to about 20.0 ng/mL (e.g., 15.0, 16.0, 17.0, 18.0, 19.0, and 20.0), about 20.0 ng/mL to about 25.0 ng/mL (e.g., 20.0, 21.0, 22.0, 23.0, 24.0, and 25.0), about 25.0 ng/mL to about 30.0 ng/mL (e.g., 25.0, 26.0, 27.0, 28.0, 29.0, and 30.0), about 30.0 ng/mL to about 50.0 ng/mL (e.g., 30.0, 35.0, 40.0, 45.0, and 50.0), about 50.0 ng/mL to about 75.0 ng/mL (e.g., 50.0, 55.0, 60.0, 65.0, 70.0, and 75.0), about 75.0 ng/mL to about 100.0 ng/mL (e.g., 75.0, 80.0, 85.0, 90.0, 95.0, and 100.0), and any concentration therebetween, including endpoints. In several embodiments, the concentration of IL12 is between about 0.01 ng/mL and about 8 ng/mL, including any concentration therebetween, including endpoints.

In some embodiments, a mixture of IL12A and IL12B is used. In several embodiments, a particular ratio of IL12A:IL12B is used, for example, 1:10, 1:50, 1:100, 1:150, 1:200, 1:250:, 1:500, 1:1000, 1:10,000, 10,000:1, 1000:1, 500:1, 250:1, 150:1, 100:1, 10:1 and any ratio there between, including endpoint.

In some embodiments, interleukin 18 (IL18) is used to enhance expansion, or other characteristics, of NK cells. In several embodiments, the concentration of IL18 used ranges from about 0.01 ng/ml to about 100 ng/mL, including, for example, about 0.01 ng/mL to about 0.05 ng/mL (e.g., 0.01, 0.02, 0.03, 0.04, and 0.05), about 0.05 ng/mL to about 0.1 ng/mL (e.g., 0.05, 0.06, 0.07, 0.08, 0.09 and 0.1), about 0.1 ng/mL to about 0.5 ng/mL(e.g., 0.1, 0.2, 0.3, 0.4, and 0.5), about 0.5 ng/mL to about 1.0 ng/mL (e.g., 0.5, 0.6, 0.7, 0.8, 0.9, and 1.0), about 1.0 ng/mL to about 2.0 ng/mL (e.g., 1.1, 1.2, 1.3, 1.4, 1.5, 1.6, 1.7, 1.8, 1.9, and 2.0), about 2.0 ng/mL to about 5.0 ng/mL (e.g., 2.0, 3.0, 4.0, and 5.0), about 5.0 ng/mL to about 10.0 ng/mL (e.g., 5.0, 6.0, 7.0, 8.0, 9.0 and 10.0), about 10.0 ng/mL to about 15.0 ng/mL (e.g., 10.0, 11.0, 12.0, 13.0, 14.0, and 15.0), about 15.0 ng/mL to about 20.0 ng/mL (e.g., 15.0, 16.0, 17.0, 18.0, 19.0, and 20.0), about 20.0 ng/mL to about 25.0 ng/mL (e.g., 20.0, 21.0, 22.0, 23.0, 24.0, and 25.0), about 25.0 ng/mL to about 30.0 ng/mL (e.g., 25.0, 26.0, 27.0, 28.0, 29.0, and 30.0), about 30.0 ng/mL to about 50.0 ng/mL (e.g., 30.0, 35.0, 40.0, 45.0, and 50.0), about 50.0 ng/mL to about 75.0 ng/mL (e.g., 50.0, 55.0, 60.0, 65.0, 70.0, and 75.0), about 75.0 ng/mL to about 100.0 ng/mL (e.g., 75.0, 80.0, 85.0, 90.0, 95.0, and 100.0), and any concentration therebetween, including endpoints.

In some embodiments interleukin 21 (IL21) is used to enhance expansion, or other characteristics, of NK cells. In several embodiments, the concentration of IL21 used ranges from about 0.01 ng/ml to about 100 ng/mL, including, for example, about 0.01 ng/mL to about 0.05 ng/mL (e.g., 0.01, 0.02, 0.03, 0.04, and 0.05), about 0.05 ng/mL to about 0.1 ng/mL (e.g., 0.05, 0.06, 0.07, 0.08, 0.09 and 0.1), about 0.1 ng/mL to about 0.5 ng/mL(e.g., 0.1, 0.2, 0.3, 0.4, and 0.5), about 0.5 ng/mL to about 1.0 ng/mL (e.g., 0.5, 0.6, 0.7, 0.8, 0.9, and 1.0), about 1.0 ng/mL to about 2.0 ng/mL (e.g., 1.1, 1.2, 1.3, 1.4, 1.5, 1.6, 1.7, 1.8, 1.9, and 2.0), about 2.0 ng/mL to about 5.0 ng/mL (e.g., 2.0, 3.0, 4.0, and 5.0), about 5.0 ng/mL to about 10.0 ng/mL (e.g., 5.0, 6.0, 7.0, 8.0, 9.0 and 10.0), about 10.0 ng/mL to about 15.0 ng/mL (e.g., 10.0, 11.0, 12.0, 13.0, 14.0, and 15.0), about 15.0 ng/mL to about 20.0 ng/mL (e.g., 15.0, 16.0, 17.0, 18.0, 19.0, and 20.0), about 20.0 ng/mL to about 25.0 ng/mL (e.g., 20.0, 21.0, 22.0, 23.0, 24.0, and 25.0), about 25.0 ng/mL to about 30.0 ng/mL (e.g., 25.0, 26.0, 27.0, 28.0, 29.0, and 30.0), about 30.0 ng/mL to about 50.0 ng/mL (e.g., 30.0, 35.0, 40.0, 45.0, and 50.0), about 50.0 ng/mL to about 75.0 ng/mL (e.g., 50.0, 55.0, 60.0, 65.0, 70.0, and 75.0), about 75.0 ng/mL to about 100.0 ng/mL (e.g., 75.0, 80.0, 85.0, 90.0, 95.0, and 100.0), and any concentration therebetween, including endpoints.

In some embodiments interleukin 15 (IL15) is used in a soluble format (either in place of, or in addition to mbIL15 on the feeder cells) to enhance expansion, or other characteristics, of NK cells. In several embodiments, the concentration of IL15 used ranges from about 0.01 ng/ml to about 100 ng/mL, including, for example, about 0.01 ng/mL to about 0.05 ng/mL (e.g., 0.01, 0.02, 0.03, 0.04, and 0.05), about 0.05 ng/mL to about 0.1 ng/mL (e.g., 0.05, 0.06, 0.07, 0.08, 0.09 and 0.1), about 0.1 ng/mL to about 0.5 ng/mL (e.g., 0.1, 0.2, 0.3, 0.4, and 0.5), about 0.5 ng/mL to about 1.0 ng/mL (e.g., 0.5, 0.6, 0.7, 0.8, 0.9, and 1.0), about 1.0 ng/mL to about 2.0 ng/mL (e.g., 1.1, 1.2, 1.3, 1.4, 1.5, 1.6, 1.7, 1.8, 1.9, and 2.0), about 2.0 ng/mL to about 5.0 ng/mL (e.g., 2.0, 3.0, 4.0, and 5.0), about 5.0 ng/mL to about 10.0 ng/mL (e.g., 5.0, 6.0, 7.0, 8.0, 9.0 and 10.0), about 10.0 ng/mL to about 15.0 ng/mL (e.g., 10.0, 11.0, 12.0, 13.0, 14.0, and 15.0), about 15.0 ng/mL to about 20.0 ng/mL (e.g., 15.0, 16.0, 17.0, 18.0, 19.0, and 20.0), about 20.0 ng/mL to about 25.0 ng/mL (e.g., 20.0, 21.0, 22.0, 23.0, 24.0, and 25.0), about 25.0 ng/mL to about 30.0 ng/mL (e.g., 25.0, 26.0, 27.0, 28.0, 29.0, and 30.0), about 30.0 ng/mL to about 50.0 ng/mL (e.g., 30.0, 35.0, 40.0, 45.0, and 50.0), about 50.0 ng/mL to about 75.0 ng/mL (e.g., 50.0, 55.0, 60.0, 65.0, 70.0, and 75.0), about 75.0 ng/mL to about 100.0 ng/mL (e.g., 75.0, 80.0, 85.0, 90.0, 95.0, and 100.0), and any concentration therebetween, including endpoints.

In some embodiments interleukin 22 (IL22) is used to facilitate expansion of NK cells. In several embodiments, the concentration of IL22 used ranges from about 0.01 ng/ml to about 100 ng/mL, including, for example, about 0.01 ng/mL to about 0.05 ng/mL (e.g., 0.01, 0.02, 0.03, 0.04, and 0.05), about 0.05 ng/mL to about 0.1 ng/mL (e.g., 0.05, 0.06, 0.07, 0.08, 0.09 and 0.1), about 0.1 ng/mL to about 0.5 ng/mL(e.g., 0.1, 0.2, 0.3, 0.4, and 0.5), about 0.5 ng/mL to about 1.0 ng/mL (e.g., 0.5, 0.6, 0.7, 0.8, 0.9, and 1.0), about 1.0 ng/mL to about 2.0 ng/mL (e.g., 1.1, 1.2, 1.3, 1.4, 1.5, 1.6, 1.7, 1.8, 1.9, and 2.0), about 2.0 ng/mL to about 5.0 ng/mL (e.g., 2.0, 3.0, 4.0, and 5.0), about 5.0 ng/mL to about 10.0 ng/mL (e.g., 5.0, 6.0, 7.0, 8.0, 9.0 and 10.0), about 10.0 ng/mL to about 15.0 ng/mL (e.g., 10.0, 11.0, 12.0, 13.0, 14.0, and 15.0), about 15.0 ng/mL to about 20.0 ng/mL (e.g., 15.0, 16.0, 17.0, 18.0, 19.0, and 20.0), about 20.0 ng/mL to about 25.0 ng/mL (e.g., 20.0, 21.0, 22.0, 23.0, 24.0, and 25.0), about 25.0 ng/mL to about 30.0 ng/mL (e.g., 25.0, 26.0, 27.0, 28.0, 29.0, and 30.0), about 30.0 ng/mL to about 50.0 ng/mL (e.g., 30.0, 35.0, 40.0, 45.0, and 50.0), about 50.0 ng/mL to about 75.0 ng/mL (e.g., 50.0, 55.0, 60.0, 65.0, 70.0, and 75.0), about 75.0 ng/mL to about 100.0 ng/mL (e.g., 75.0, 80.0, 85.0, 90.0, 95.0, and 100.0), and any concentration therebetween, including endpoints.

If two stimulatory agents are used, the relative ratio between the two can range from a ratio of 1:10, 1:20, 1:50, 1:100, 1:150, 1:200, 1:250, 1:500, 1:750, 1:1,000, 1:10,000, 1:50,000, 1:100,000, 100,000:1, 50,000:1, 10,000:1, 1,000:1, 750:1, 500:1, 250:1, 200:1, 150:1, 100:1, 50:1, 20:1, 10:1, and any ratio in between those listed, including endpoints. Likewise, if three, or more, agents are used, the ratio between those additional agents and the other agents can employ any of the aforementioned ratios.

As discussed in more detail below, depending on the embodiment, the stimulatory molecules may be added at a specific point (or points) during the expansion process, or can be added such that they are present as a component of the culture medium through the co-culture process.

### Methods of Co-Culture and Immune Cell Expansion

In some embodiments, NK cells isolated from a peripheral blood donor sample are co-cultured with K562 cells modified to express 4-1 BBL and mbIL15. While other approaches involve the expression of other membrane-bound cytokines, the generation of a feeder cell with multiple stimulatory molecules can be difficult to generate (e.g., to achieve desired levels of expression of the various stimulatory molecule, expression at the right time during expansion, etc.). Thus, several embodiments disclosed herein relate to the supplementation of the culture media with particular concentrations of various stimulatory agents at particular times. In several embodiments, feeder cells are seeded into culture vessels and allowed to reach near confluence. Immune cells can then be added to the culture at a desired concentration, ranging, in several embodiments from about 0.5×10⁶ cells/cm² to about 5×10⁶ cells/cm², including any density between those listed, including endpoints.

In several embodiments, immune cells are separated from a peripheral blood sample. Thereafter, in several embodiments, the immune cells can be expanded together, or an isolated subpopulation of cells, such as NK cells, is used. In several embodiments, cord blood, or other sources of blood are used as a source of immune cells. Some embodiments employ a specific population, or subpopulation of immune cells. In several embodiments, the population or subpopulation is purified prior to expansion in culture. For example, in several embodiments, purified NK cells are used in expansion. In other embodiments, mononuclear cells (e.g., peripheral blood mononuclear cells or cord blood mononuclear cells) are the cells used in expansion.

Thereafter, the NK cells are seeded with the feeder cells, an optionally one or more cytokines (either in the culture media or as an exogenous supplement) and cultured for a first period of time, for example about 6 hours, about 12 hours, about 18 hours, about 24 hours, about 2 days, about 3 days, about 4 days, about 5 days, about 6 days, about 7 days, about 8 days, about 9 days, about 10 days, about 11 days, about 12 days, about 13 days, about 14 days, or for any time between those listed, including endpoints.

As discussed in additional detail in the Examples (see, e.g., Example 4) the cells being expanded are “pulsed” with fresh media and feeder cells, and optionally one or more of the stimulatory cytokines used to supplement the culture media. For example, in several embodiments, the cells being expanded are collected and replated in a culture vessel having a new “batch” of feeder cells therein. The cells being expanded are added to the new culture vessel/feeder cells with fresh media. In several embodiments, the media added to the co-culture is also fresh, including, optionally, supplementing the media with any of the stimulatory cytokines that were initially present in the expansion media (e.g., at Day 0 of expansion). As discussed herein, the concentration of the stimulatory cytokine(s) added to the media can be the same as at a prior expansion period, or optionally a different concentration (either higher or lower). In several embodiments, the cells being expanded are pulsed at least one additional time during expansion. In several embodiments, the cells being expanded are pulsed 2, 3, 4, 5, 6, 7, 8, 9, 10 or more times.

In several embodiments, the duration between a first and a second pulse is about 5 to 7 days. In several embodiments, the duration between a given first pulse and a given second pulse is about 2 days, about 3 days, about 4 days, about 5 days, about 6 days, about 7 days, about 8 days, about 9 days, about 10 days, about 11 days, about 12 days, about 13 days, or about 14 days (or any time between those listed, including endpoints). Depending on the embodiment, the duration between pulses is relatively constant, for example, if the time between Day 0 of expansion and the first pulse is about 5 to about 7 days, the duration from the first pulse and the second pulse is about 5 to about 7 days. However, in several embodiments, the time can be adjusted, for example for convenience or dur to an observed change in the health of the expanding cells. In several embodiments, the pulsed expansion allows for the cells to be expanded (such as NK cells) to continue to keep expanding to achieve at least 20,000-fold expansion from the initial cell count. In several embodiments, greater expansion is achieved, such as at least about 50,000-fold expansion, at least about 100,000-fold expansion, at least about 150,000-fold expansion, at least about 200,000-fold expansion, at least about 250,000-fold expansion, at least about 300,000-fold expansion, at least about 350,000-fold expansion, at least about 400,000-fold expansion, at least about 450,000-fold expansion, at least about 500,000-fold expansion, at least about 750,000-fold expansion, at least about 1,000,000-fold expansion, at least about 1,250,000-fold expansion, at least about 1,500,000-fold expansion, at least about 1,750,000-fold expansion, at least about 2,000,000-fold expansion, about 2,500,000-fold expansion, or about 3,000,000-fold expansion, or any degree of expansion between those listed, including endpoints. In several embodiments, greater than about 4,000,000 or about 5,000,000-fold expansion is achieved.

In several embodiments, the ratio of the number of immune cells to be expanded at the inception of expansion to the number of expanded cells at the end of expansion is about 1:25,000; about 1:50,000, about 1:100,000, about 1:200,000, about 1:500,000, about 1:1,000,000, about 1:1,500,000, about 1:2,000,000, about 1:2,500,000, or about 1:3,000,000, or any ratio between those listed, including endpoints.

The degree of expansion can be adjusted, in several embodiments, by adjusting the ratio of the number of feeder cells to the starting number of cells to be expanded. For example, in several embodiments, 1:1 ratios are used, while in additional embodiments, can range from about: 1:2, 1:5, 1:10, 1:20, 1:50, 1:100, 1:1,000, 1:10,000, 1:50,000, 1:100,000, 100,000:1, 50,000:1, 10,000:1, 1,000:1, 100:1, 50:1, 20:1, 10:1, 5:1, 2:1, and any ratio in between those listed, including endpoints. In several embodiments, the ratio at the inception of expansion is maintained at the same approximate ratio for each subsequent pulse. However, in several embodiments, the ratio is altered over time, for example to adjust the rate of expansion of the cells, whether a faster or slower expansion rate is desired.

In several embodiments, after the first period of expansion, the expanded cells (e.g., NK cells) are transduced with an engineered construct, such as a chimeric antigen receptor. Any variety of chimeric antigen receptor can be expressed in the engineered cells, such as NK cells, including those described in International PCT Application PCT/US2018/024650, PCT/IB2019/000141, PCT/IB2019/000181, and/or PCT/US2020/020824, PCT/US2020/035752, U.S. Provisional Application No. 62/924,967, 62/960,285, and/or 63/038,645, each of which is incorporated in its entirety by reference herein.

After viral transduction, the engineered cells are cultured for a second period of time, for example about 6 hours, about 12 hours, about 18 hours, about 24 hours, about 2 days, about 3 days, about 4 days, about 5 days, about 6 days, about 7 days, about 8 days, about 9 days, about 10 days, about 11 days, about 12 days, about 13 days, about 14 days, or for any time between those listed, including endpoints. It shall be noted that certain data presented herein relates to viral expression of a chimeric receptor complex expressing an NKG2D ligand binding domain (e.g., NKX101) or CD19 (e.g., NK19-1 or NKX101). However, any suitable chimeric receptor or chimeric antigen receptor can be used.

Supplementation of the media with one or more stimulatory agents, such as IL12 and/or IL18 can occur at any time during the culturing process. For example, one or more stimulatory agents can be added at the inception of culturing, for example at time point zero (e.g., inception of culture). The agent, or agents, can be added a second, third, fourth, fifth, or more times. Subsequent additions may, or may not, be at the same concentration as a prior addition. The interval between multiple additions can vary, for example a time interval of about 12 hours, about 24 hours, about 36 hours, about 48 hours, about 72 hours, or longer, and any time therebetween, including endpoints.

If multiple additions of a stimulatory agent are used, the concentrations of a first supplemental addition can be at the same or a different concentration than the second (and/or any supplemental addition). For example, in several embodiments, the addition of a stimulatory agent over multiple time points can ramp up, ramp down, stay constant, or vary across multiple, non-equivalent concentrations.

In several embodiments, certain ratios of feeder cells to cells to be expanded are used. For example, in several embodiments a feeder cell : “target” cell ratio of about 5:1 is used. In several embodiments, 1:1 ratios are used, while in additional embodiments, can range from about: 1:10, 1:20, 1:50, 1:100, 1:1,000, 1:10,000, 1:50,000, 1:100,000, 100,000:1, 50,000:1, 10,000:1, 1,000:1, 100:1, 50:1, 20:1, 10:1, and any ratio in between those listed, including endpoints.

### EXAMPLES

The materials and methods disclosed in the Examples are non-limiting examples of materials and methods (including reagents and conditions) applicable to various embodiments provided in the present application.

### Example 1—Initial Assessment of Expansion Conditions

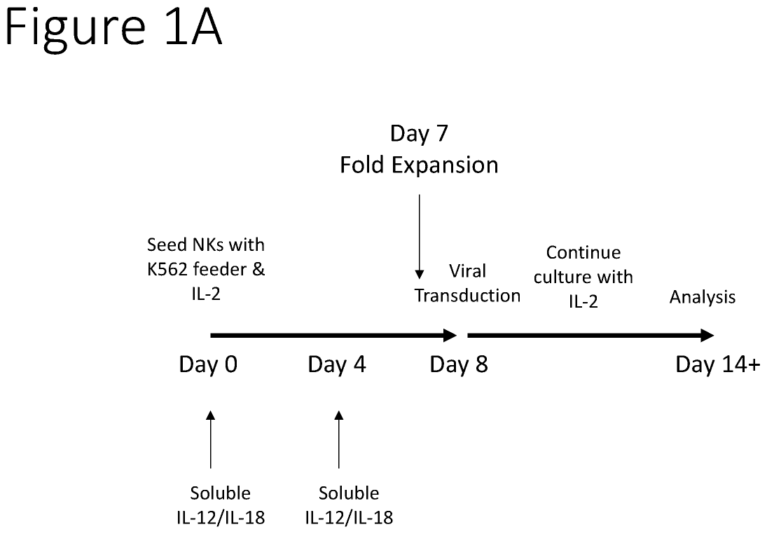

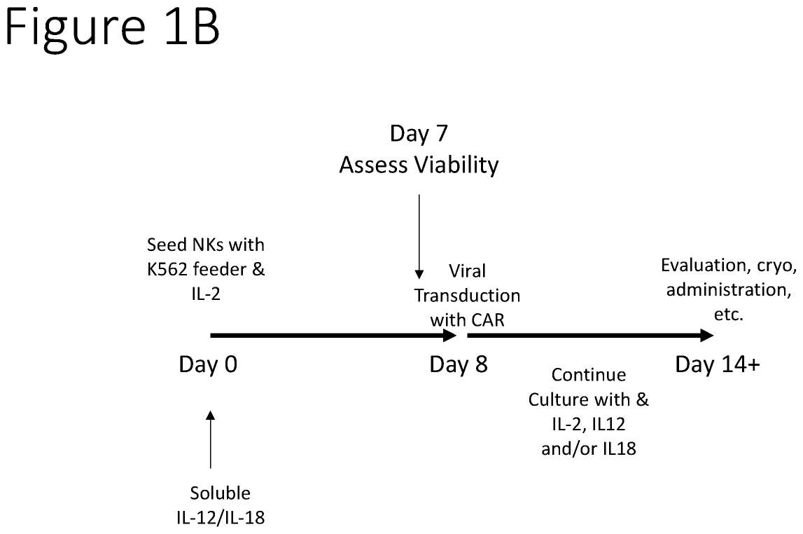

FIG. 1A shows a non-limiting example of an expansion process. In this example, stimulatory cytokines are added on day 0 and the same dose is added again at day 4, which was used for certain embodiments discussed herein. FIG. 1B represents a non-limiting embodiment of a single dose process, which was used for certain embodiments discussed herein.

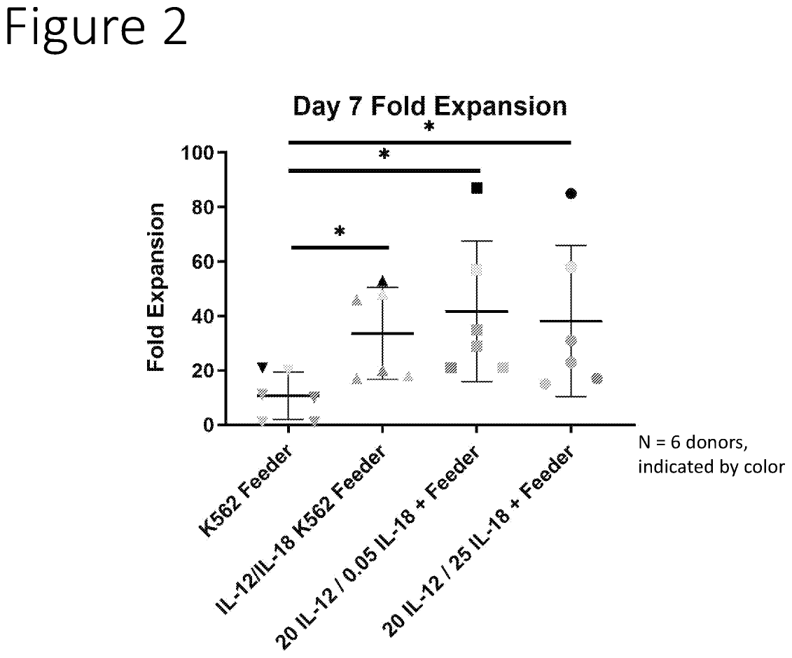

FIG. 2 shows data related to the fold expansion of the NK cells using various methods. The left-most data set shows expansion of NK cells using K562 (expressing mbIL15 and 4-1BBL) feeder cells alone, while each of the three data sets to the right show the increased fold expansion when supplementing the media with IL12 and IL18 at various concentrations. The presence of supplemental IL12 and IL18 at any amount resulted in a significant increase in expansion of NK cells, thereby demonstrating that additional stimulatory agents can enhance NK cell expansion.

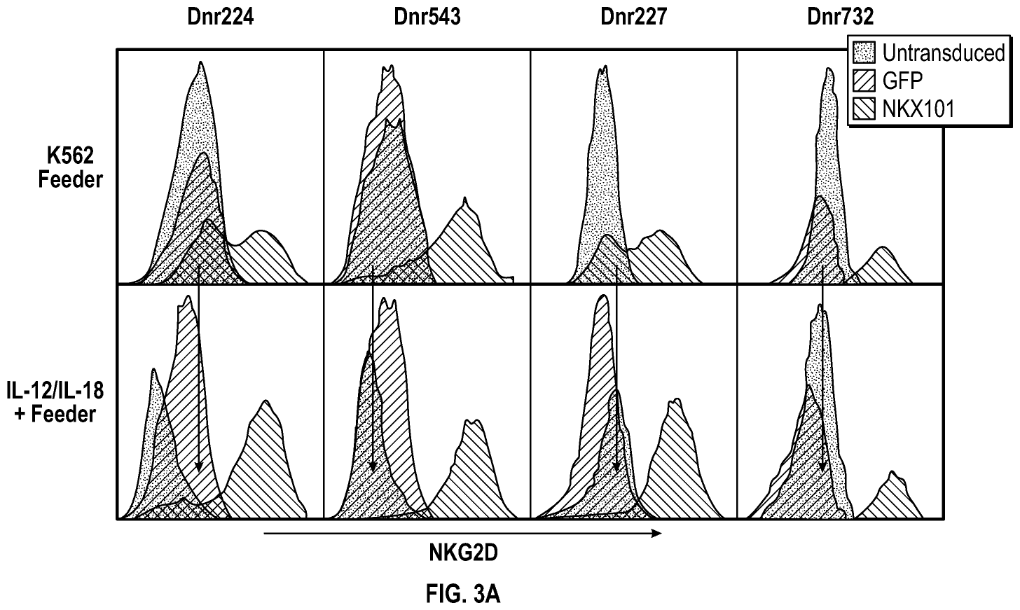

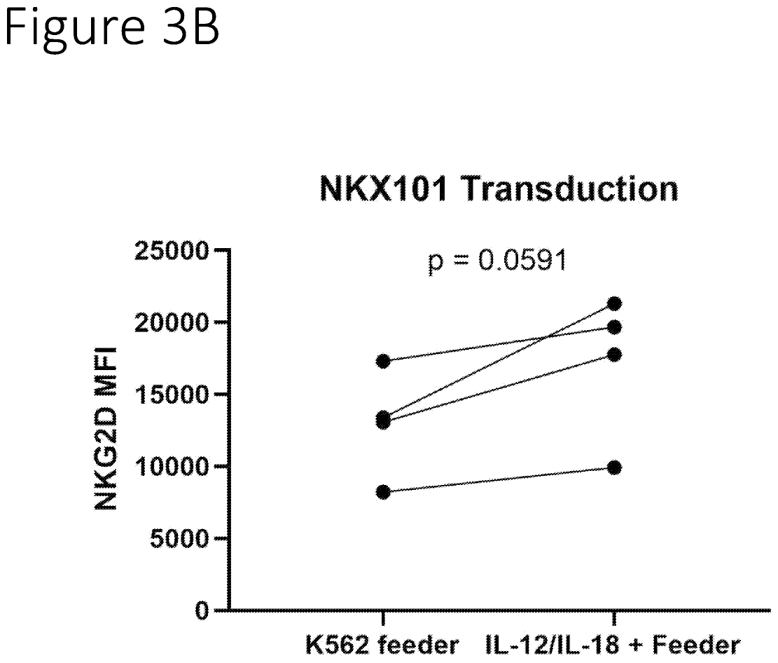

FIG. 3A shows flow cytometry data related to the expression of NKG2D in NK cells from four different donors, expanded either with K562 cells alone (top row) or with IL12/18 supplementation. As can be seen from the increased height of the right-shifted curve (which relates to cells transduced with NKX101), there is greater expression of NKG2D. The designation of NKX101 refers to an engineered NK cell that expresses a truncated NKG2D extracellular domain capable of binding ligands of the NKG2D receptor. In several embodiments the truncated NKG2D domain is coupled to a CD8alpha hinge and CD8alpha TM domain. In several embodiments, the truncated NKG2D domain is coupled to an OX40 co-stimulatory domain and a CD3zeta signaling domain. In several embodiments, the construct further comprises membrane bound IL15. In several embodiments, the NKX101 has the nucleotide sequence of SEQ ID NO: 1 or the amino acid sequence set forth in SEQ ID NO: 2. Further supporting the enhanced expression of NKG2D is FIG. 3A, in which the greater mean fluorescence intensity (MFI) when using supplemental soluble IL12/18 demonstrates greater presence of NKG2D on a given cell. Thus, not only does supplementing a feeder cell with soluble IL12/18 enhance expansion of NK cells, but it also improves the expression of chimeric receptors by those NK cells. This is an unexpected benefit, as the greater NK cell number now expresses greater amounts of a receptor that will target an undesired cell, such as a tumor.

Other receptors can be used to target NK cells to tumors. For example, in several embodiments the receptor is a chimeric antigen receptor targeting CD19 on tumor cells. In several embodiments, the anti-CD19 CAR comprises an scFv that binds to CD19 (for example an FMC63 scFv or variant thereof) coupled to an OX40 costimulatory domain and a CD3zeta signaling domain. In several embodiments, a nucleic acid sequence encoding the CAR further encodes IL15. In several embodiments, the IL15 is configured to be expressed by a host cell (e.g., an NK cell or a T cell) in a membrane-bound form. In several embodiments, the CAR is encoded by a nucleotide sequence having at least 95%, 97%, 98%, 99% or more sequence identity to the sequence of SEQ ID NO: 3, 5, 7, 9, 11, 13, 15, 17, 19, 21, 23, 25, or 27. In several embodiments, the CAR is has an amino acid sequence having at least 95%, 97%, 98%, 99% or more sequence identity to the sequence of SEQ ID NO: 4, 6, 8, 10, 12, 14, 16, 18, 20, 22, 24, 26, or 28. In several embodiments, the CAR employs a humanized anti-CD19 binder.

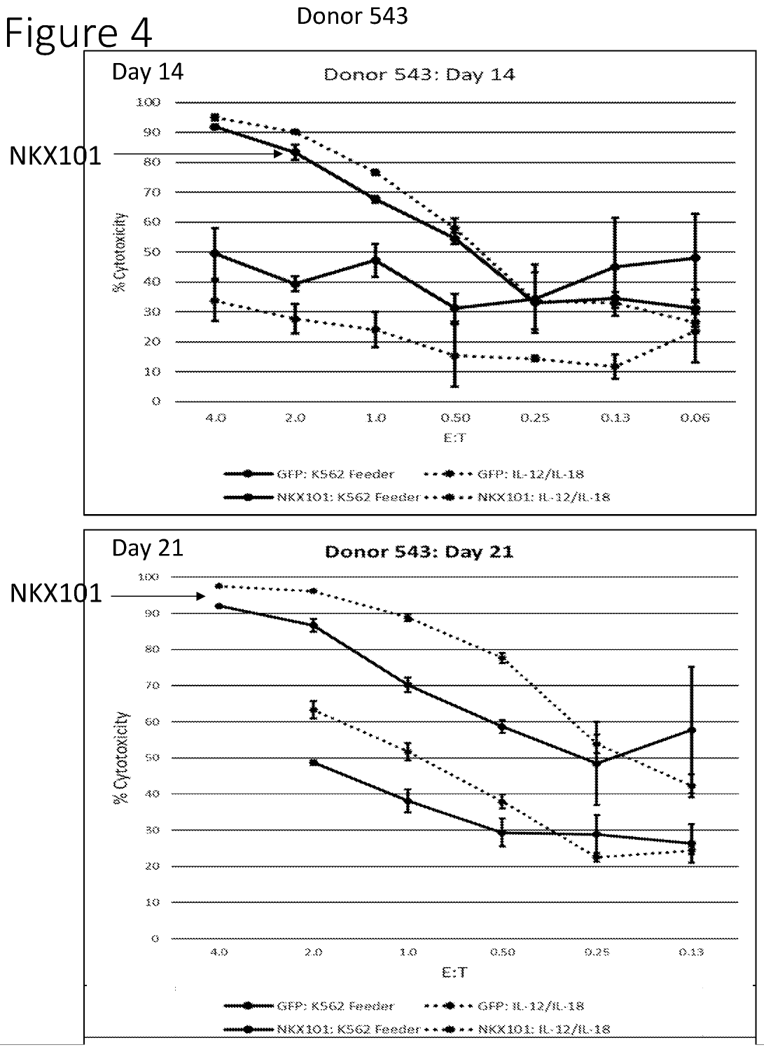

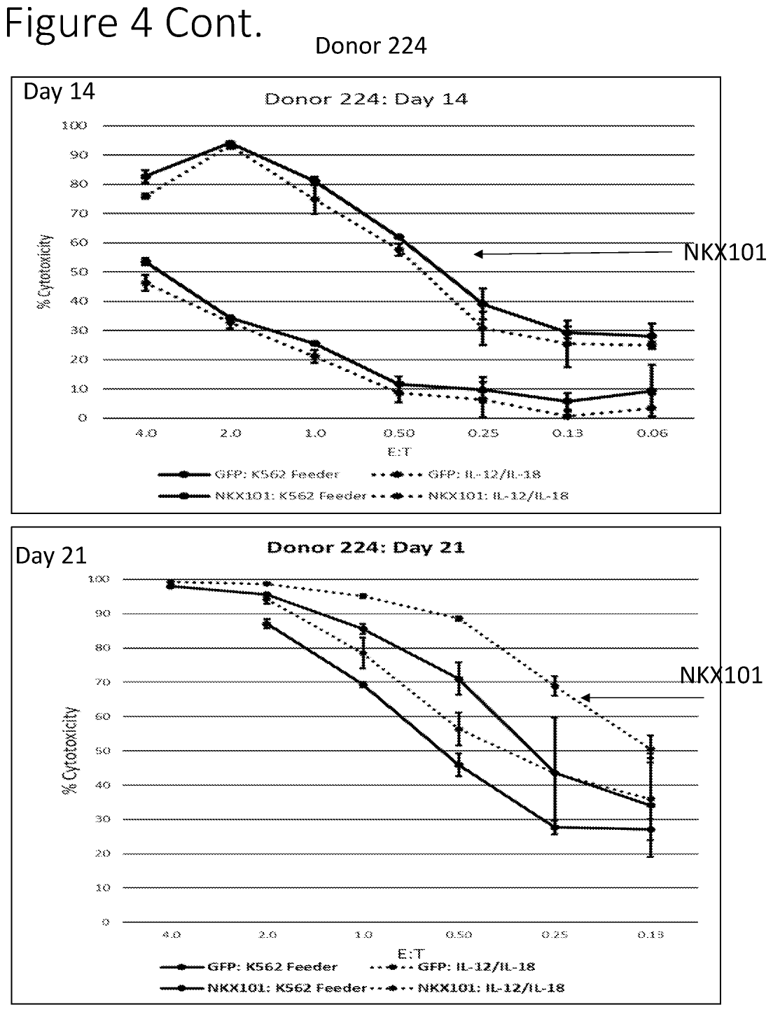

FIG. 4 depicts data in which the use of supplemental soluble IL12/18 when expanding NK cells actually leads to enhanced cytotoxicity of those expanded NK cells. FIG. 4 shows data from two different donors, at two time points, 14 days, and 21 days post viral transduction. Culture conditions used to expand the NK cells were either with the use of soluble IL12/18 (dashed lines) or K562 (expressing 4-1 BBL and mbIL15) alone (solid lines). GFP transduced cells were used as controls—NKX101 curves are indicated by arrows on FIG. 4. As the data indicate, relative to expansion on K562 cells alone, the use of IL12/18 enhances NK cell cytotoxicity at 21 days post-transduction (lower panels). While the effect at 14 days was limited in this specific experiment, in several embodiments, perhaps depending on donor and/or specific IL concentrations, in several embodiments, enhanced cytotoxicity is achieved at earlier time points, such as 14, 15, 16, 17, 18, 19 20, 21, 22, or 23 days post viral transduction. Regardless of the time, it is unexpected that the use of soluble interleukins during the expansion process can significantly enhance the cytotoxicity of the expanded cells.

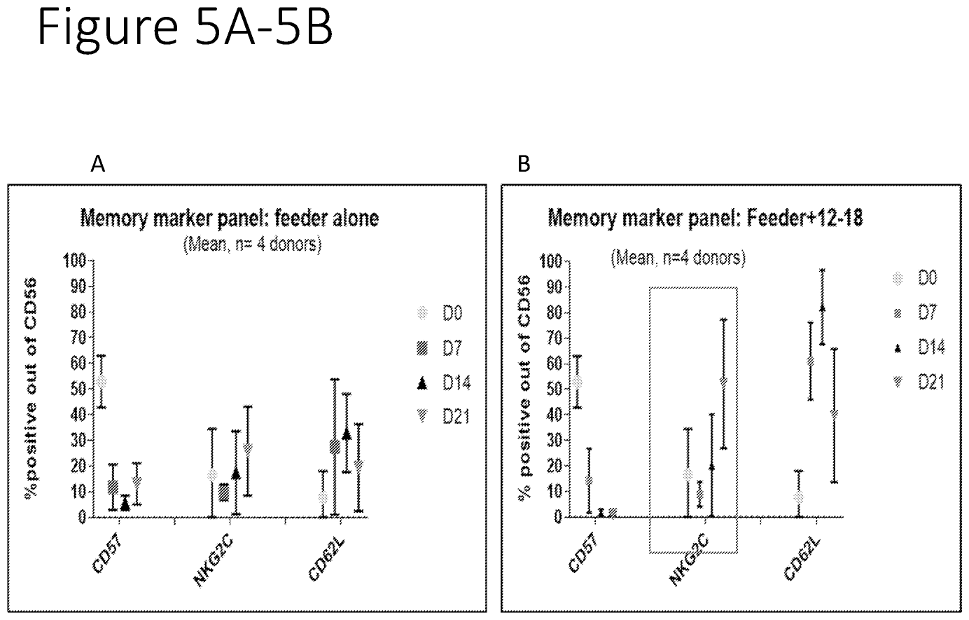

In several embodiments, the increased cytotoxicity of the engineered NK cells is, at least in part, due to the cells moving towards a specific phenotype. FIGS. 5A and 5B depict data related to certain markers related to NK cell memory over time. FIG. 5A shows the expression of CD57, NKG2C and CD62L in NK cells expanded on feeder cells alone, while FIG. 5B shows the use of feeder cells plus soluble IL12/18. NKG2C expression was elevated at Day 21 in those NK cells expanded with IL12/18. NKG2C is a marker of cytokine-induced NK cell memory. Increased CD67L was also observed in the later time points with NK cells expanded using soluble IL12/18. CD67L is associated with increased lymphocyte extravasation (evidence of increased cell activity). Taken together, these data suggest that the use of soluble interleukins during NK cell expansion have the capacity to set in motion different signaling pathways that are associated with NK cell memory for antigens and enhanced cytotoxicity against cells bearing those antigens.

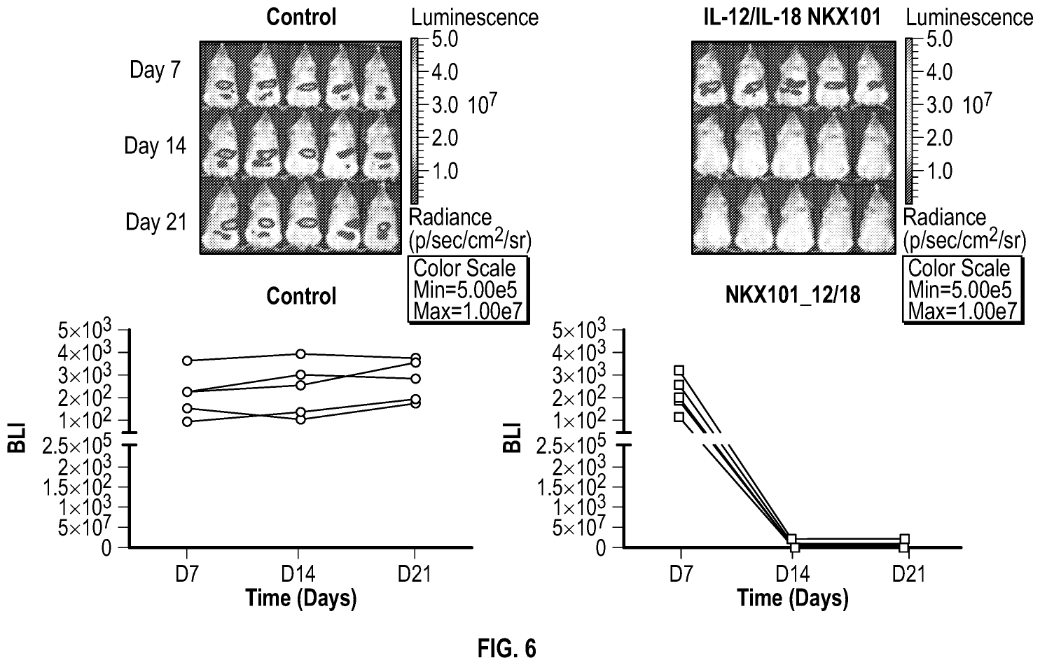

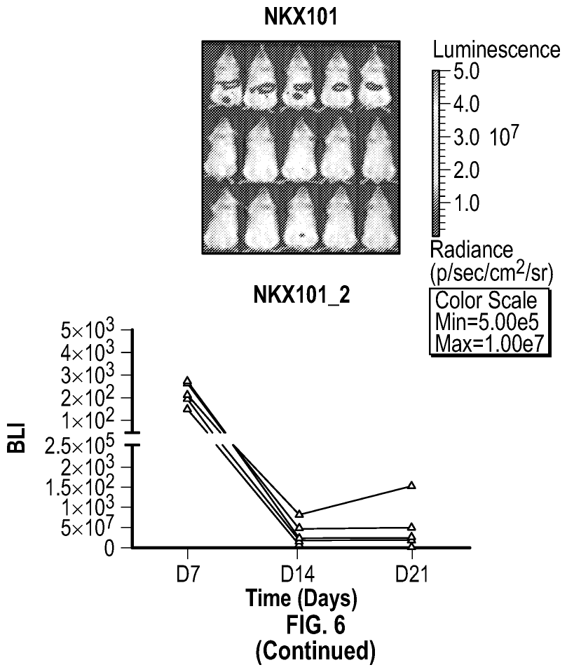

FIG. 6 depicts in vivo data related to the anti-tumor effect of NK cells expressing NKX101 when the underlying NK cells were expanded using K562 cells alone, vs. supplanting the expansion media with soluble IL12/18. The animal model involves dosing mice with 4×10⁶ SNU499 hepatocellular carcinoma cells (intraperitoneally) at Day 0, followed by 3×10⁶ NK cells expressing NKX101, having been expanded with, or without IL12/18 supplementing the expansion media (or control). As shown in the left panels, control mice have significant tumor burden as early as day 7, with tumor signal being present, and modestly increased in some mice, on days 14 and 21. In vivo bioluminescent imaging (BLI) is shown below the images. The right panel shows the experiment done with NK cells expressing NKX101. As shown in the images, tumor burden was present at day 7, but largely non-detectable by day 14, and maintained as such by day 21. In the center panel, the experimental images are shown for NK cells expressing NKX101, the NK cells having been expanded using soluble IL12/18. The effect on tumor burden was at least as effective as with NKX101 cells (“standard” expansion), although the significant degree of NKX101 efficacy can make the improved effect with IL12/18 difficult to detect. Nevertheless, according to several embodiments disclosed herein, the use of soluble IL12/18 to supplement NK cell expansion media results not only in enhanced expansion, but also enhanced chimeric receptor expression and enhanced cytotoxicity.

### Example 2—Further Assessments of Expansion and Efficacy

As discussed above, in several embodiments disclosed herein, one or more soluble stimulating factors are used to enhance the expansion and/or cytotoxicity of engineered immune cells, such as NK cells, T cells, or combinations thereof. The experiments conducted for the present example were performed in order to assess the efficacy of various concentrations of selected stimulators molecules as compared to an established expansion system. While other stimulating agents can be used, depending on the embodiment, this example employed soluble interleukin 12 and soluble interleukin 18. These cytokines were added (in the various concentrations described below) and the resultant expanded cells were compared to cells expanded using K562 cells modified to express membrane-bound interleukin 15 and 4-1 BBL (described more fully in U.S. Pat. Nos. 7,435,596 and 8,026,097 the entire contents of each of which is incorporated in its entirety by reference herein). Expanded cells were assessed with respect to proliferation, cytokine secretion, cytotoxicity and phenotype.

Experiments were set up using NK cells from multiple donors which were expanded using various conditions. One group of NK cells was expanded on mbIL15-expressing feeder cells (K562/4-1BBL/mbIL15). Another group of NK cells was expanded on mbIL15-expressing cells that were further modified to express IL12 and IL18 on the cell surface. Various culture conditions were used across the other groups, and a proliferation assays were performed to determine the effects of various concentrations of stimulatory cytokines. For example, one group of cells was exposed to a fixed concentration of IL12 (5 ng/mL) and varied concentrations of IL18. An additional group was exposed to another fixed concentration of IL12 (2.5 ng/mL) and varied concentrations of IL18. Note that those cultures that are exposed to IL12 and IL18 in soluble form were exposed to the dose of IL12/18 at day zero of culture (and again at day 4). As discussed above, the addition of soluble cytokines at day 0 and day 4 was used in the experiments generating the data shown in FIGS. 2-18 and FIGS. 23-24. The other experiments utilized exposure to the soluble cytokines at day 0 only.

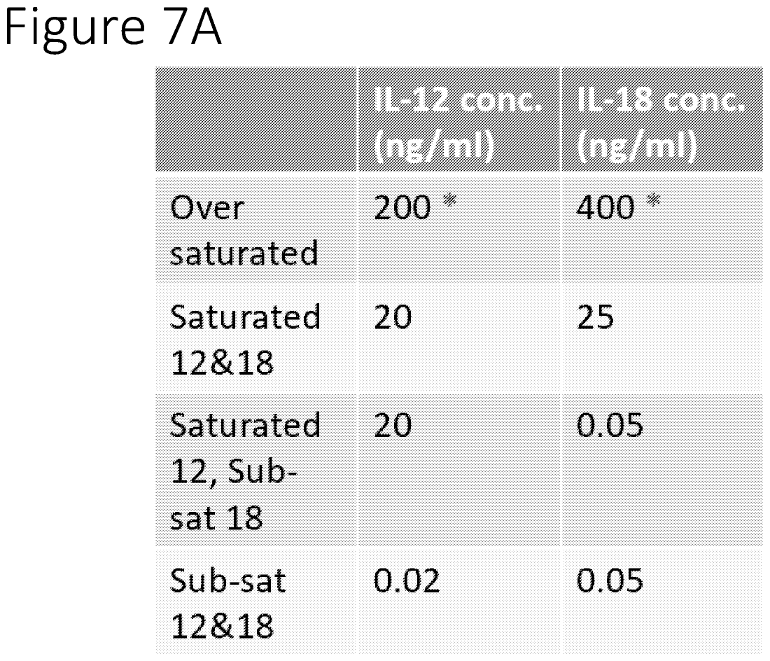

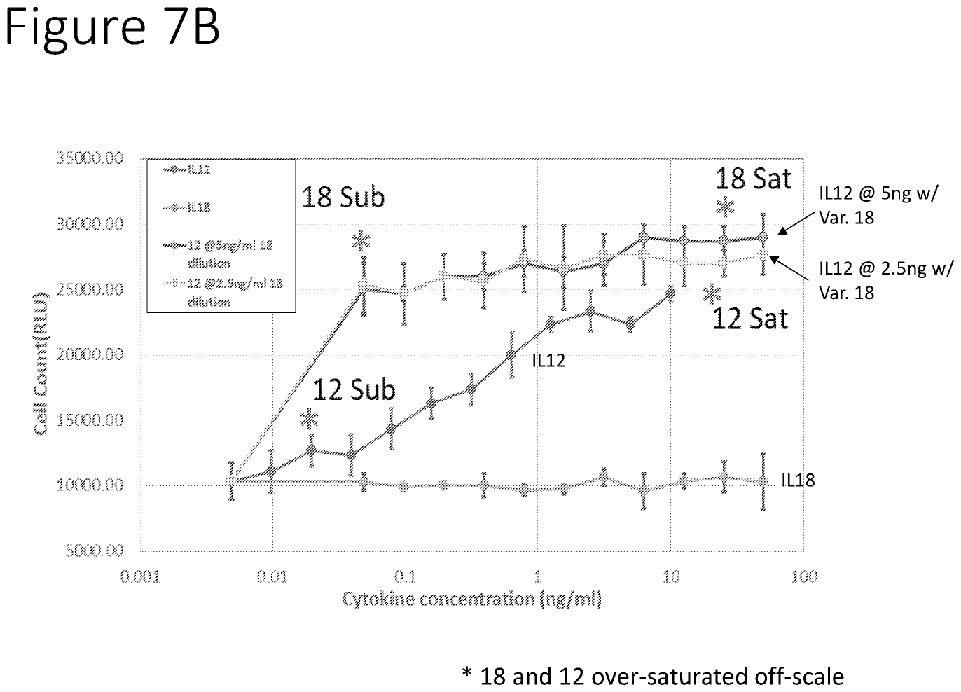

FIG. 7A a schematic table of various culture conditions used for expansion of NK cells. FIG. 7B shows data related to the cell count after 72 hours of exposure to the various conditions. As seen from the lower trace, the addition of IL18 alone, at any concentration, had limited impact on NK cell proliferation. In contrast, addition of IL12 alone increased NK cell proliferation in a dose-dependent manner. The combination of IL12 (either at 2.5 ng/mL or 5 ng/mL) with varied concentrations had further enhanced NK cell proliferation, suggesting a synergistic interaction between these two interleukins. The data for IL12 at 2.5 ng/mL and 5 ng/mL both demonstrate robust NK cell expansion, with near maximal levels achieved when IL18 was present at a concentration between about 0.1 and about 1 ng/mL. Addition of IL18 at higher concentrations was still able to positively enhance NK cell expansion, with the highest concentration of IL18 at 50 ng/mL in combination with IL12 at 5 ng/mL resulting in slightly enhanced expansion as compared to IL12 at 2.5 ng/mL. The data for expansion with oversaturated concentrations of IL12 or IL18 were off the scale and are not shown.

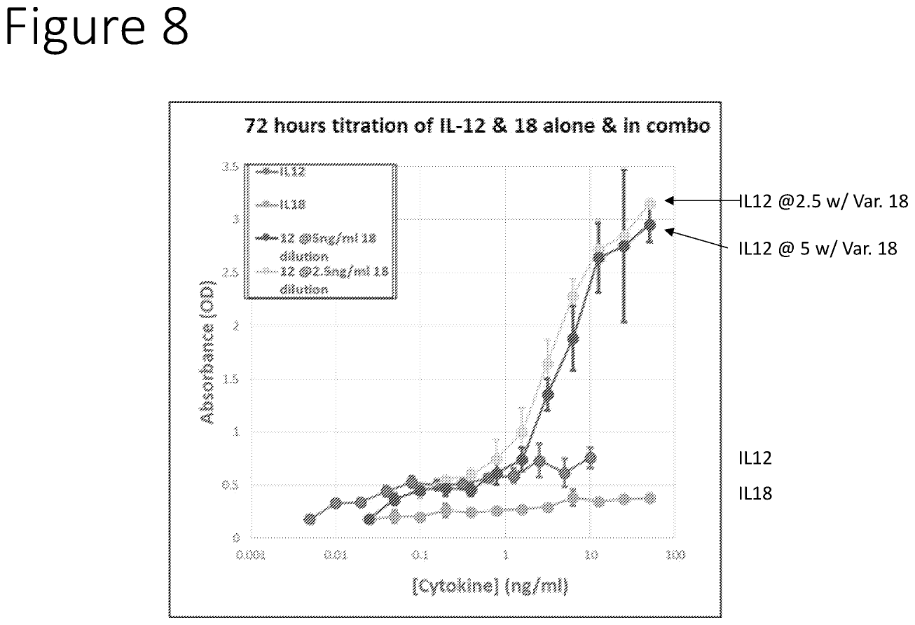

FIG. 8 shows data related to IFNg concentrations after 72 hours of culture with varied concentrations of either IL12 or IL18. The data plot represents the concentration of IFNg (as measured by absorbance during an ELISA assay) in relation to increasing concentrations of the selected interleukin. Similar to the proliferation data, addition of IL12 resulted in greater production of IFNg as compared to addition of IL18. That said, the addition of increasing concentrations of IL18 did result in increased IFNg production. IL12, on the other hand, resulted in greater IFNg production by the NK cells at nearly every concentration tested. As with proliferation, the combination of either concentration of IL12 with concentrations of IL18 of about 1 ng/mL (or greater) yielded enhanced IFNg production. The combination of IL12 (at either concentration) with IL18 at concentrations below about 0.5 ng/mL resulted in IFNg production similar to that achieved with IL12 alone. On the other hand, inclusion of IL18 at about 1 ng/mL or greater led to significantly enhanced IFNg production, again indicating a synergistic stimulation of the NK cells.

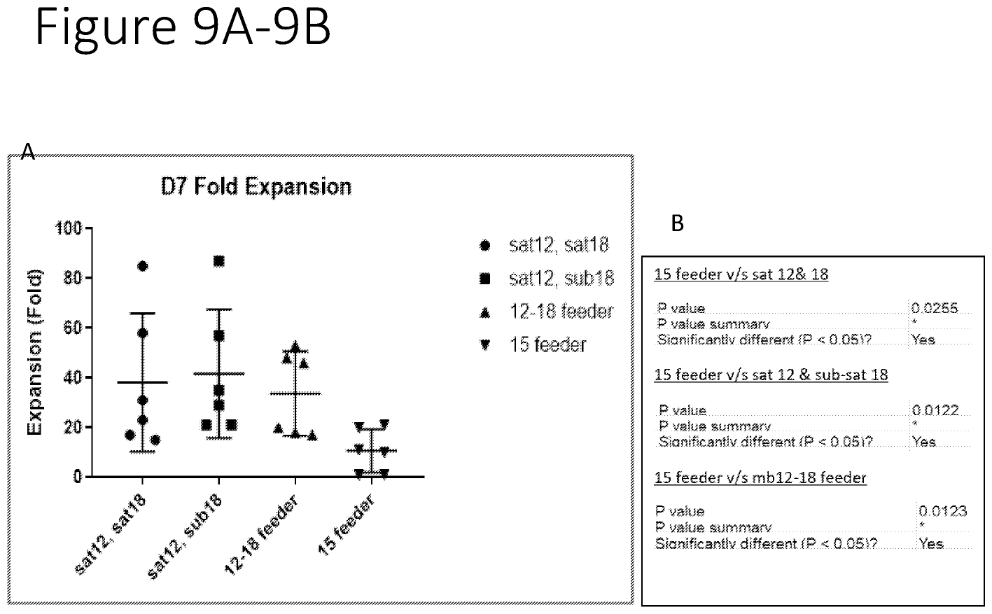

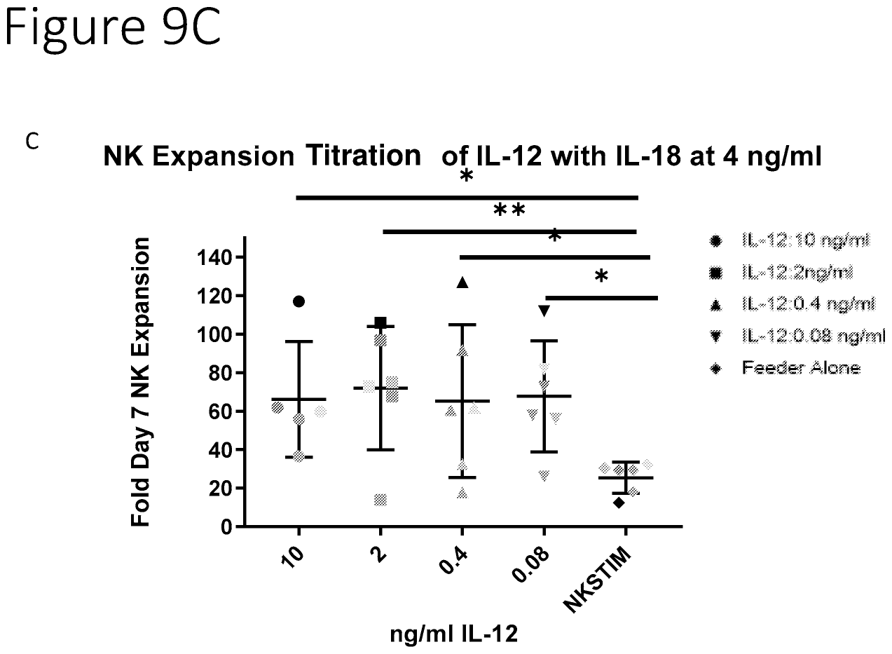

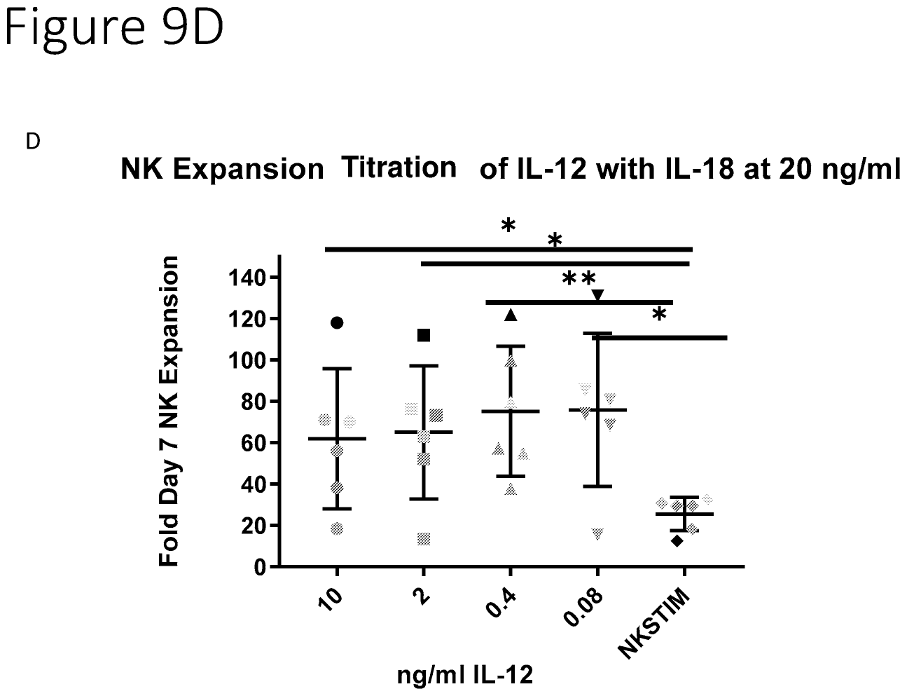

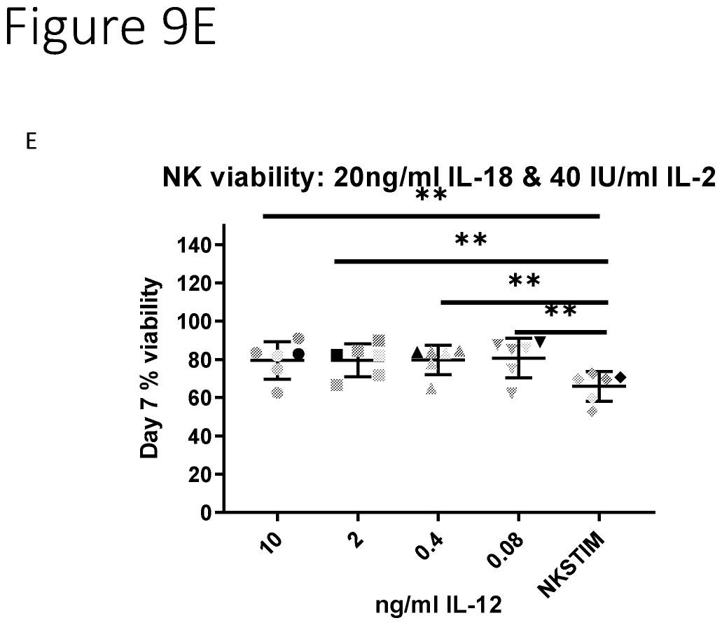

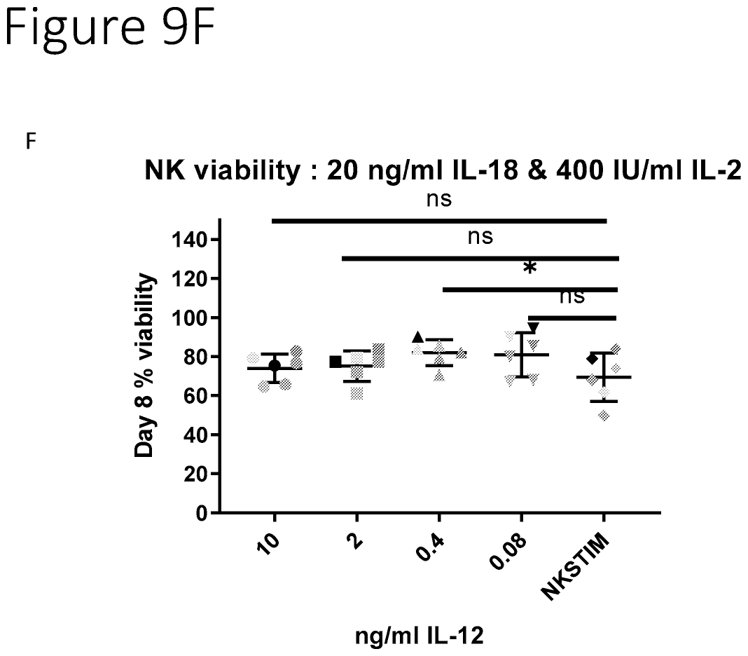

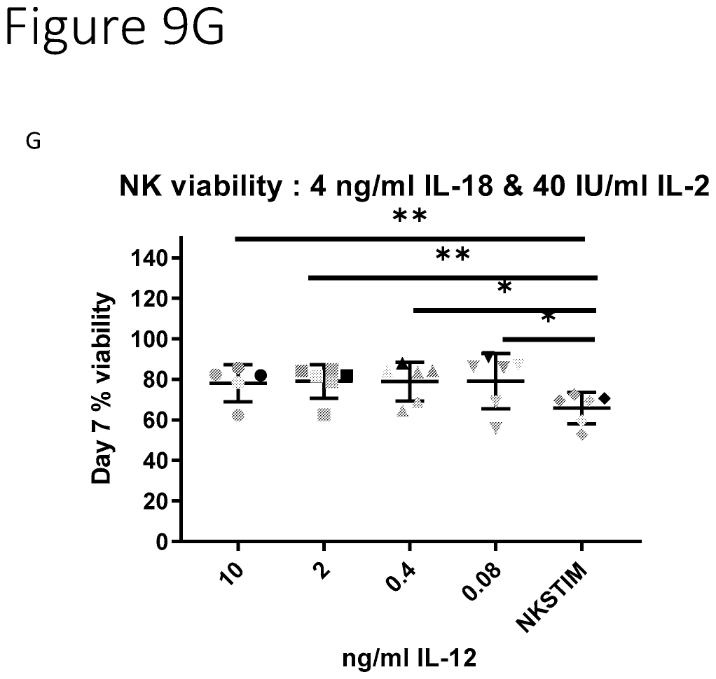

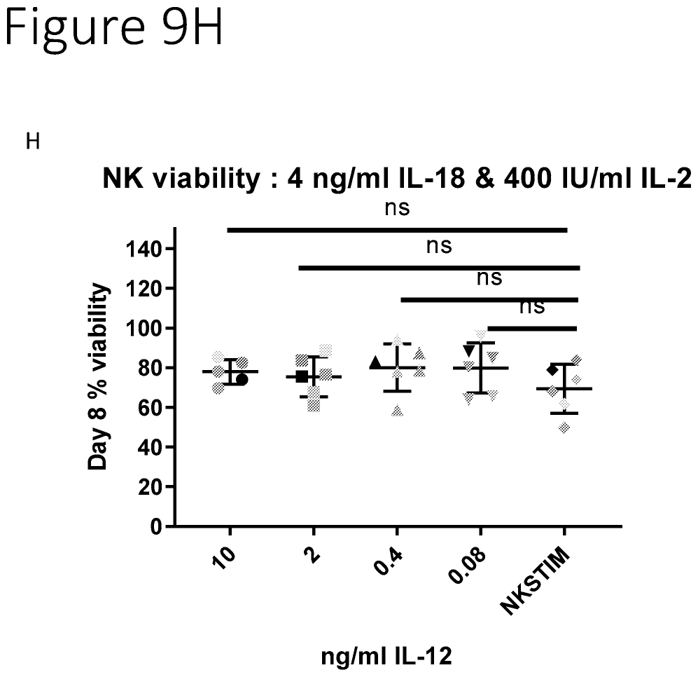

FIGS. 9A-9B shows data related the expansion of NK cells (un-transduced) after 7 days of expansion in the indicated culture conditions. A first group was expanded using saturated concentrations of both IL12 (20 ng/mL) and IL18 (25 ng/mL). A second group was expanded using saturated concentrations of IL12 (20 ng/mL) and sub-saturated concentrations of IL18 (0.05 ng/mL). A third group was expanded using feeder cells engineered to express membrane-bound forms of each of IL15, IL12 and IL18 (further details on this feeder cell line can be found in International Patent Application No. PCT/SG2018/0501387, which is incorporated by reference herein in its entirety). A fourth group, as a control, was expanded on an established feeder cell line (K562 cells expressing mbIL15 and 4-1BBL). FIG. 9A shows the calculated expansion data and FIG. 9B shows the statistical analysis. FIGS. 9C and 9D display data to specific titration curves and NK cell expansion. FIG. 9C shows data for various concentrations of IL12 with IL18 held constant at 4 ng/mL. FIG. 9D shows similar data with IL12 varied and IL18 at 20 ng/mL. Taken together, these data indicate that addition of IL12 and IL18, whether in soluble format or membrane bound on the feeder cells (such as K562 cells expressing mbIL15) yields significantly enhanced NK cell expansion. Interestingly, IL12 appears to be a primary driver of expansion, with its activity enhanced by inclusion of IL18, even at low concentrations (see, e.g., the similar expansion numbers for saturated and sub-saturated concentrations of IL18. These data indicated that combinations of IL12 and IL18 robustly enhance NK cell expansion.

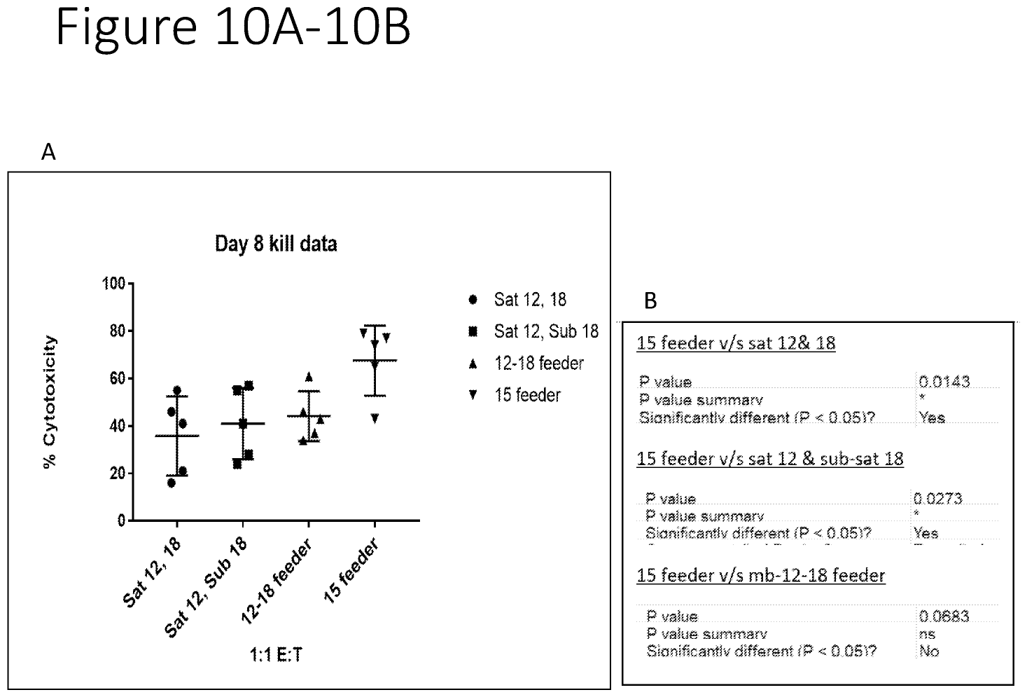

FIGS. 10A-10B show cytotoxicity data for the un-transduced NK cells after 8 days of expansion in the indicated conditions (and IL2 media supplementation at 40 IU/mL). Target cells were Reh acute lymphocytic leukemia (non-T; non-B) cells at a 1:1 effector target ratio. Regardless of culture conditions, all cells exhibited between about 40% and about 65% cytotoxicity. Cells expanded on mbIL15-expressing feeder cells without any IL12 or IL18 exhibited the highest degree of cytotoxicity, significantly more than either of the groups cultured in soluble IL12/IL18. Use of feeder cells with membrane-bound IL12 and IL18 exhibited greater degrees of cytotoxicity than those with soluble cytokines.

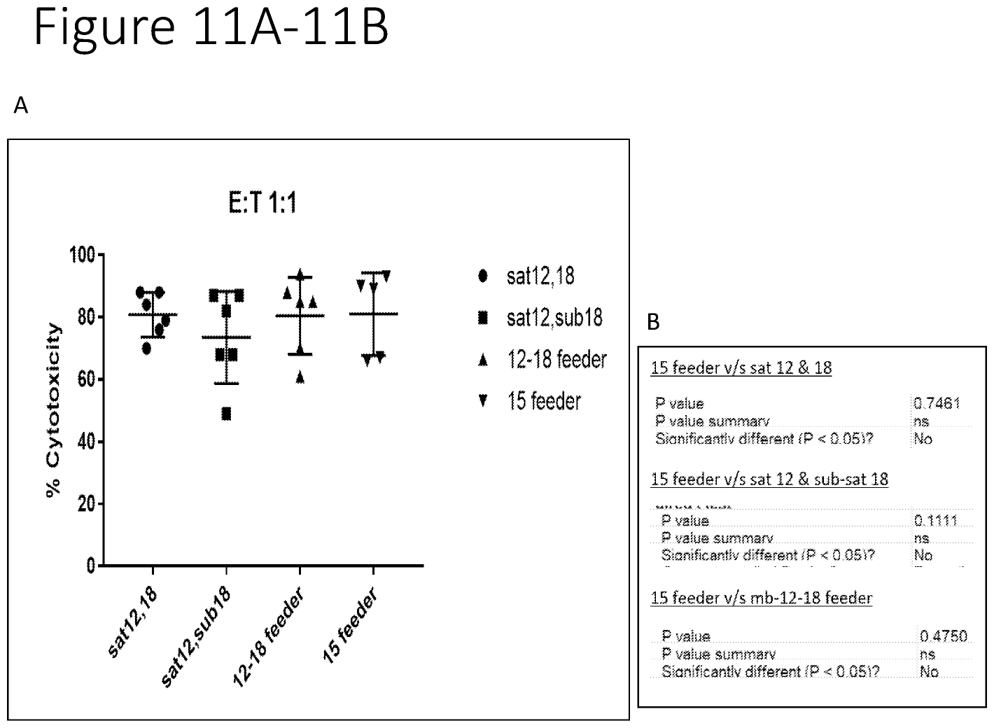

FIGS. 11A-11B show cytotoxicity data for un-transduced NK cells at day 15 of culture (IL2 concentrations of 400 IU/mL) against Reh cells at 1:1 effector target ratio. These data exhibit not only greater degrees of cytotoxicity across the groups tested, but limited differences between the groups. In other words, all groups show increased cytotoxicity to the degree that there is not a significant difference between the culture conditions. According to some embodiments, the use of IL12 and IL18 induces a pathway or signaling cascade that impacts expansion in the early portion of culture. In several embodiments, that pathway or cascade (or pathways/cascades) has a delayed impact on enhanced cytotoxicity. In several embodiments, the use of certain stimulating factors induce a phenotypic change in the NK cells, such as a memory-like phenotype, that primes the NK cells to exert cytotoxic effects against a target cell. In several embodiments, the induction of that phenotypic change can take 1-2, 3-4, 5-6, 7-8 or more days to be recognized, depending on the characteristic of the NK cell being evaluated.

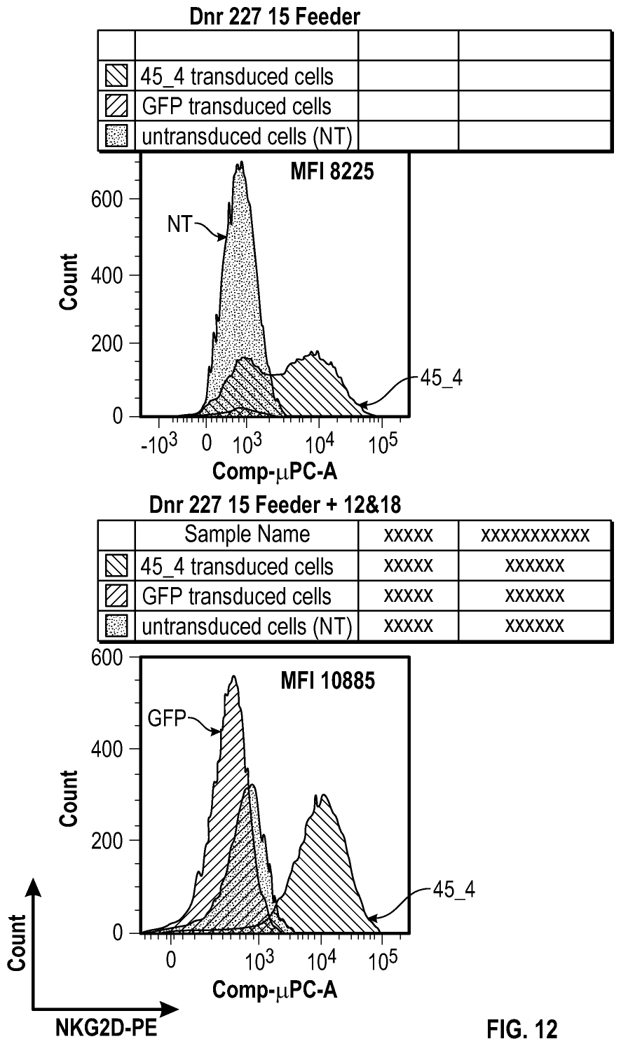

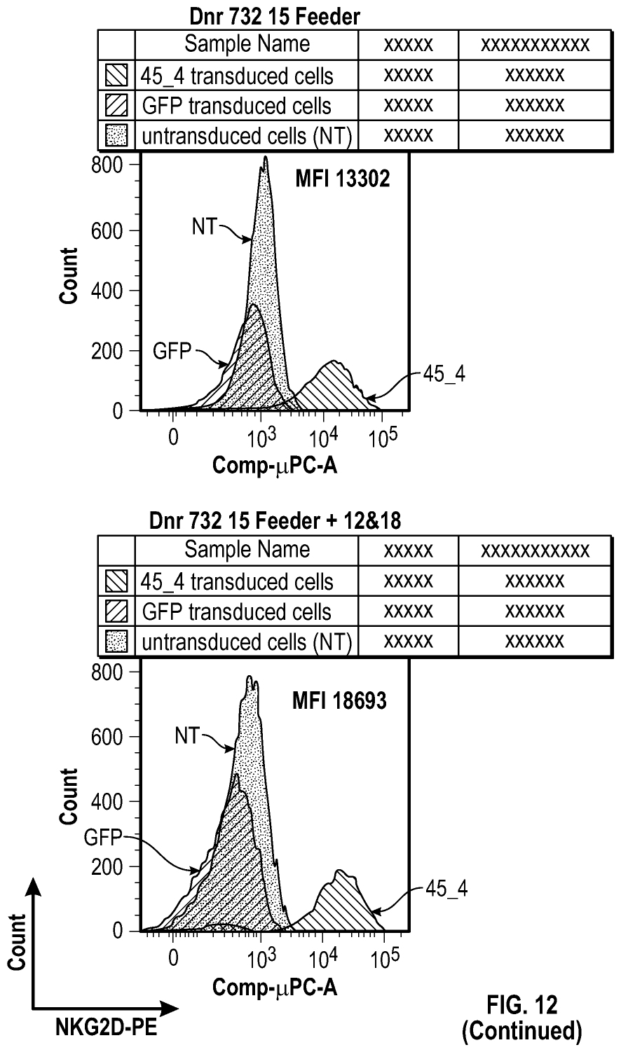

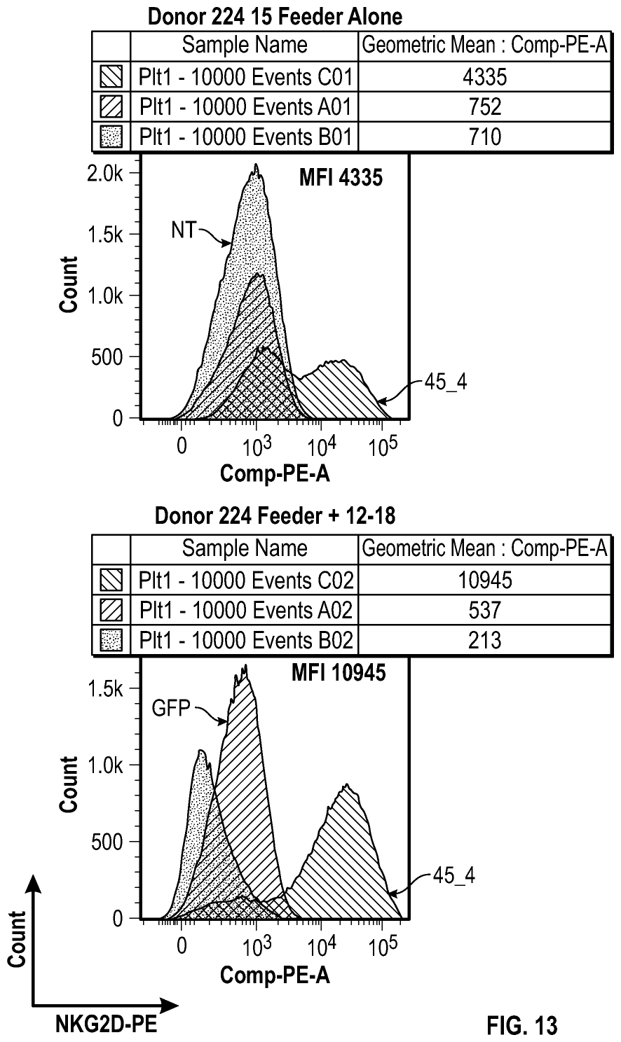

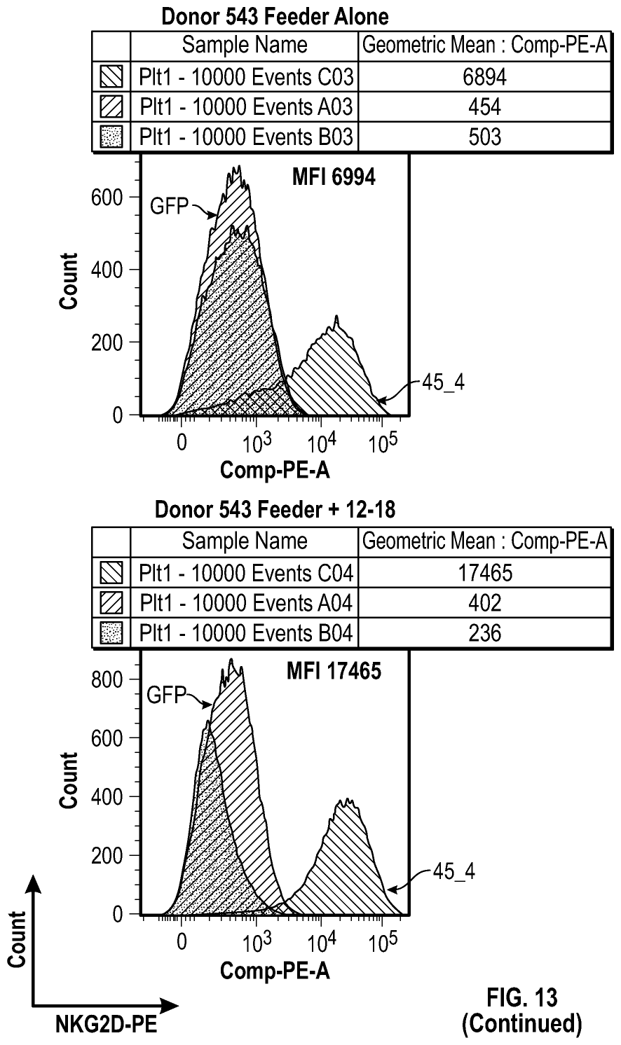

While the experiments above were performed with un-transduced NK cells, they demonstrate that inclusion of IL12 and IL18, at various concentrations can enhance expansion and cytotoxicity of the NK cells. Further experiments were undertaken with NK cells transduced with a chimeric receptor (as compared to GFP-transduced cells or un-transduced (NT) NK cells). As a non-limiting example the chimeric receptor employed comprises a truncated NKG2D domain is coupled to a CD8alpha hinge and CD8alpha TM domain an OX40 co-stimulatory domain, a CD3zeta signaling domain, and membrane bound IL15. FIG. 12 shows flow cytometry data evaluating the expression of the chimeric receptor (indicated as 45_4) on NK cells from various donors that were cultured under various conditions. In the left column of FIG. 12, data is shown for NK cells cultured on mbIL15 expressing feeder cells for two donors (227 on top, 732 on bottom). The curve identified as “45_4” shows greater expression of NKG2D (as expected for those cells being transduced with the NKG2D-containing chimeric receptor). The right column shows the expression results for NK cells cultured on mbIL15-expressing feeder cells with soluble IL12 and soluble IL18 added to the media at day 0 at 20 ng/mL and 25 ng/mL, respectively. FIG. 13 shows corresponding data for two additional donors. As can be seen from the MFI data in both FIG. 12 and FIG. 13, use of IL12 and IL18 resulted in enhanced NKG2D expression, further supporting the prior data that certain stimulating factors can robustly drive NK cell expansion. These data also confirm that use of stimulatory molecules, such as IL12 and IL18 are compatible with transduced NK cells.

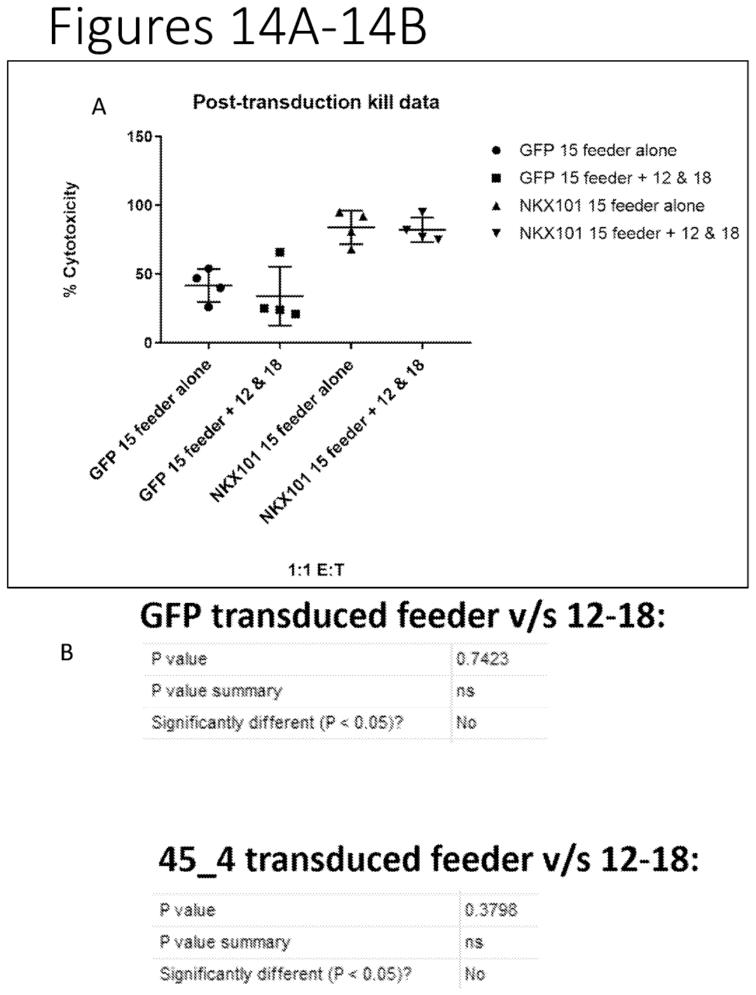

Having confirmed that stimulatory cytokines enhance the expansion of transduced NK cells, cytotoxicity was evaluated. FIGS. 14A and 14B show data related to cytotoxicity of NK cells transduced with the indicated constructs and expanded using the indicated culture conditions. Groups were: GFP-transduced NK cells grown on mbIL-15-expressing feeder cells; GFP-transduced NK cells grown on mbIL-15-expressing feeder cells and exposed to IL12 and IL18, NKX101-transduced NK cells grown on mbIL-15-expressing feeder cells and NKX101-transduced NK cells grown on mbIL-15-expressing feeder cells and exposed to IL12 and IL18. Target cells were Reh cells at 1:1 E:T ratio. The cytotoxicity was evaluated at Day 13 post-expansion using cells from four different donors. As shown, both GFP-transduced and NKX101-tranduced NK cells exhibited cytotoxicity, with NKX101-expressing cells showing greater effects against the target cells. No significant differences were detected based on the expansion culture conditions used (see 14B).

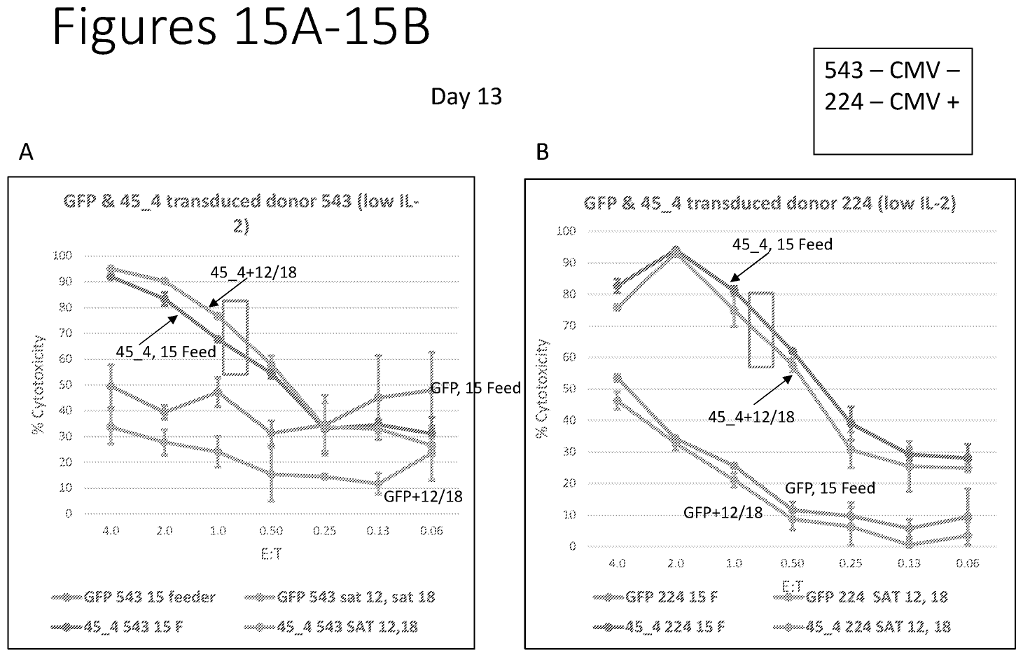

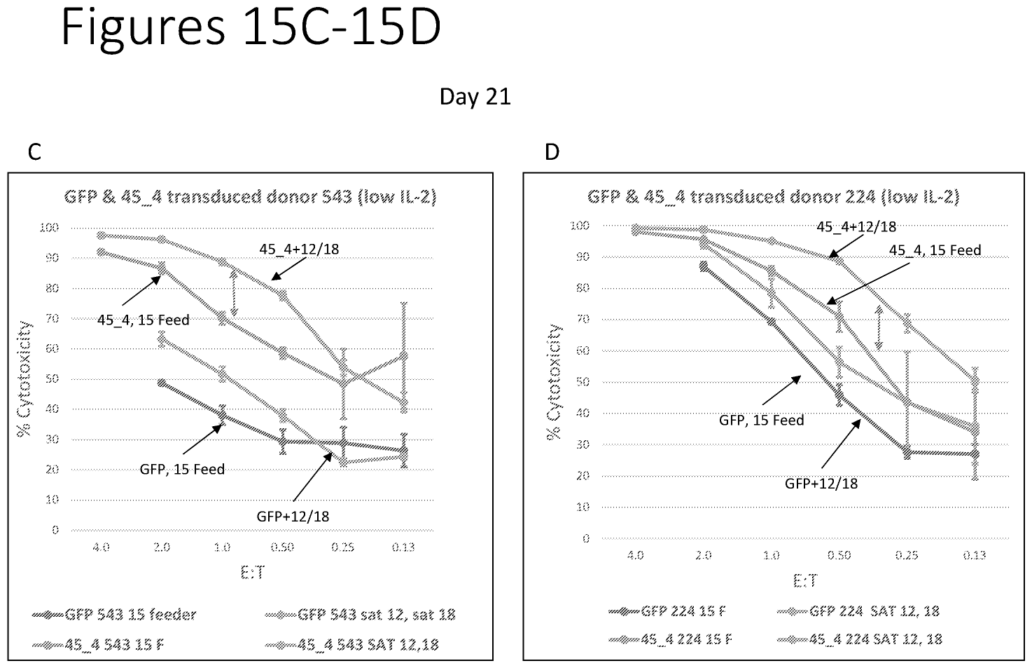

FIGS. 15A-15B show additional cytotoxicity data from two donors where different E:T ratios were tested. These data show a pattern consistent with that shown in FIG. 14. FIG. 15A shows data for the four culture conditions for a first donor, and 15B shows the corresponding data for a second donor. Note that donor 543 (FIG. 15A) was negative for cytomegalovirus and donor 224 (15B) was positive for CMV. CMV positive individuals have a subpopulation of NK cells that have a memory-like phenotype, meaning that they are characterized by a more rapid response to target cells. The data in 15A-15B was collected at day 13 post-expansion. These curves are similar to the data above and at this relatively early time point, the presence or absence of IL12/IL18 has a limited effect on the cytotoxicity induced by NK cells. FIGS. 15C and 15D show data from the same donors/conditions, but at 21 day post-expansion. Notably, the use of IL12/IL18 results in enhanced cytotoxicity against the target cells at most E:T ratios tested. These data are consistent with those discussed above for the un-transduced NK cells, in that there is a delay in the induction of enhanced cytotoxicity, but it is detectable at later time points. As discussed above, this effect may be due to the time required to induce a phenotypic change in the NK cells.

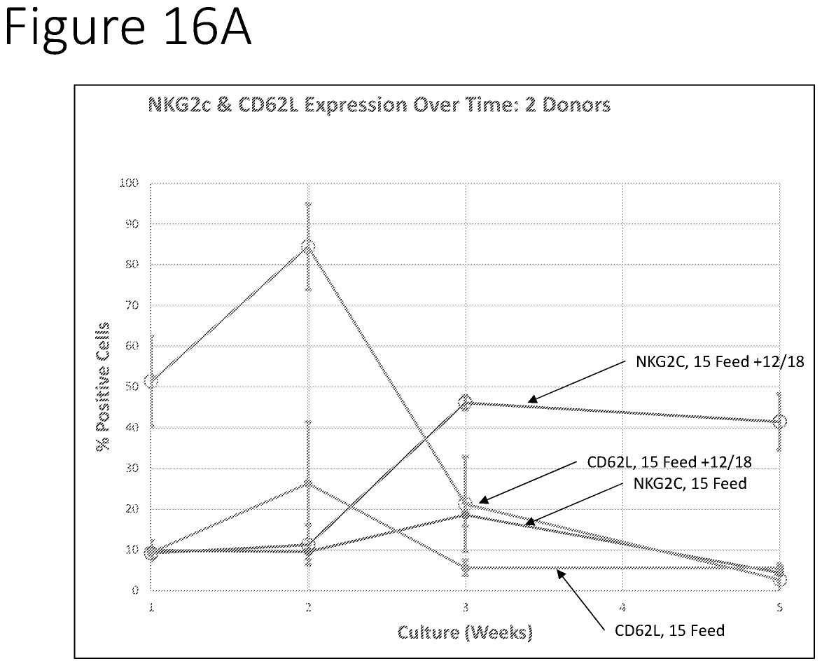

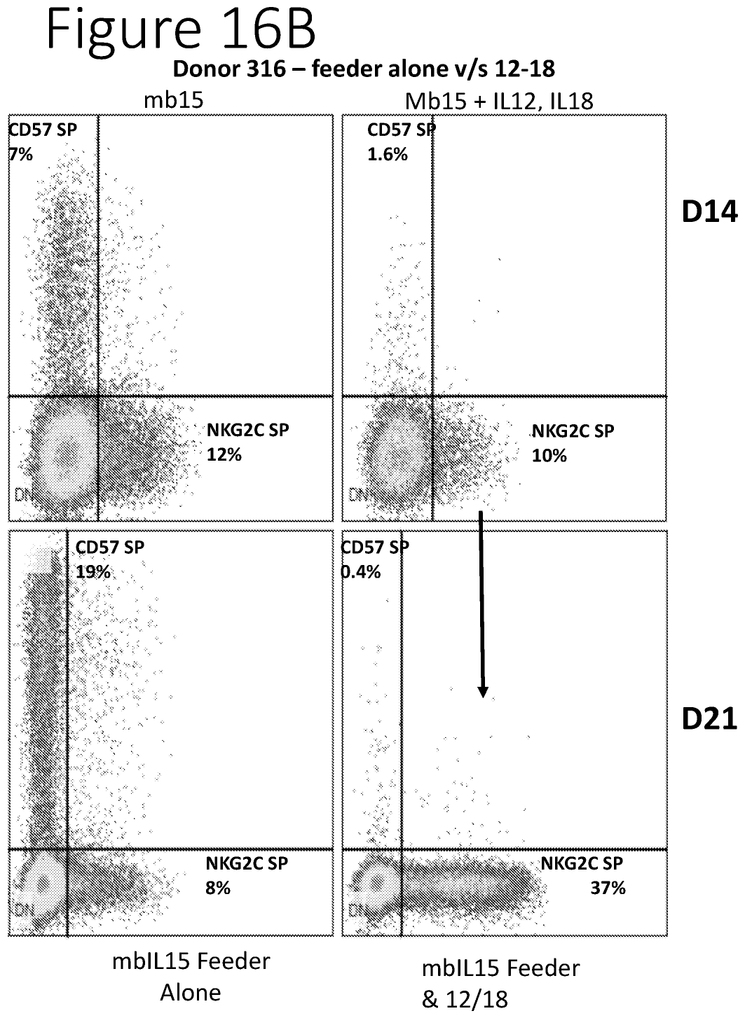

FIGS. 16A-16B relate to the evaluation of the phenotype of NK cells cultured in different conditions over time. FIG. 16A shows the expression levels of NKG2C and CD62L (L-selectin) over 5 weeks of culture under the indicated conditions. Neither CD62L or NKG2C expression levels varied significantly over the 5 weeks of culture when using mbIL15-expressing feeder cells. In contrast, however, use of those feeder cells and supplementing the media with IL12 and IL18 at day 0 had significant impact on the expression of both NKG2C and CD62L. CD62L was initially present on about 50% of the NK cells after week 1 of culture. While this increased after a week, there was then a significant decline in CD62L expression, with limited detection possible at 4 weeks of culture. In contrast, NKG2C expression increased slightly after a week in culture, expression of NKG2C increased on the NK cells, with over 40% of the cells expressing NKG2C after 5 weeks. Thus, the culture, at 5 weeks, could be characterized as having elevated NKG2C as compared to NK cells grown without the stimulatory cytokine and having reduced or equivalent CD62L expression as compared to NK cells grown without the stimulatory cytokine. FIG. 16B shows further data supporting the development of an altered, memory-like phenotype by the NK cells. FIG. 16B shows expression data by FACS analysis of donor NK cells at day 14 (top row) and day 21 (bottom row) cultured with mbIL15-expressing cells (left column) or mbIL15-expressing cells plus IL12 and IL18 addition at day 0 (right column). CD57 expression is also shown, with the relatively low percentage of cells positive for expression confirming a trend to loss of expression of that marker when NK cells are cultured (fresh NK cells would have a higher CD57 expression). As can be seen in the mbIL15 column, NKG2C expression (X-axis) is not significantly change. In contrast (as indicated by the arrow) the percentage of cells expressing NKG2C is increased by 40% after an additional week in culture after an initial exposure to soluble IL12 and soluble IL18.

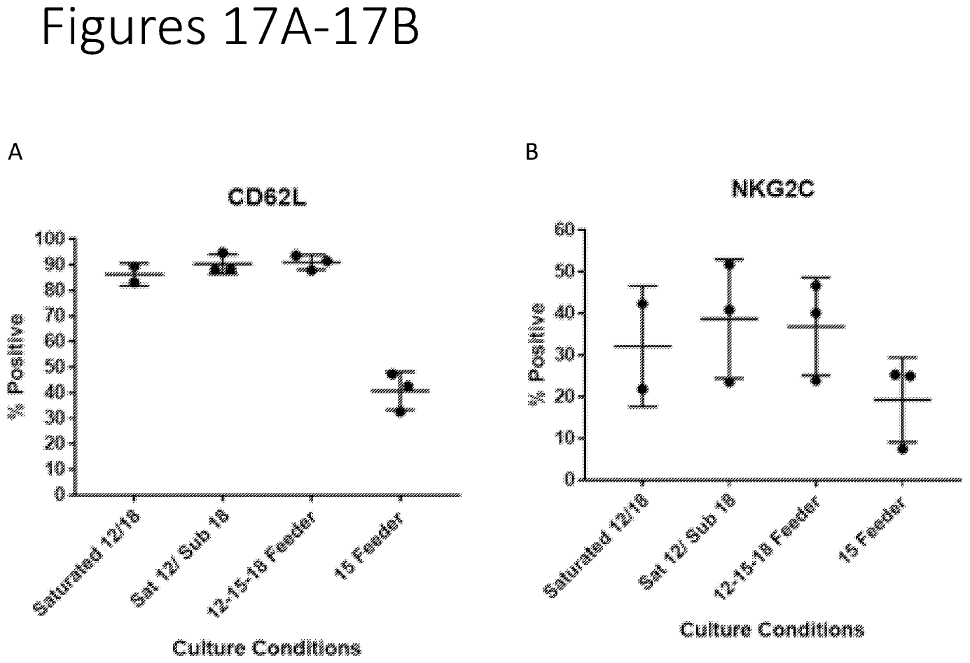

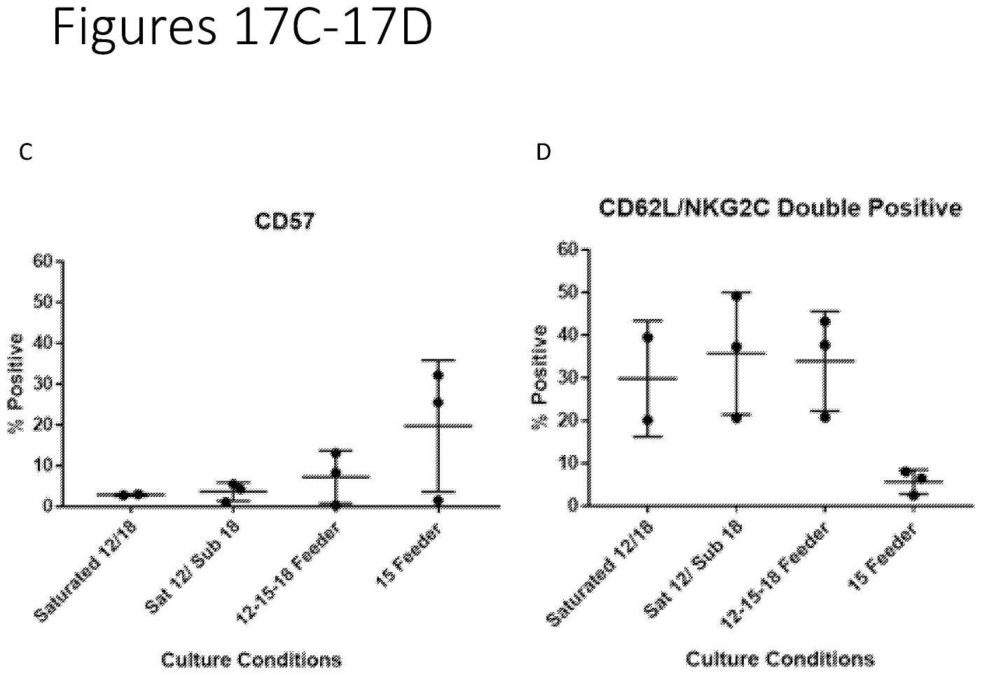

FIGS. 17A-17D show summary data related to marker expression on NK cells after 14 days in culture, under the indicated conditions. As shown in 17A, at this time point, CD62L is enhanced by the use of IL12 and IL18, whether in soluble or membrane-bound formats. As discussed above, this expression drops over additional time in culture. FIG. 17B shows enhanced NKG2D expression when IL12 and IL18 are introduced into the media at Day 1. As with other data, it is noted that the effects on the NK cell phenotype (like expansion and cytotoxicity) are roughly equivalent when the IL18 concentration is varied (e.g., effect is seen with saturated or sub-saturated concentrations of IL18). CD57 expression levels were relatively low under all conditions, reflective of the cells as cultured (rather than freshly isolated), as shown in 17D. FIG. 17D shows double positive marker expression for CD62L and NKG2C, again expression levels were enhanced with the presence of IL12 and IL18 in the culture. These data reflect the shifting phenotype of NK cells cultured with IL12 and IL18 (whether soluble or membrane-bound) towards a more potent memory-like phenotype. In several embodiments, this phenotype endows the NK cells, particularly those engineered to express a chimeric receptor, with enhanced expansion ability and/or enhanced cytotoxicity, making for a more potent cancer immunotherapy product.

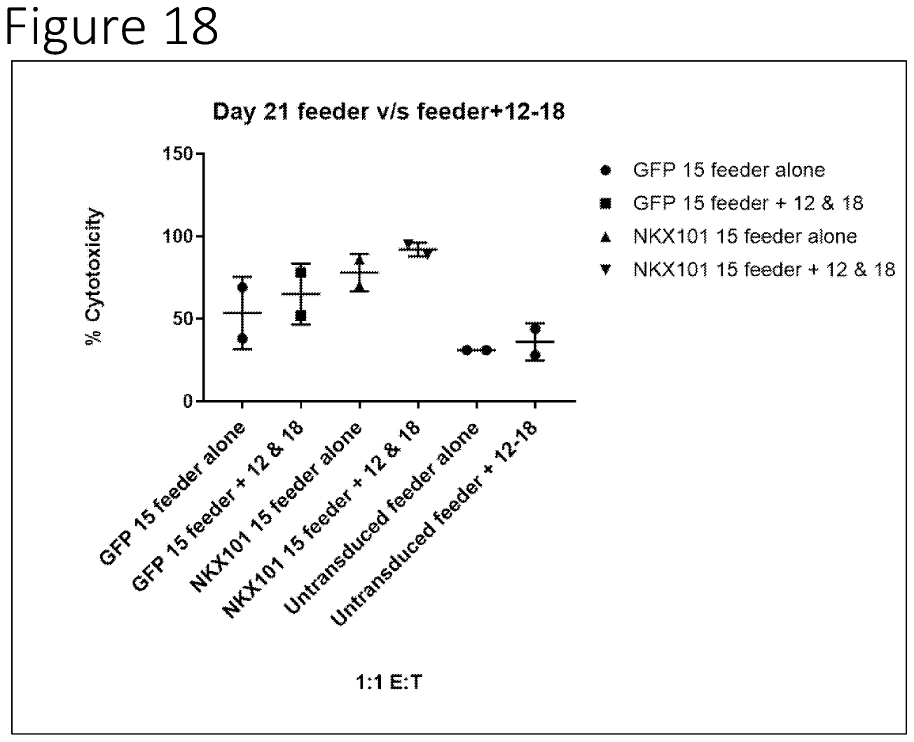

FIG. 18 shows that the use of IL12 and IL18 enhance the cytotoxicity of engineered NK cells, even at later time points (shown is cytotoxicity at 21 days post-expansion). Notably the two central points on the figure represent NKX101-transduced NK cells, which exhibit the greatest cytotoxic effect of any of the groups. Importantly, the NKX101-transduced NK cells cultured with soluble IL12 and 18 on mbIL15-expressing feeder cells show the highest degree of cytotoxicity towards target cells (by way of non-limiting example, the target here was Reh leukemia cells). Thus, according to several embodiments, the use of soluble stimulatory factors, such as IL12, IL18, IL21 and the like, in culture of NK cells, provides for an unexpectedly improved expansion of the cells (which is highly relevant for producing clinically meaningful cell numbers) as well as unexpectedly enhanced cytotoxicity against target cells.

### Example 3—Evaluation of Expansion, Cryopreservation and Cytotoxicity

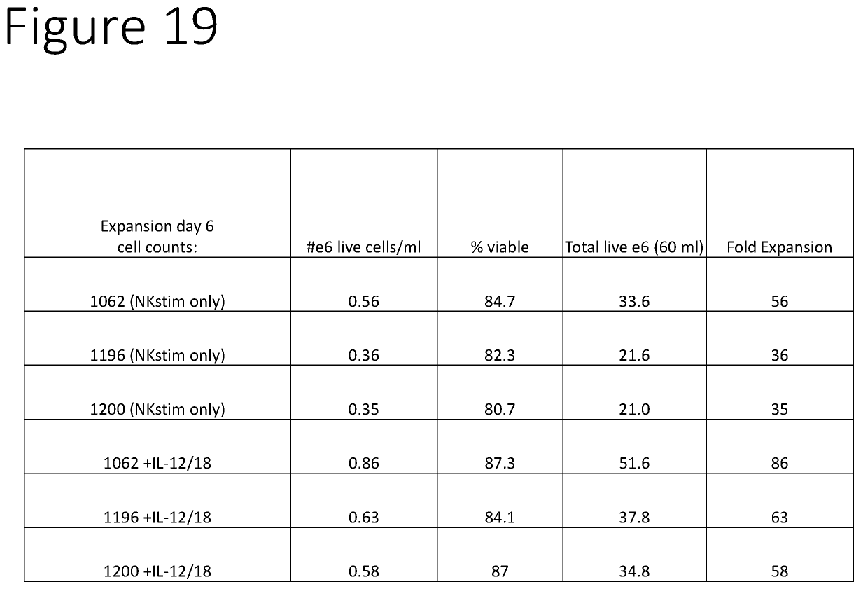

As disclosed herein, in several embodiments, the engineered NK cells that are expanded are for use in an autologous scenario. In several embodiments, an allogeneic approach is used. In several embodiments, the NK cells are designed to be “off the shelf”, referring to a pre-existing population of NK cells that has been expanded and engineered, and then is preserved for dosing to a patient at a later time. In several embodiments, the preservation is through cryopreservation. As with any freeze-thaw cycle, viability and activity of cells can be an issue. FIG. 19 shows data related to the characteristics of NK cells from three different donors cultured with mbIL15-expressing feeder cells or mbIL15-expressing feeder cells supplemented with soluble IL12/18 at the inception of culture. The bottom three rows of the table evidence the positive impacts of soluble IL12 and 18 on NK cells in culture. After day 6 of expansion, viability of NK cells in IL12/18 media was slightly higher, while the total cell number and thus, fold expansion, was notably higher when using IL12/18.

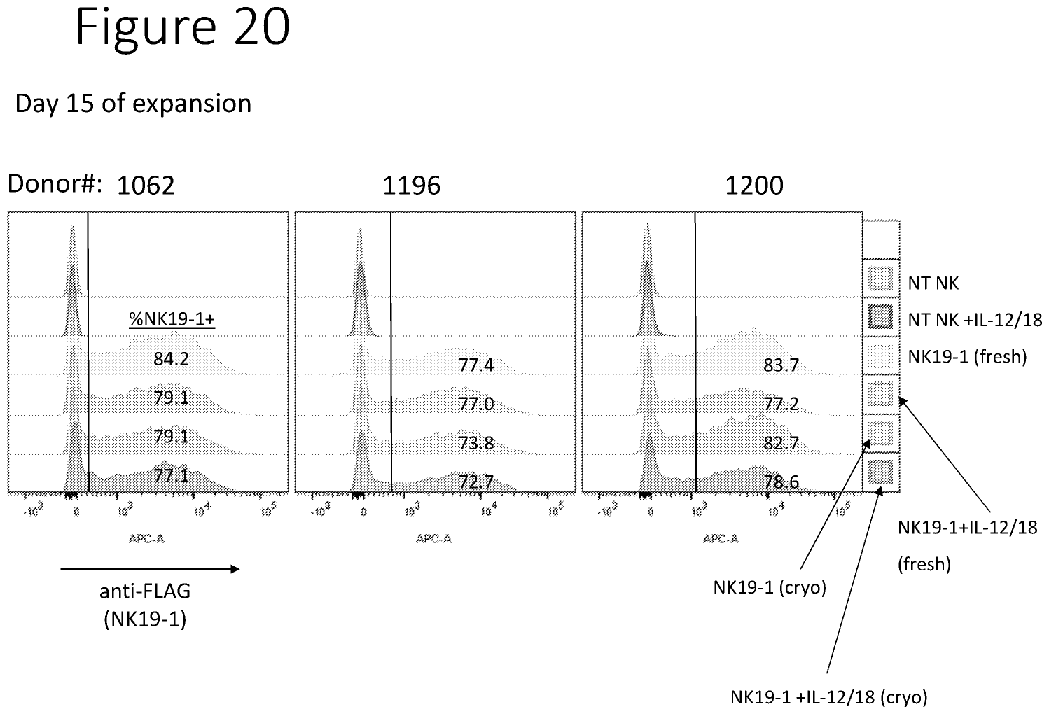

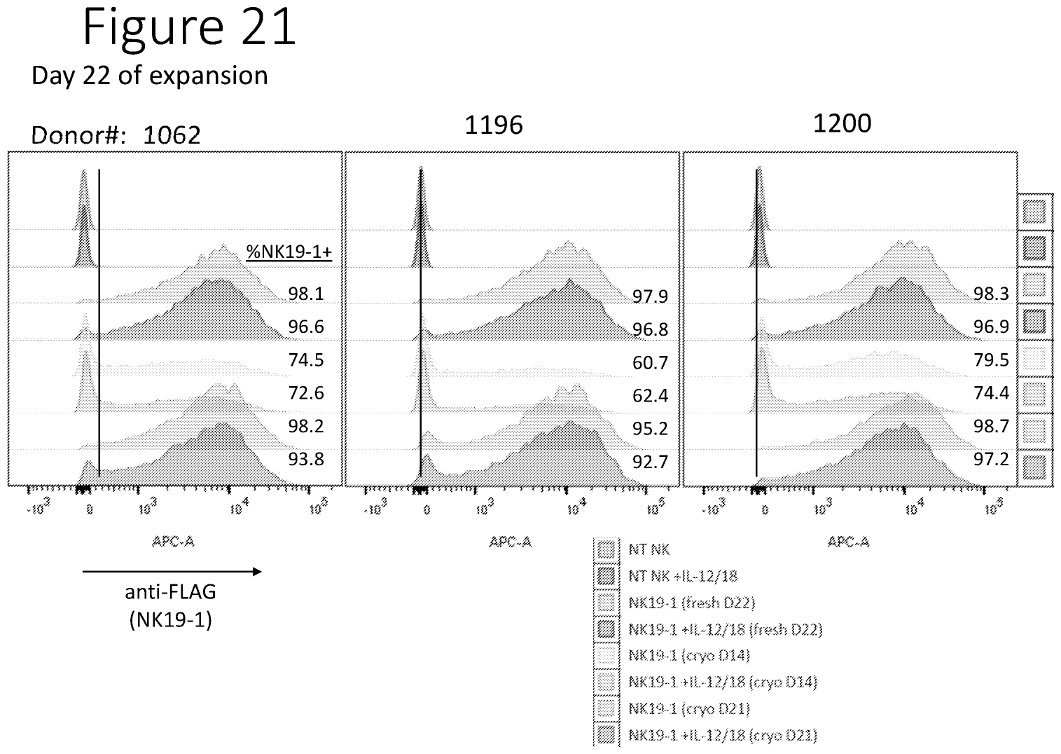

Building on this data, cells were transduced with an anti-CD19 chimeric antigen receptor and cultured with or without soluble IL12 and 18 (using mbIL15-expressing feeder cells). A portion of cells were cryopreserved and then compared with corresponding fresh cells. Using FACS, the NK cells were evaluated for expression of FLAG (the tag within the NK19-1 construct, though it shall be appreciated that corresponding non-tagged constructs are provided for herein). As shown in FIG. 20, NK cells from 3 donors both fresh and cryopreserved cells maintain expression of the CD19 CAR. The presence of IL12/18 appears to have limited impact on CAR expression. FIG. 21 shows the cells from the same donors at day 22 of expansion. Interestingly, the percentage of cells expressing the anti-CD19 CAR was reduced at day 14 as compared to day 21. The expression of the construct at Day 21, was approximately the same as in fresh NK cells (e.g., not frozen) (compare rows 3-4 with rows 7-8). These data indicate that the NK cells cultured according to methods disclosed herein are robust cell populations and able to survive cryopreservation and still maintain viability and maintain significant expression levels of cytotoxicity inducing constructs.

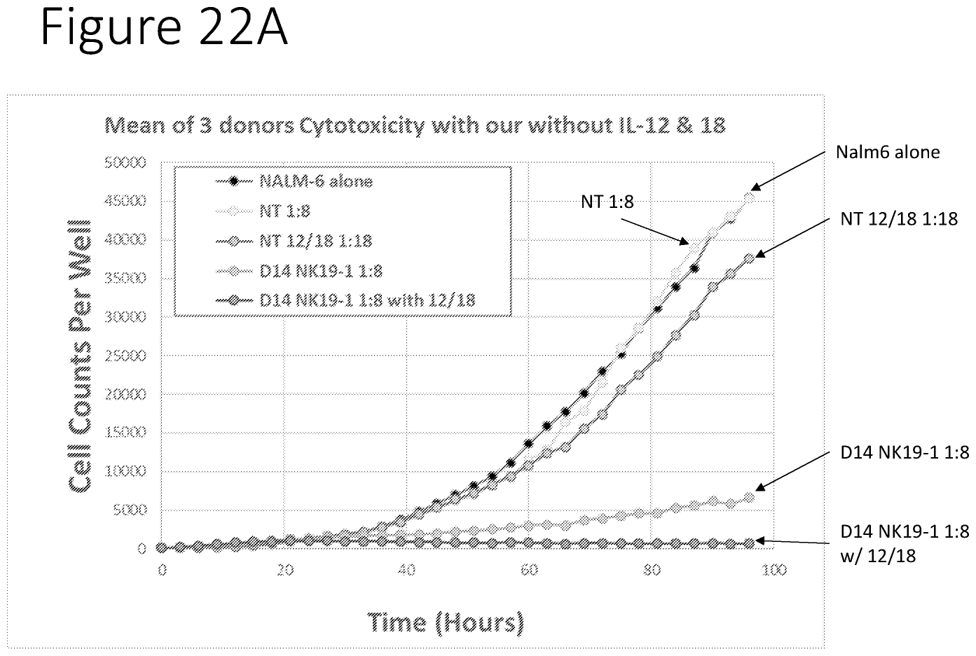

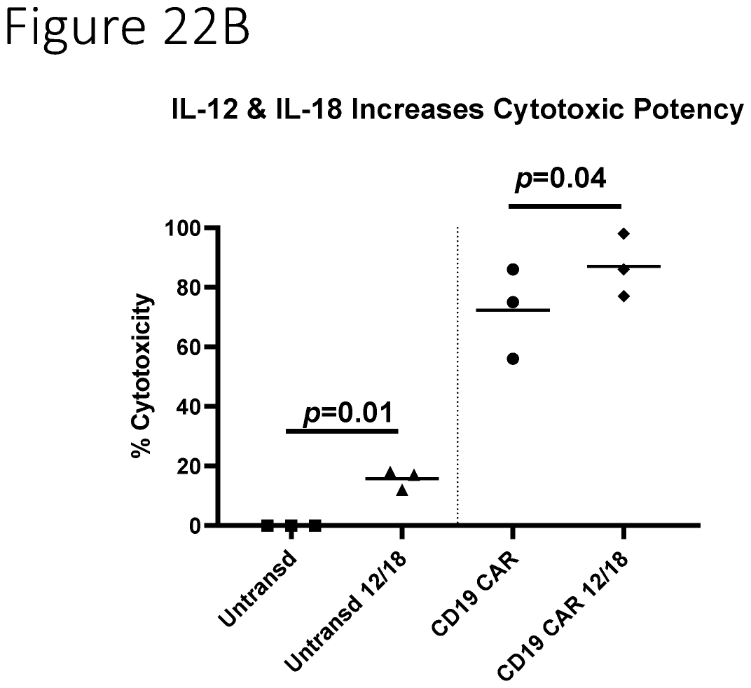

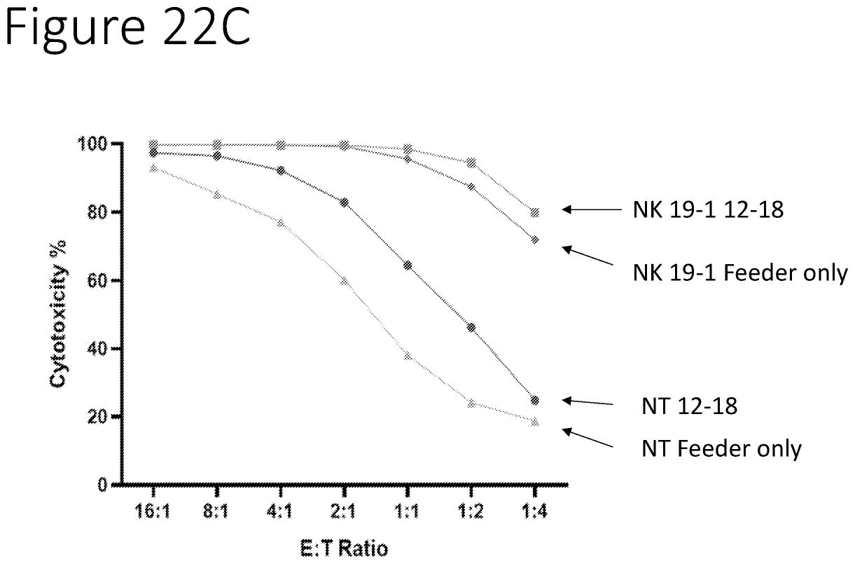

Further analysis of the effects of cryopreservation on NK cells was undertaken. A Nalm6-nuclear Red cell line was used as the target cell and were targeted by an NK cell line expressing an anti-CD19 CAR. By way of non-limiting example, this experiment employed a CAR encoded by SEQ ID NO: 1. Results of the assay are provided in FIGS. 22A-22B. FIG. 22A shows cell count curves (mean of three donors) over assay time. As shown, non-transduced NK cells and Nalm6 cells alone showing similar degrees of Nalm6 target cell increase. Non-transduced NK cells grown with soluble IL12/18 showed a slight cytotoxic effect (downward shift in the cell counts per well curve. Notably, cells that were cryopreserved at day 14 of culture showed a significant cytotoxic effect on the Nalm6 cells, limiting growth to the final hours of the experiment. Significantly, Day 14 cryopreserved cells grown in culture with soluble IL12/18 completely restricted Nalm6 cell growth. In several embodiments, cells expanded for longer periods of time (either fresh or cryopreserved) are also able to significantly reduce tumor growth. Summary data at 14 days is shown in FIG. 22B. With respect to un-transduced NK cells, expansion of the NK cells with soluble IL12/18 added at Day 0 of culture significantly increased the cytotoxicity of the NK cells against target tumor cells. Similar data are shown for NK cell expressing a CAR. Even with the presence of a CAR leading to nearly 80% cytotoxicity against target cells, culturing the CAR-expressing NK cells with soluble IL12/18 significantly enhanced the cytotoxicity. FIG. 22C shows additional cytotoxicity data for NK cells cultured in the presence or absence of IL12/18 in the culture media during expansion, at various E:T ratios. As shown cells engineered to express a non-limiting embodiment of an anti-CD19 car exhibit enhanced cytotoxicity at nearly all E:T ratios. As the number of target cells increases, the cytotoxicity of NK cells expanded using IL12 and IL18, as disclosed herein, exhibit heightened cytotoxic effects as compared to cells expanded on feeder cells alone. Collectively, these data provide evidence that the use of IL12/18 in the culture media results in enhanced proliferation of NK cells as well as enhanced cytotoxicity. Additionally, these data provide important additional evidence that the activity of the cells is preserved, even after cells are cryopreserved. This data indicates that, according to some embodiments, an “out of the freezer” engineered NK cell product with robust anti-tumor effects has been generated.

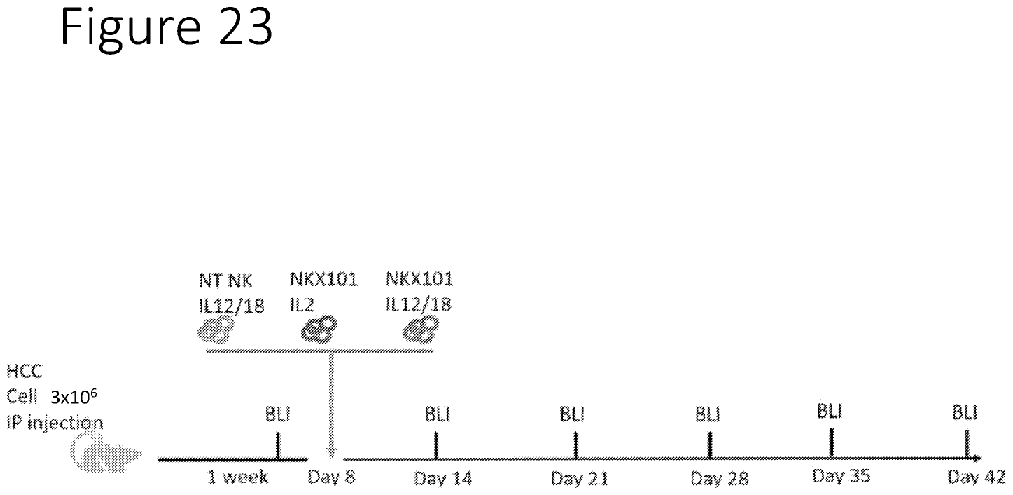

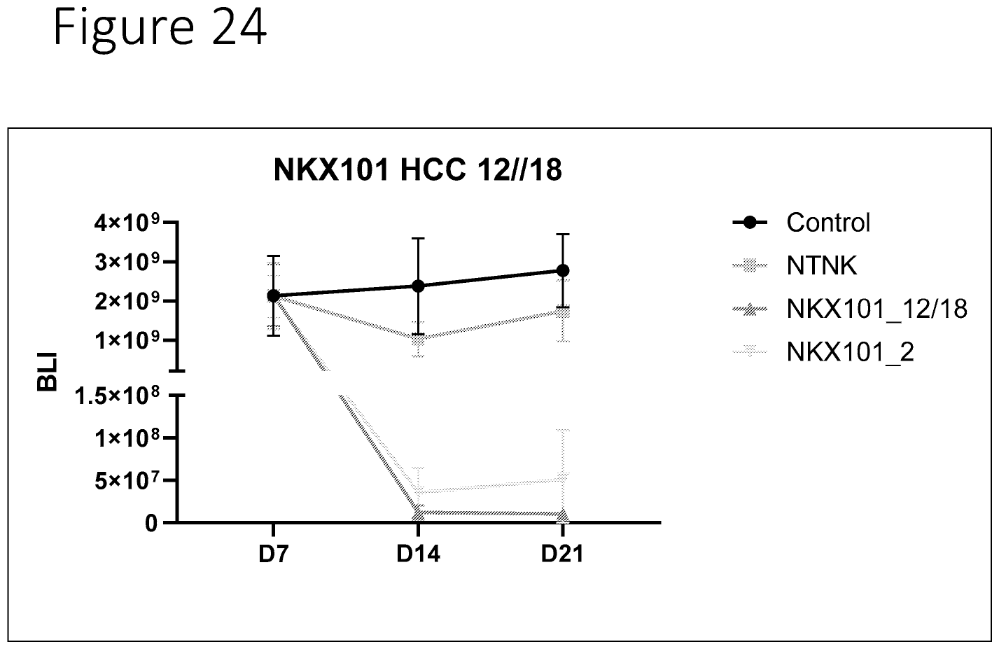

FIG. 23 shows a schematic of an in vivo experiment wherein hepatocellular carcinoma cells are injected into donor mice and NK cells grown using various culture conditions are administered. Tumor burden is thereafter monitored using bioluminescence. Administered cells are either non-transduced NK cells grown in media supplemented with soluble IL12/IL18 at day 1, NK cells expressing NKX101 grown with IL2, or NK cells expressing NKX101 grown in media supplemented with soluble IL12/IL18 at day 1. All cells were grown on mbIL15-expressing feeder cells. FIG. 24 shows the results of tumor burden analysis over time. Control animals, as well as those receiving non-transduced NK cells shown moderate tumor growth over time. In contrast, those animals receiving NK cells expressing NKX101 and grown with IL12/18 or IL2 showed significantly more anti-tumor effects. Tumor burden decreased in both group, with only a slight increase from Day 14 to 21 in the IL2 group. These data further reinforce the use of stimulatory cytokines such as IL12, IL18, or IL21 in the expansion culture media in order to enhance the cytotoxicity of the cultured NK cells.

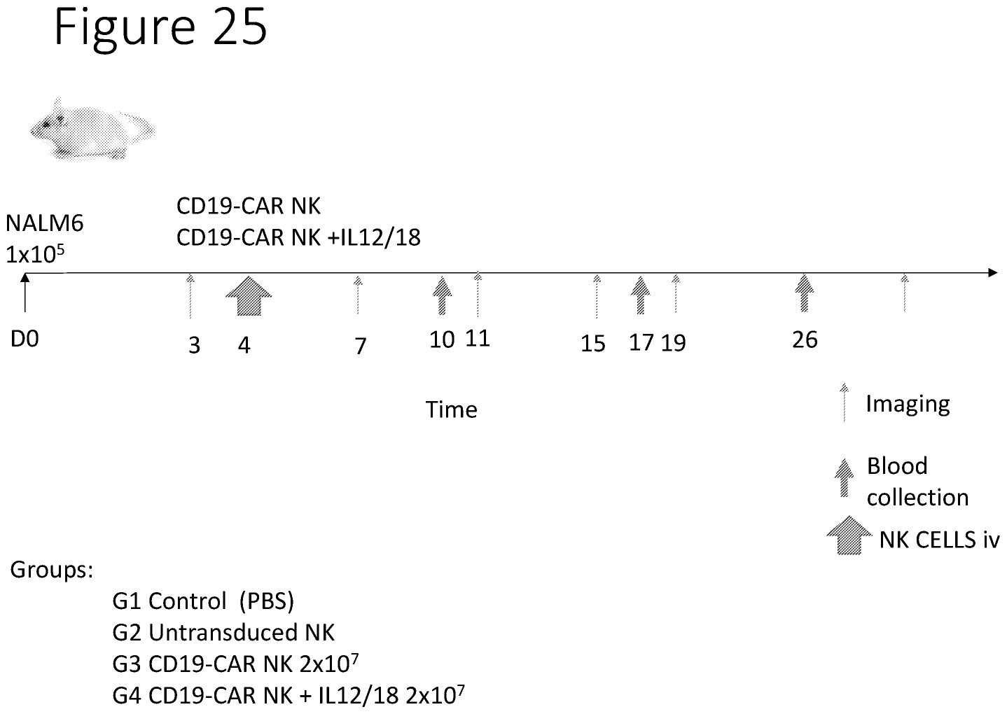

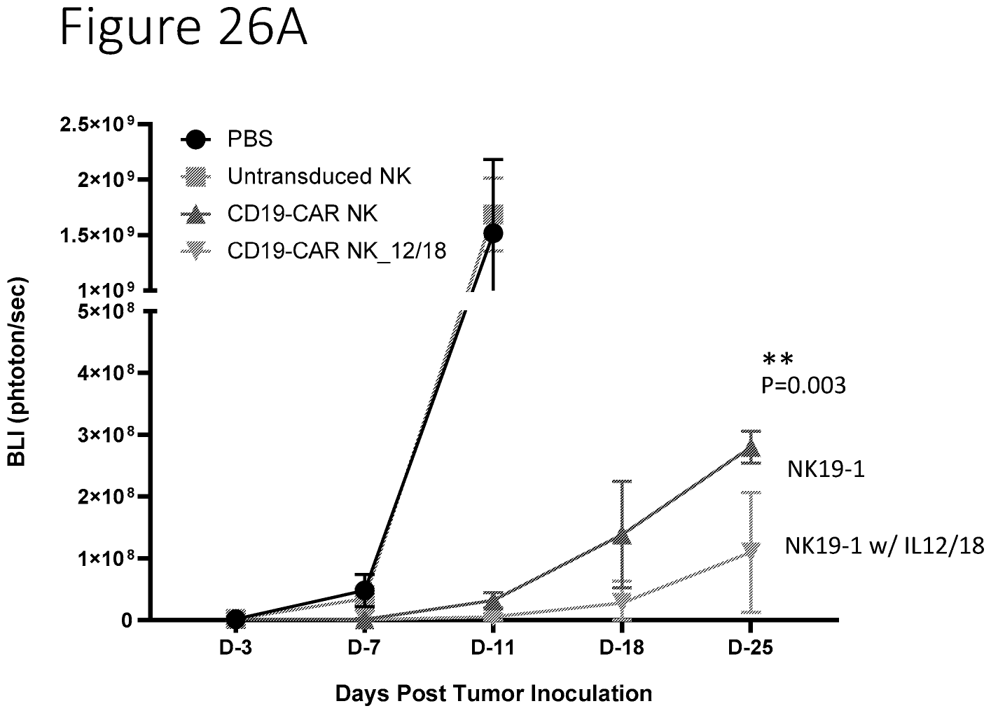

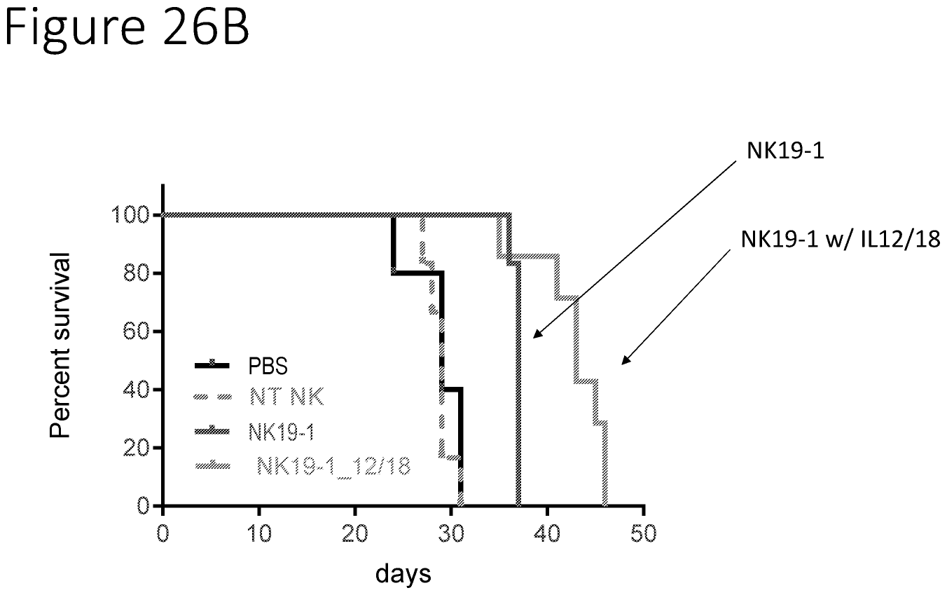

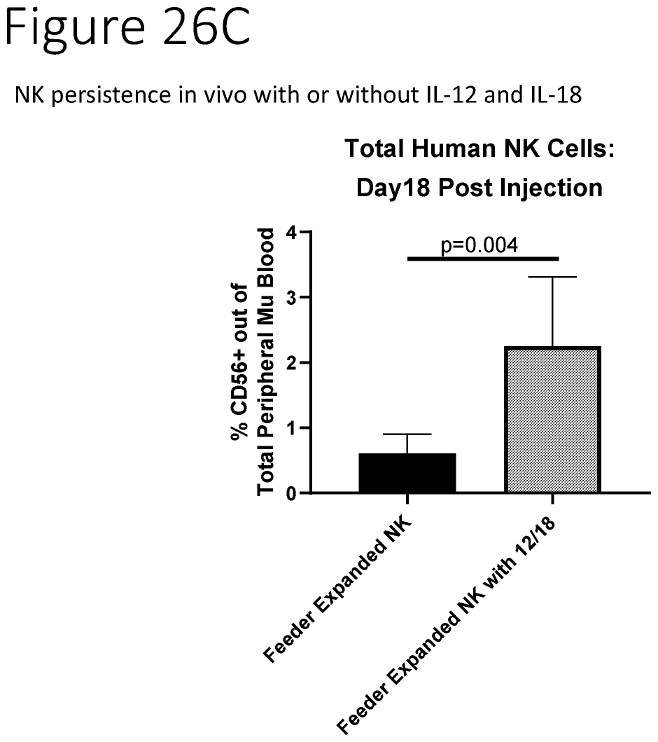

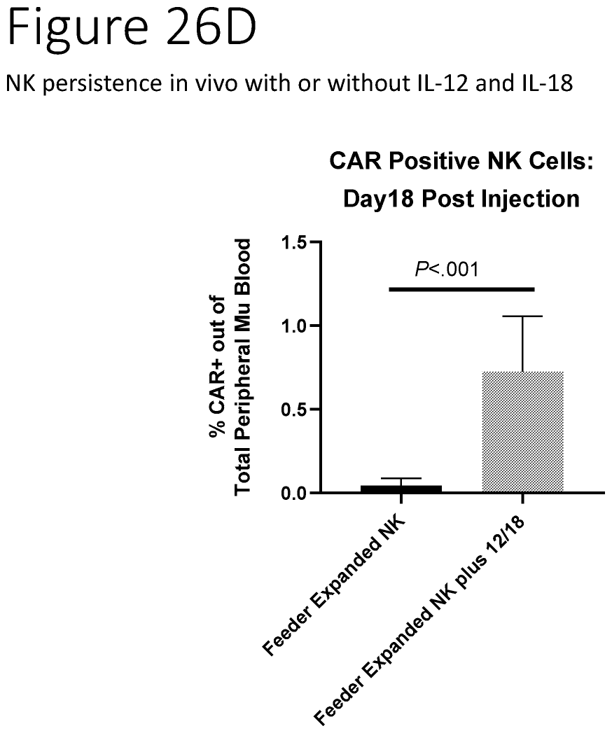

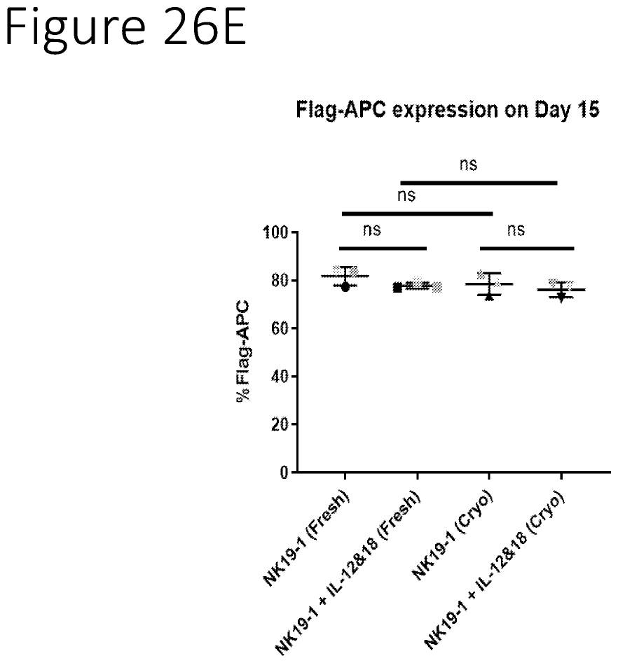

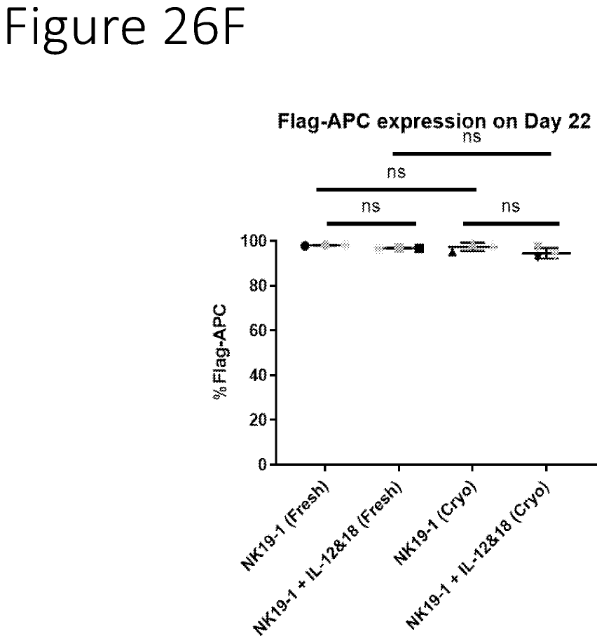

FIG. 25 shows a similar experimental setup, this time with xenograft of Nalm6 cells and treatment with NK cells expressing an anti-CD19 CAR. FIG. 26A shows the resulting bioluminescence data. As with the prior experiment, control animals and those receiving non-transduced NK cells showed a rapid increase in tumor burden, though it dropped off toward the later time points. Animals receiving NK cells expressing NK19-1 (the anti CD-19 CAR) showed an effective delay of tumor growth, limiting significant increases until the later time points. Cells expressing NK19-1 and grown with IL12/18 showed remarkable control of tumor growth, limiting increases until the late stages of the experiment and even then at markedly lower overall tumor burden as compared to other groups. Further data related to survival is shown in FIG. 26B. Mice receiving PBs (control) or NT NK cells showed a rapid drop off in survival around 30 days. NK19-1 receiving animals survived longer than those groups and NK19-1 IL12/18 animals were still 80% viable even when all other groups had no survivors. FIG. 26C and 26D show data related to the persistence of NK cells in vivo when they are cultured in media supplemented with IL12/18. FIG. 26C shows a measure of the percentage of human CD56+ cells (a marker for NK cells) out of the total peripheral murine blood. As shown, the expansion of NK cells using soluble IL12/18 results in a significantly greater percentage of human NK cells within murine blood, even at 18 days post administration. This evidences the enhanced persistence imparted to NK cells through the use of stimulatory cytokines during expansion. Likewise, it is not only NK cells generally that are persistent in vivo, but those expressing CARs enjoy enhanced persistence through the use of soluble IL12/18 (or other stimulatory molecules). FIG. 26D shows the percentage of anti-CD19 CAR positive NK cells (out of the total murine peripheral blood cell count) 18 days after injection in the xenograft recipient mice. As with the prior figure, these data show that engineered immune cells, such as NK cells expressing a chimeric antigen receptor, exhibit enhanced in vivo persistence when expanded using at least one stimulatory cytokine. An additional experiment was performed to evaluate the effects of cytokines used in expansion culture and cryopreservation (or lack thereof) on expression of CARs by NK cells. FIG. 26E shows that, at day 15 of culture, expression of a non-limiting embodiment of an anti-CD19 CAR is not changed when cytokines are used in expansion culture. That is, the enhanced effects demonstrated herein based on expansion culture using one or more additional stimulatory molecules is not counterbalanced by reduced CAR expression. Moreover, cryopreservation of NK cells does not adversely impact the expression of a CAR by the engineered NK cells. FIG. 26F confirms that CAR expression is not eroded after further time in culture. These data again support the enhanced cytotoxicity, persistence of, and stable CAR expression by NK cells grown under the influence of stimulatory cytokines, such as IL12 and IL18, among others. Likewise, cryopreservation of the engineered NK cells does not significantly adversely impact these beneficial characteristics.

### Example 4—Additional Experiments to Evaluate Effects of Cryopreservation and Expansion on Cytotoxicity, NK Cell Characteristics, and Survival of NK Cells

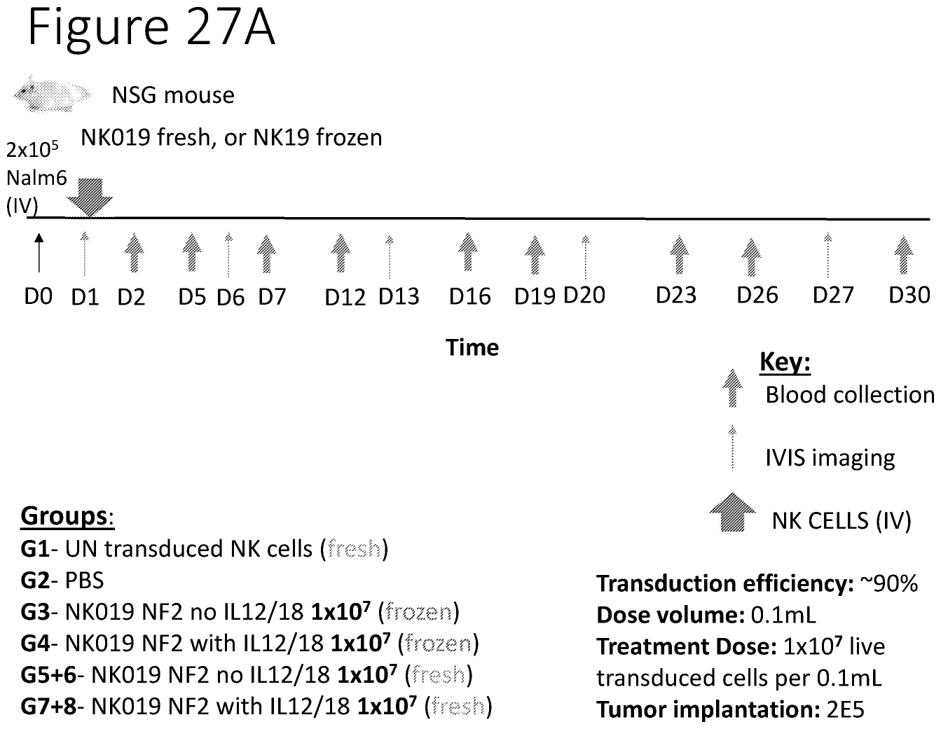

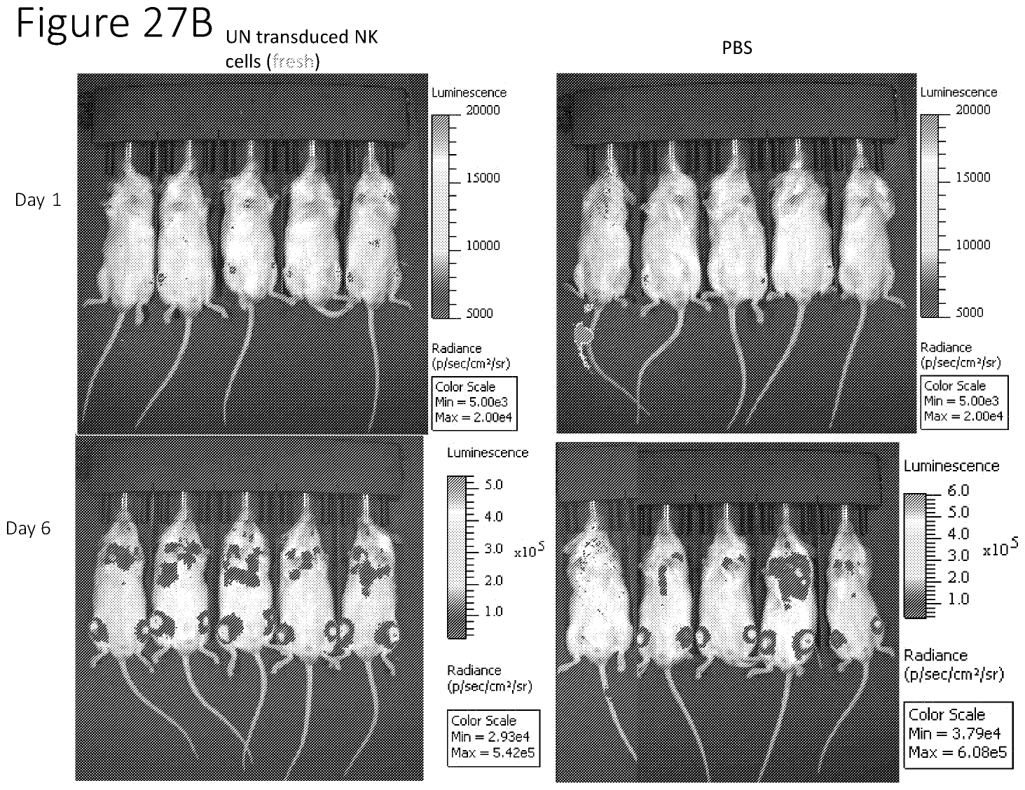

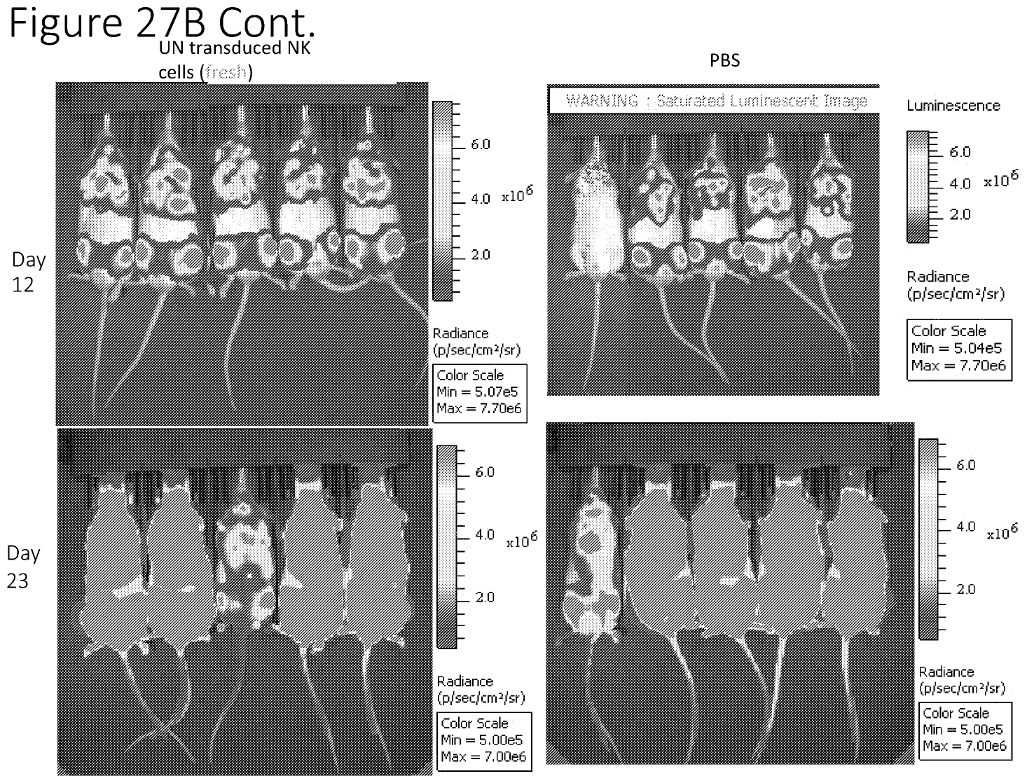

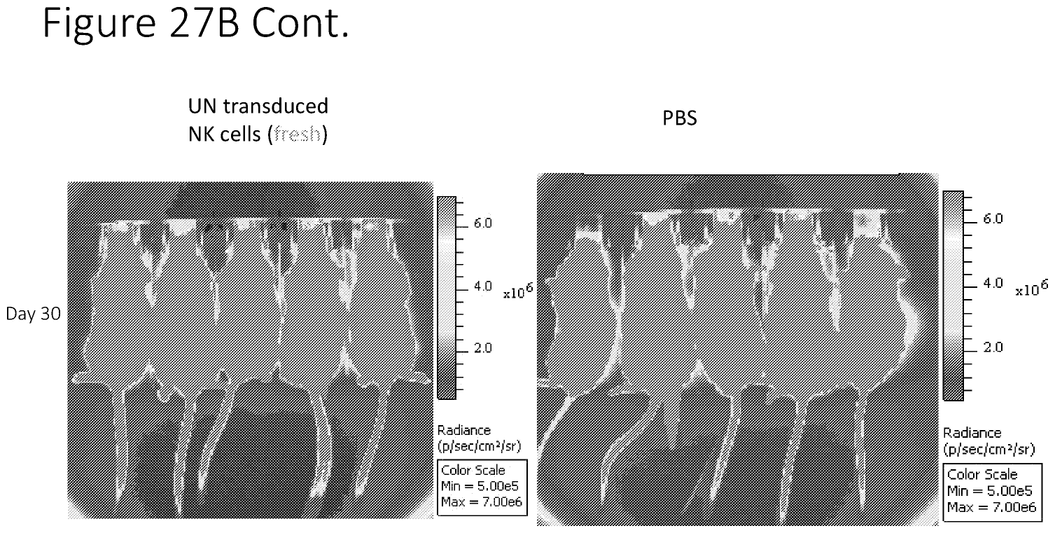

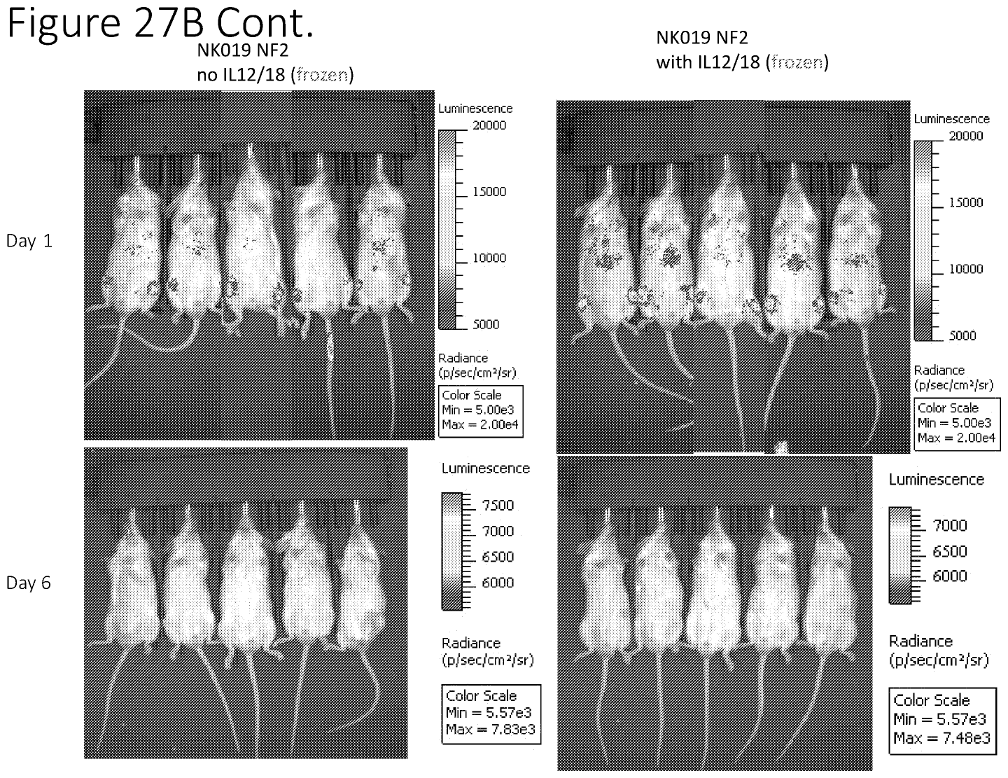

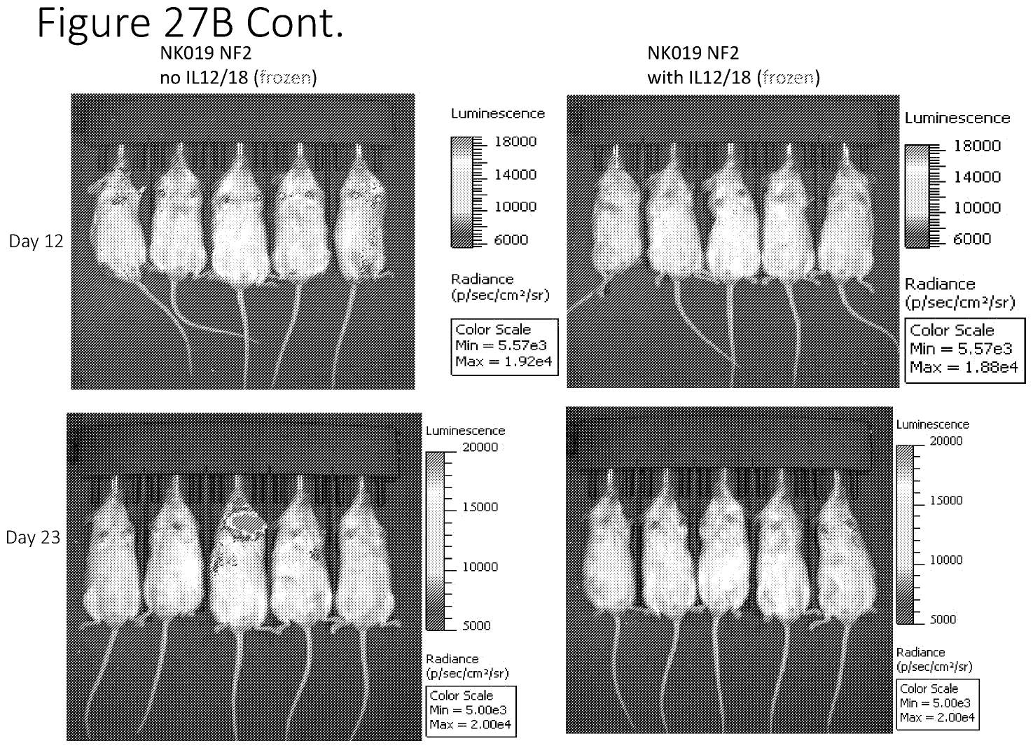

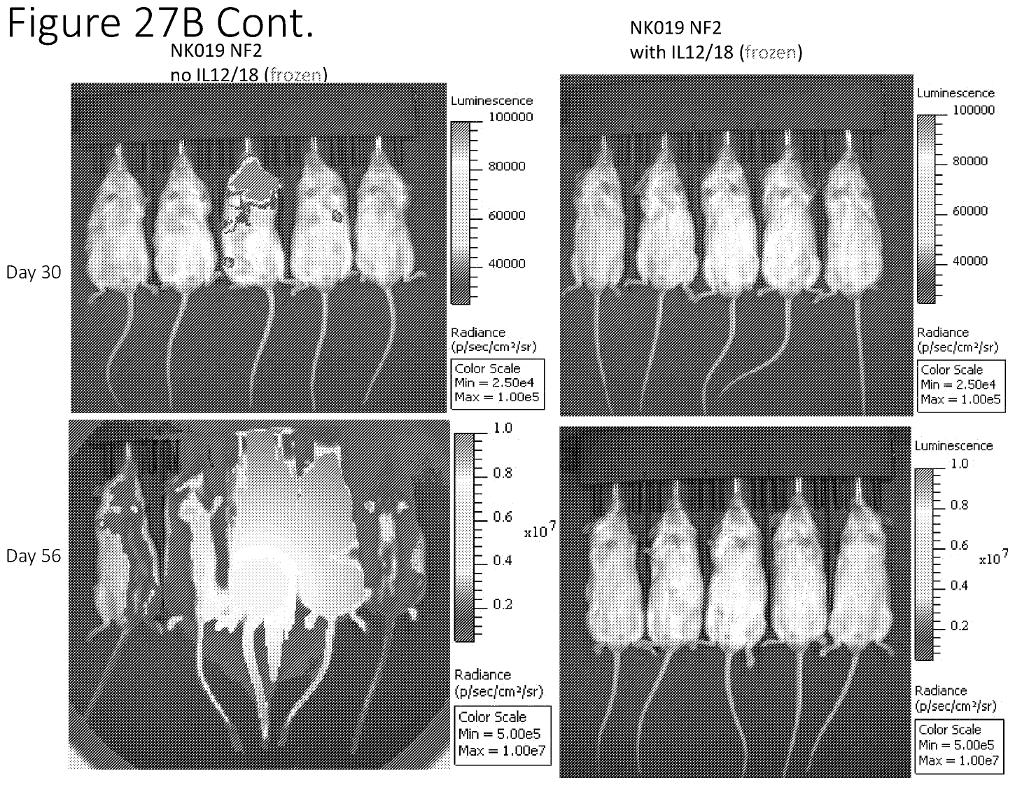

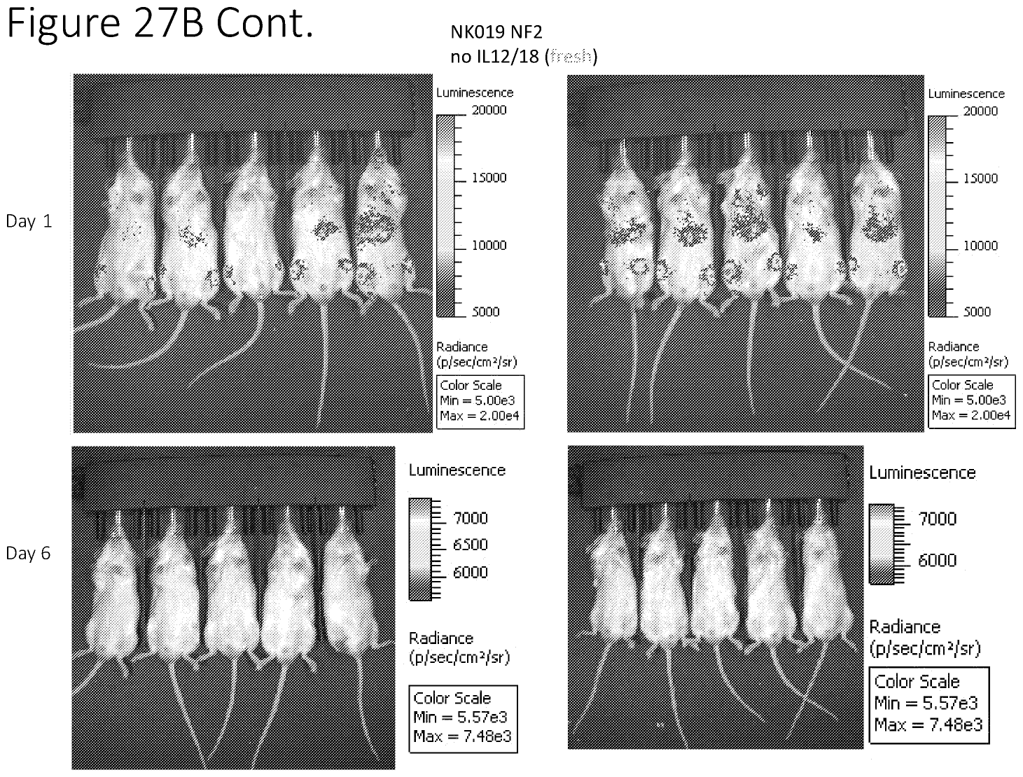

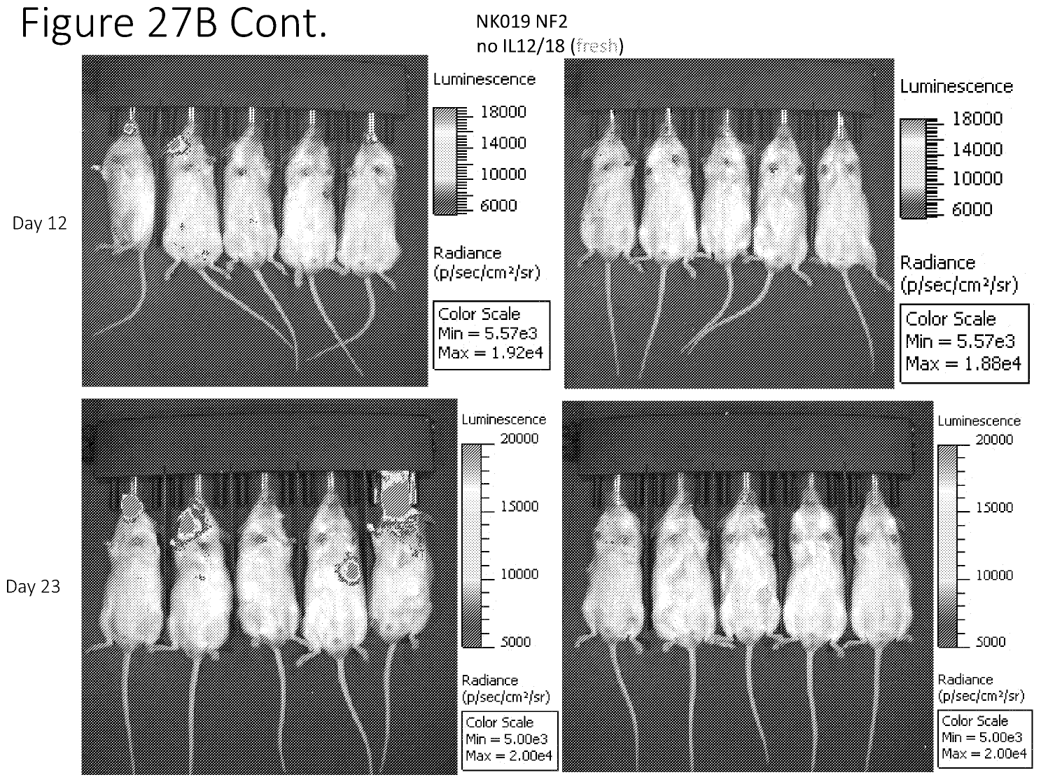

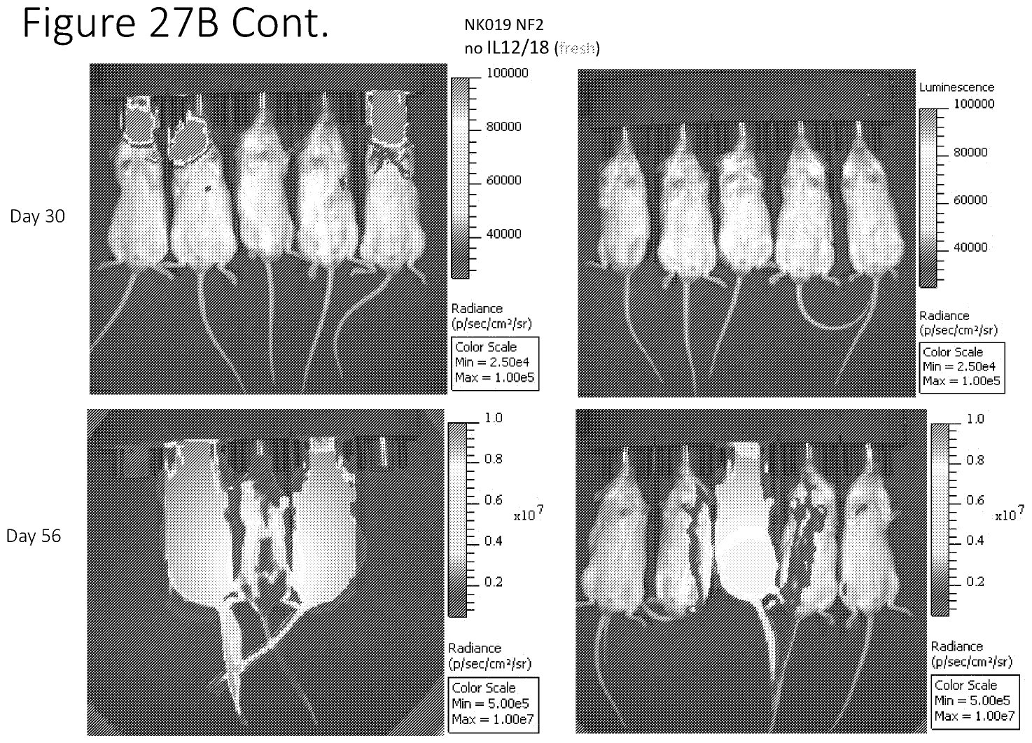

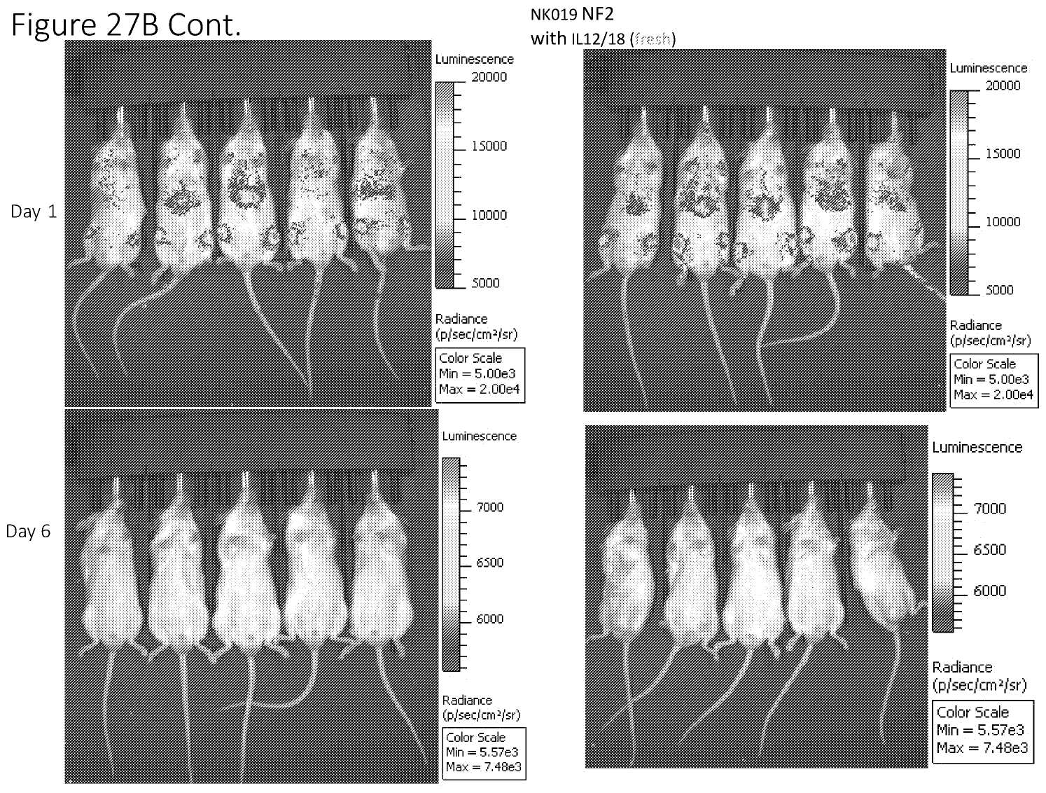

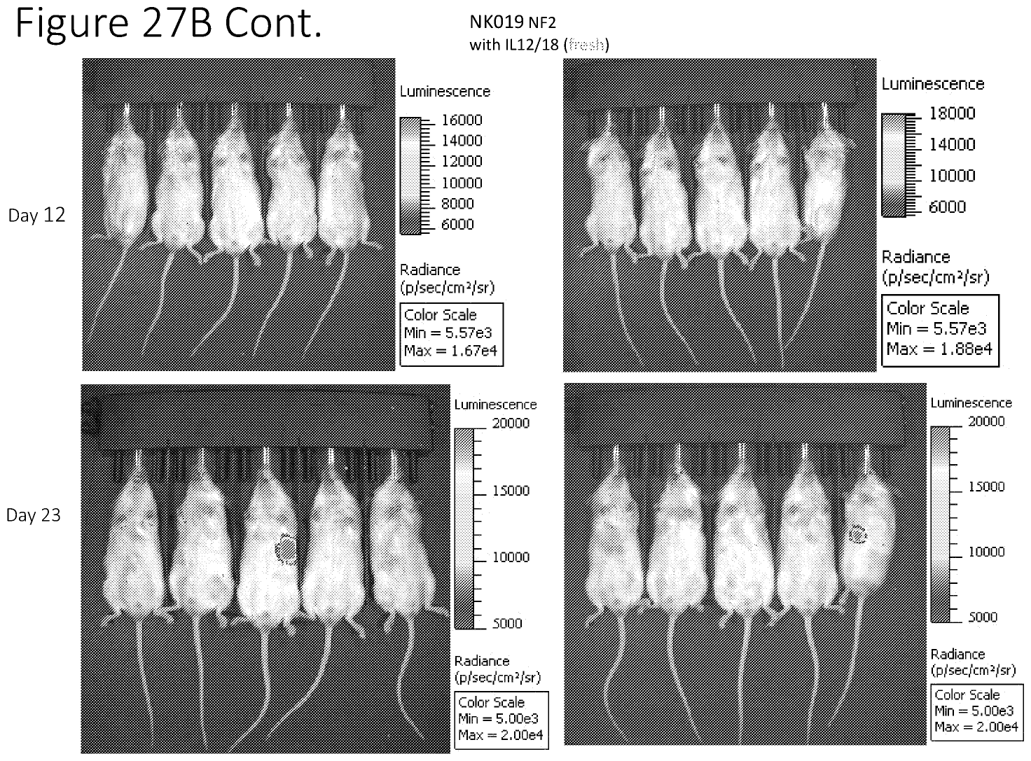

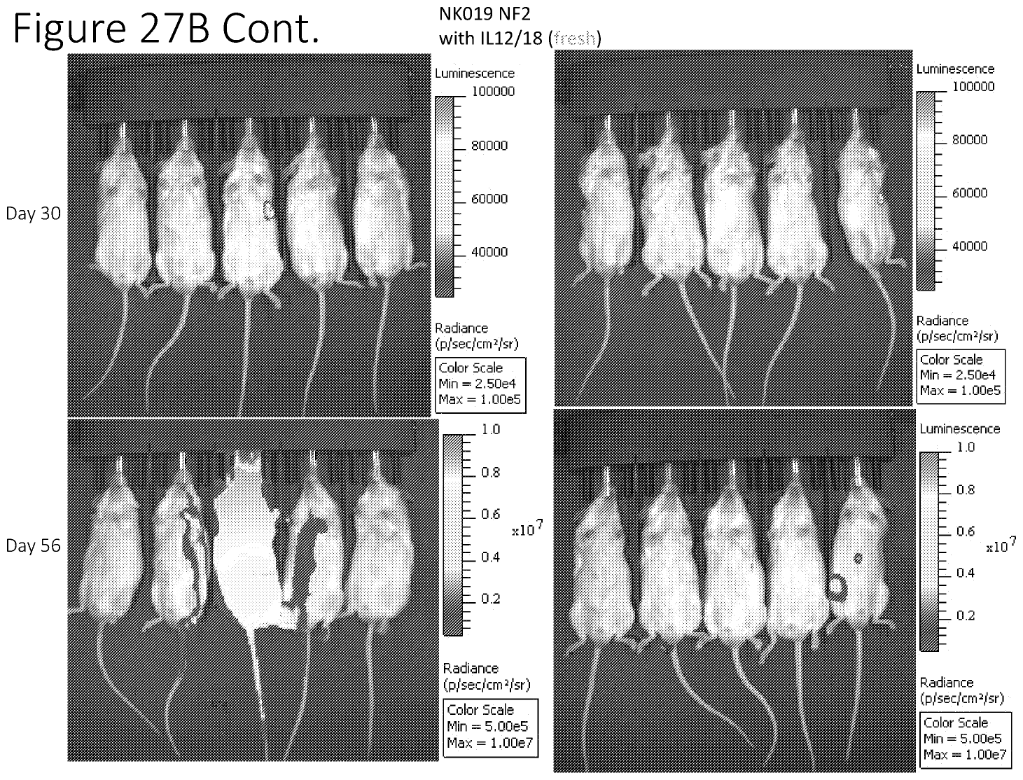

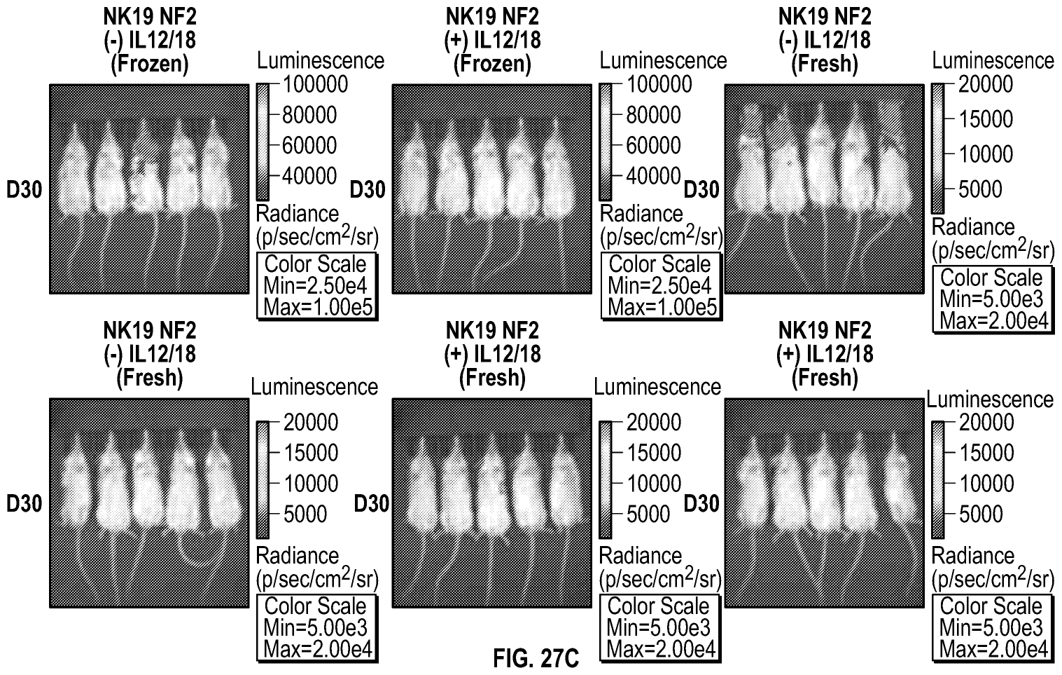

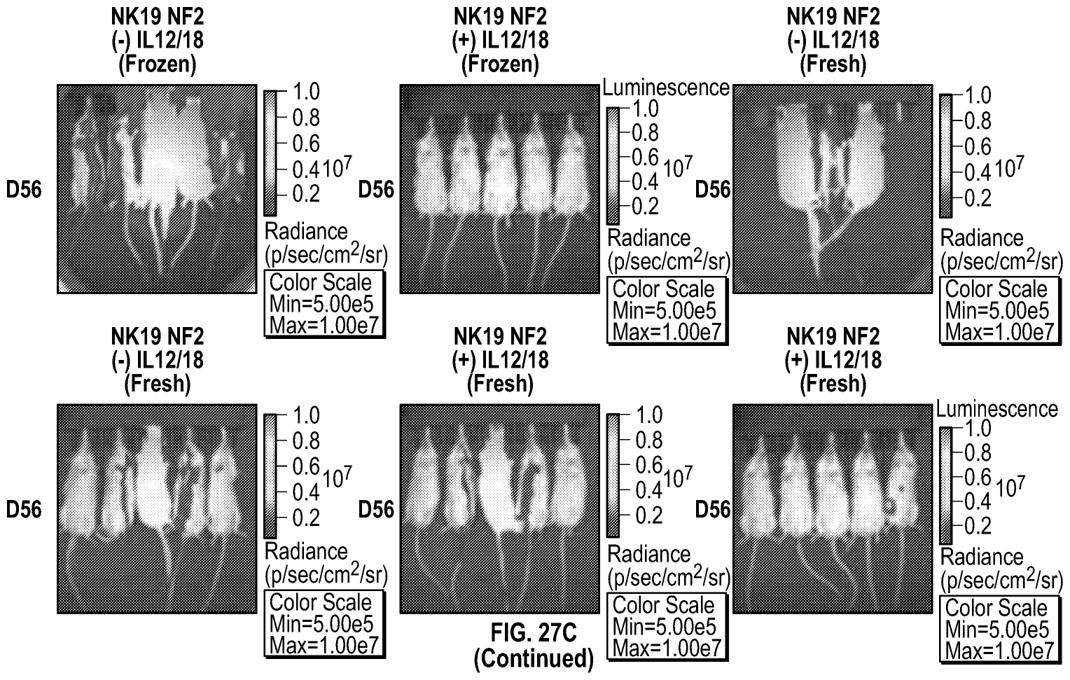

Additional experiments were performed to determine whether the process of cryopreservation followed by thawing would adversely impact the engineered NK cells, such as by reducing their viability, persistence or cytotoxicity. FIG. 27A shows a schematic experimental protocol employed, as well as the experimental groups and other conditions used. As described above, for treatment groups with an “IL12/IL18” designation, the cells were expanded in the presence of soluble IL12 and/or IL18, in accordance with embodiments described herein. Treatment groups include fresh, un-transduced NK cells (G1) and PBS (G2) as controls. Experimental groups included cryopreserved and thawed NK cells engineered to express a non-limiting embodiment of an anti-CD19 CAR and expanded without (G3) and with additional stimulatory cytokines (G4) as well as fresh NK cells engineered to express a non-limiting embodiment of an anti-CD19 CAR and expanded without (G5, G6) and with additional stimulatory cytokines (G7, G8). Blood collection and imaging were conducted at the indicated time points of FIG. 27A.

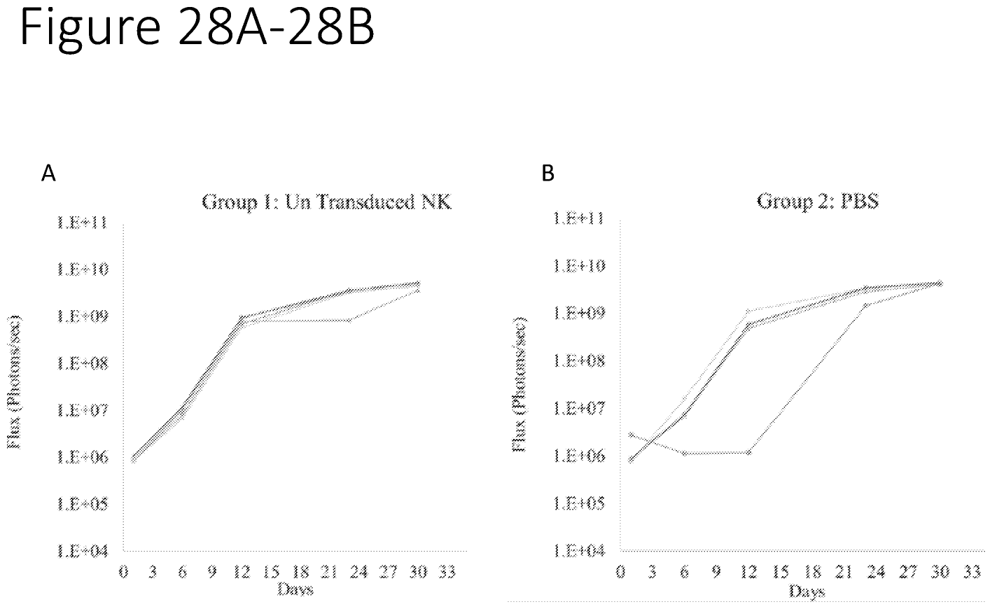

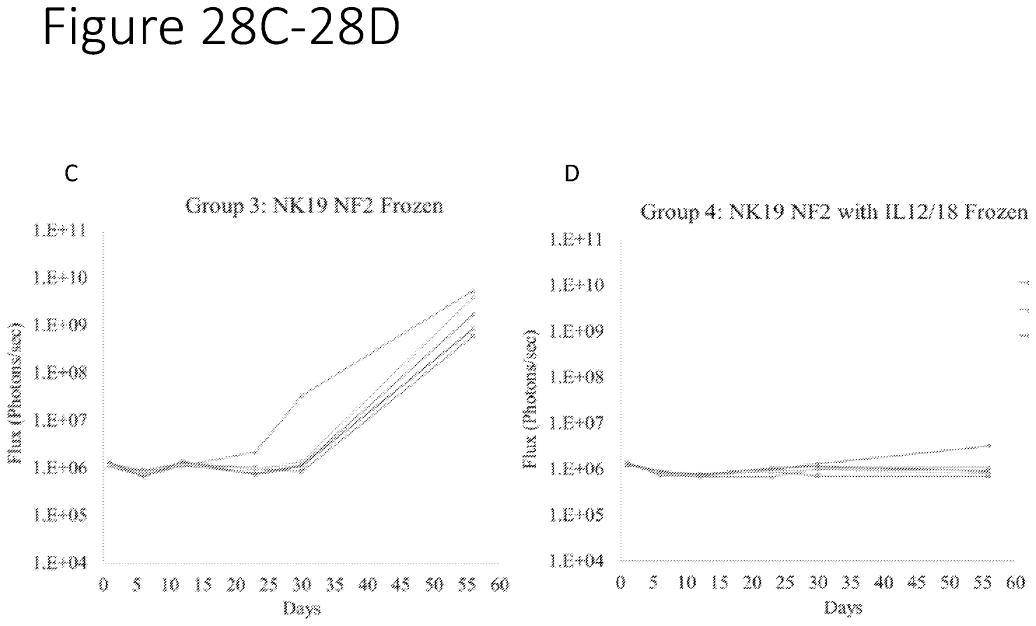

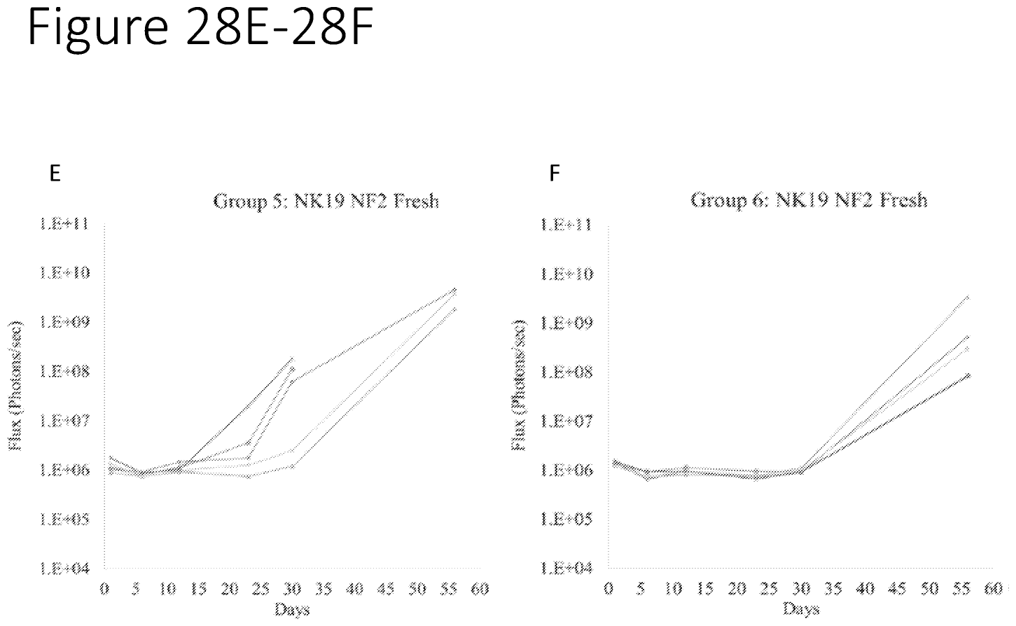

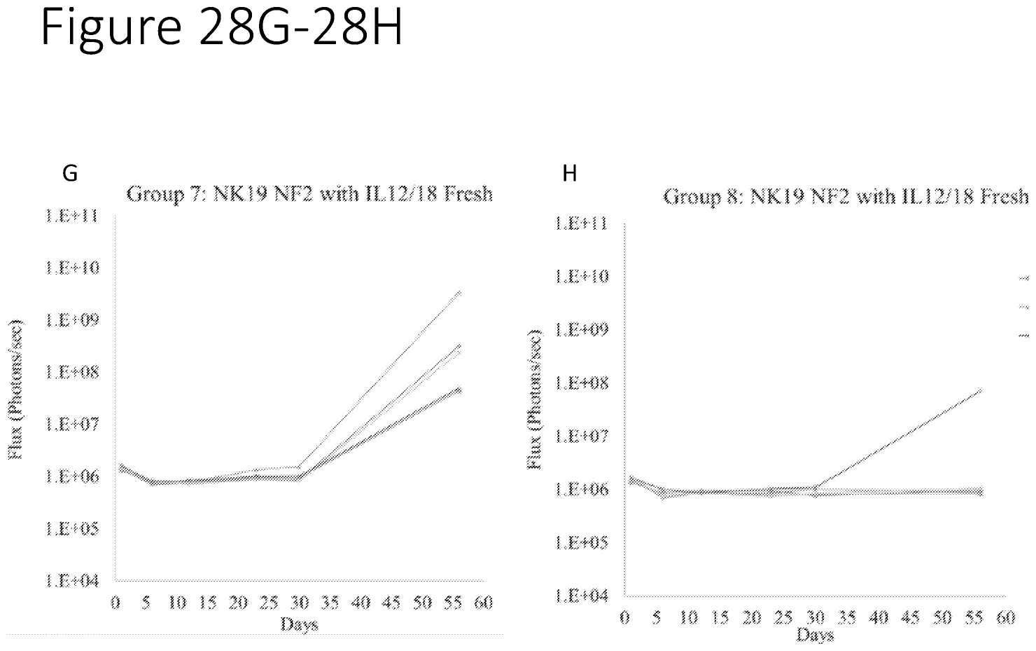

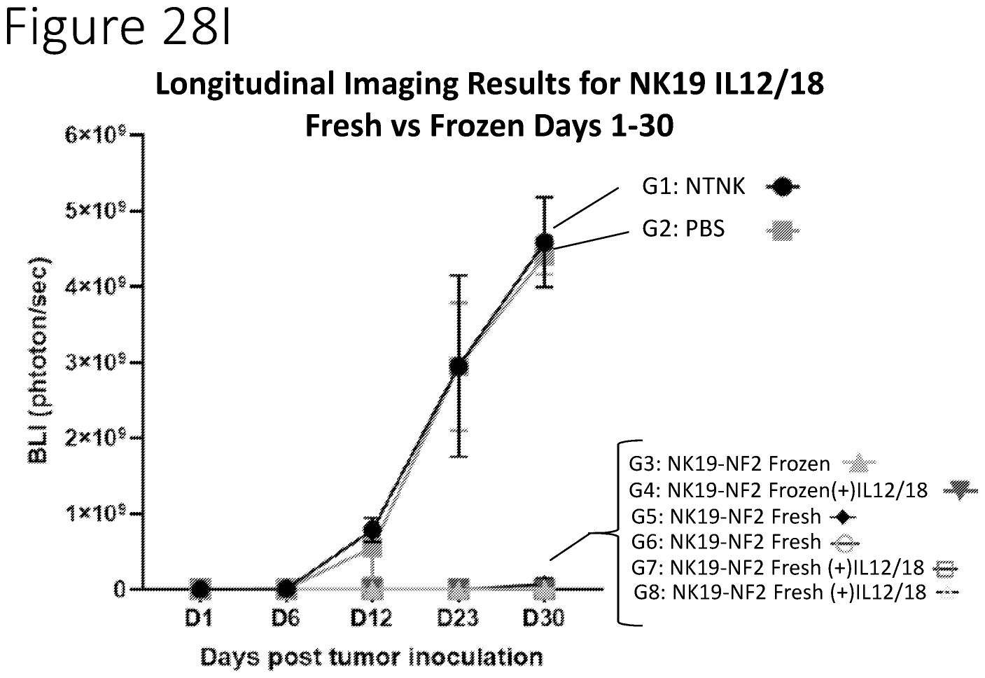

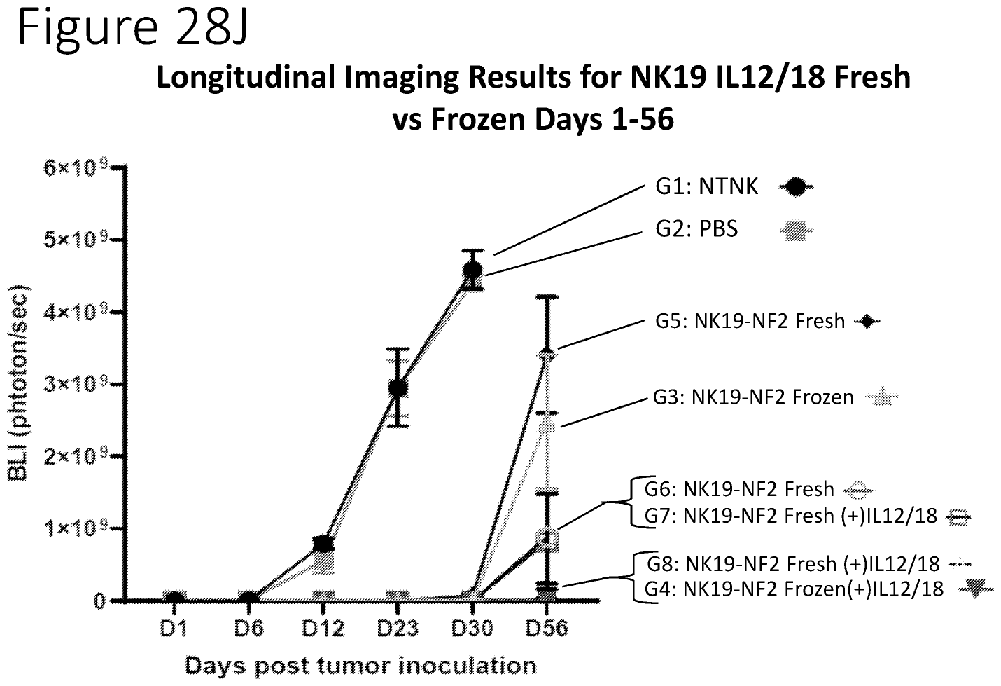

FIG. 27B and 27C shows the in vivo bioluminescence imaging from the indicated experimental groups. FIG. 28A-28H show line graphs that reflect the bioluminescence intensity over time. These data are summarized in FIG. 28I, which shows the first 30 days post-treatment, and FIG. 28J which shows data through 56 days. While FIG. 28I shows a clear distinction between the NK cells expressing CD19 CARs and the two control groups, each of the experimental groups show limited to non-detectable increases in BLI measured over the first 30 days of the experiment (increased BLI is indicative of increased tumor growth), indicative of control of tumor growth. FIG. 28J shows data through 56 days, and there is a greater separation of the experimental groups expressing the various CAR constructs and processed under the indicated conditions at inhibiting tumor cell growth. Control groups (G1 and G2) showed significantly increased tumor growth, resulting in termination of the experiment at 30 days for those groups. The group receiving fresh NK cells expressing an anti-CD19 CAR and expanded without use of soluble interleukins (G5) showed a sharp increase in BLI between days 30 and 56. Another experimental replicate of this group (G6) showed a more marked ability to inhibit tumor growth. The group receiving frozen NK cells expressing an anti-CD19 CAR and expanded without use of soluble interleukins (G3) also showed an increase in BLI between days 30 and 56, but not to the same degree as was detected with fresh cells. The experimental groups receiving anti-CD19 CAR expressing NK cells, whether fresh or frozen, that were expanded using additional stimulating factors during expansion (as according to embodiments disclosed herein) exhibited the most robust prevention of tumor growth. Notably, Groups 4 and 8, which were both cryopreserved NK cells showed the most inhibition of tumor growth. In combination with the data collected when fresh engineered NK cells were administered, these data indicate, that, according to several embodiments, engineered NK cells expressing anti-CD19 CARs are effective not only when prepared and administered fresh, but also when prepared, frozen, then thawed and administered (e.g., as in an certain allogeneic embodiments).

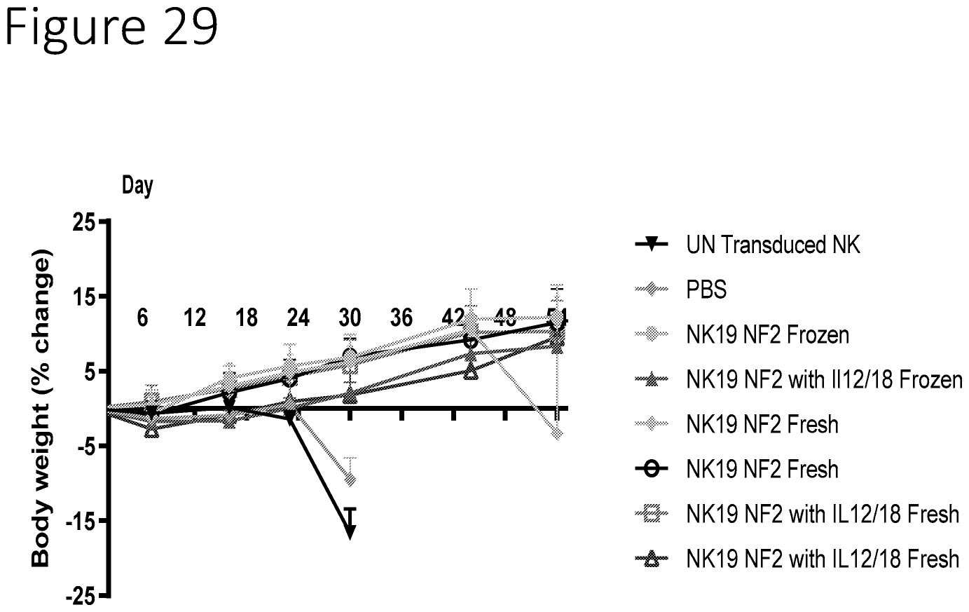

FIG. 29 shows a line graph of body mass of the mice treated with the indicated constructs over 56 days of the experiment. A reduction in body weight is correlated with increased tumor growth, e.g., progression of the tumor results in a decreased health of the mice, and corresponding loss of body weight (e.g., wasting). As shown, the control groups show substantial loss of body mass by 30 days, while all but one of the experimental groups are increasing in body mass for the majority of the experiment. As with the bioluminescence data discussed above, there is a notable trend that many of the fresh versus frozen preparations exhibit substantially similar effects on body weight. According to several embodiments, engineered NK cells expressing anti-CD19 CARs are effective not only when prepared and administered fresh. Additionally, according to several embodiments, engineered NK cells expressing anti-CD19 CARs are effective not only when prepared, frozen, then thawed and administered (e.g., as in an allogeneic context).

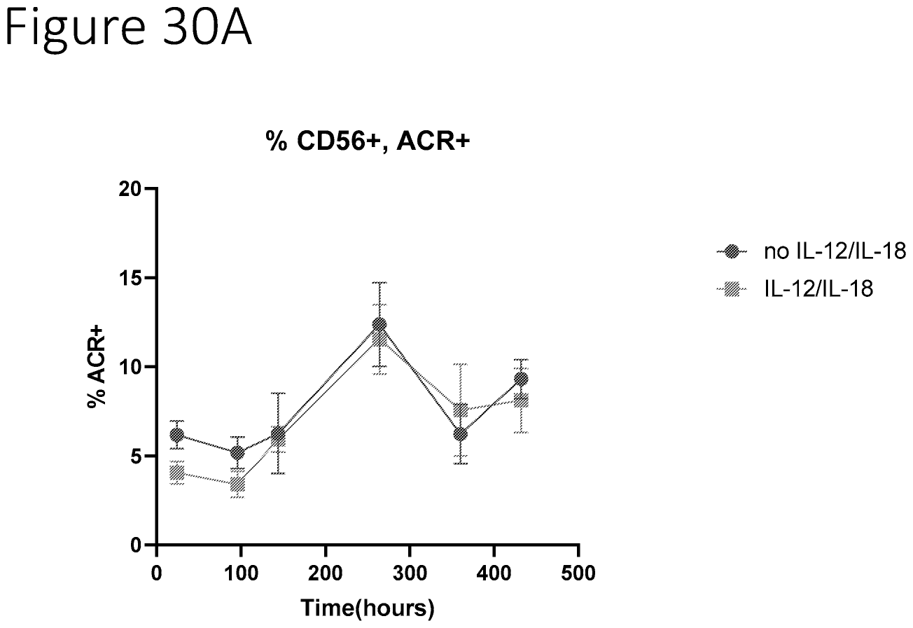

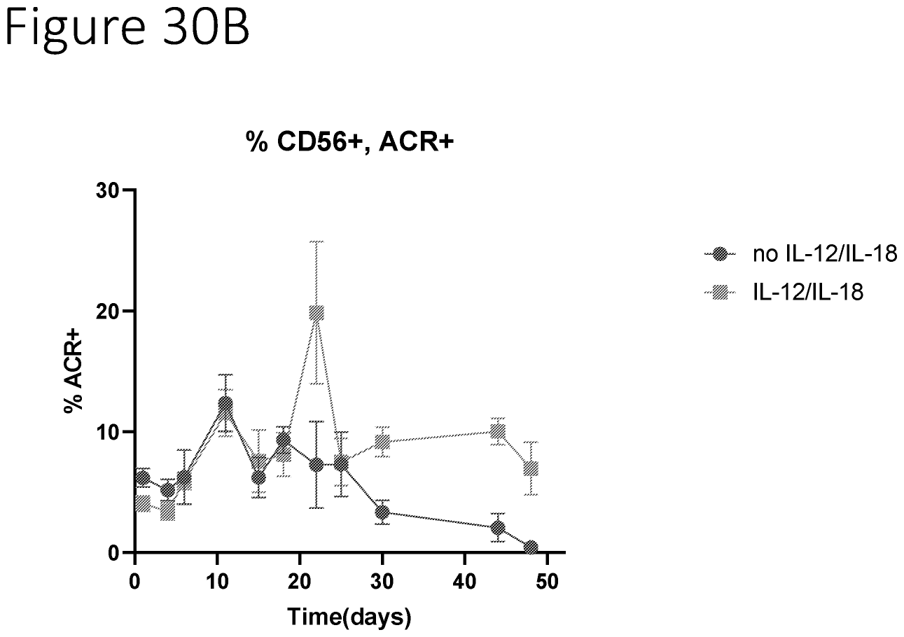

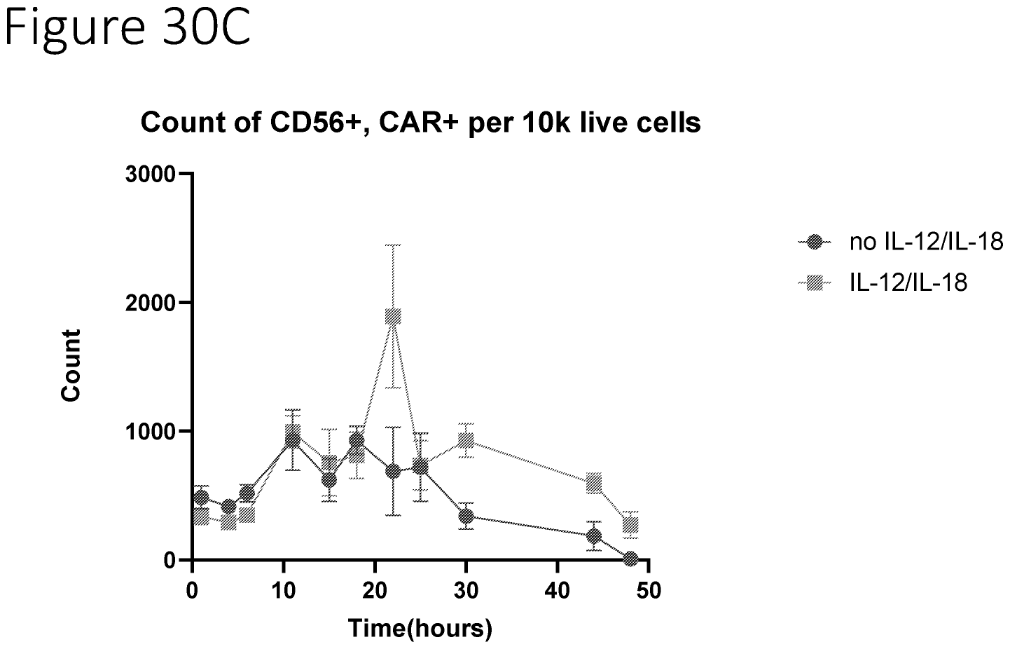

Additional data were collected to characterize the features of NK cells expanded with or without the use of one or more additional stimulatory factors. FIG. 30A shows data related to the longevity (e.g., persistence) of NK cells in culture. These data show the percentage of NK cells (based on CD56 positivity) that were engineered (based on Activating Chimeric Receptor (ACR) positivity). These data show that NK cells expanded with, or without additional stimulatory factors during expansion, such as IL12 and/or IL18, exhibit similar persistence profiles in vivo, with such engineered NK cells present at relatively consistent level in the blood (between about 5-10%) over about 7 days. Again, measuring based on detection of expression of an engineered CAR and CD56-positivity, the percentage of NK cells present in the blood of animals was measured over ˜50 days, the data for which is shown in FIG. 30B. In contrast to the similar profiles over the 7-day period, NK cells expanded without the use of one or more additional stimulatory factors began to decline in number after about 25-30 days. These cells continued a slow decline in number out to about 48 days, when cell numbers were close to zero. From the same time point of approximately 25-30 days, the engineered NK cells expanded with additional stimulatory factors (e.g., IL12 and/or IL18, according to several embodiments), continued to be present in the blood at about 10% through 45 days. Only in the last three days was there a slight decline (to about 5-7%). These data are a strong indicator that use of one or more additional stimulatory molecules, such as IL12, IL18, and/or IL21, impart engineered NK cells with an enhanced persistence in vivo, as compared to NK cells cultured/expanded without using such stimulatory molecules. FIG. 30C presents the persistence data in a different manner, based on a count of the number of engineered CAR-expressing NK cells per 10,000 live cells counted. These data mirror the general trend shown in FIG. 30B, that is, the cells expanded with the use of one or more stimulatory molecules (e.g., soluble IL12 and/or soluble IL18) remain in the blood at higher numbers over an extended period as compared to engineered NK cells expanded without such stimulatory molecules. In several embodiments, the methods disclosed herein are particularly advantageous in that they avoid cytokine addiction that is common among certain cytokine-based expansion methods. In some methods, use of high concentrations of soluble cytokines promote the growth of the cells, but the cells grow accustomed to those concentrations, and exhibit signs of withdrawal (e.g., apoptosis, reduced viability or other functional reductions) when exposed to an environment without those artificial conditions, such as upon administration to a patient. The lack of a need for ongoing high cytokine concentrations exhibited by engineered NK cells expanded according to the methods disclosed herein contributes, at least in part, to the longer life span (and active life span) of the cells in vivo.

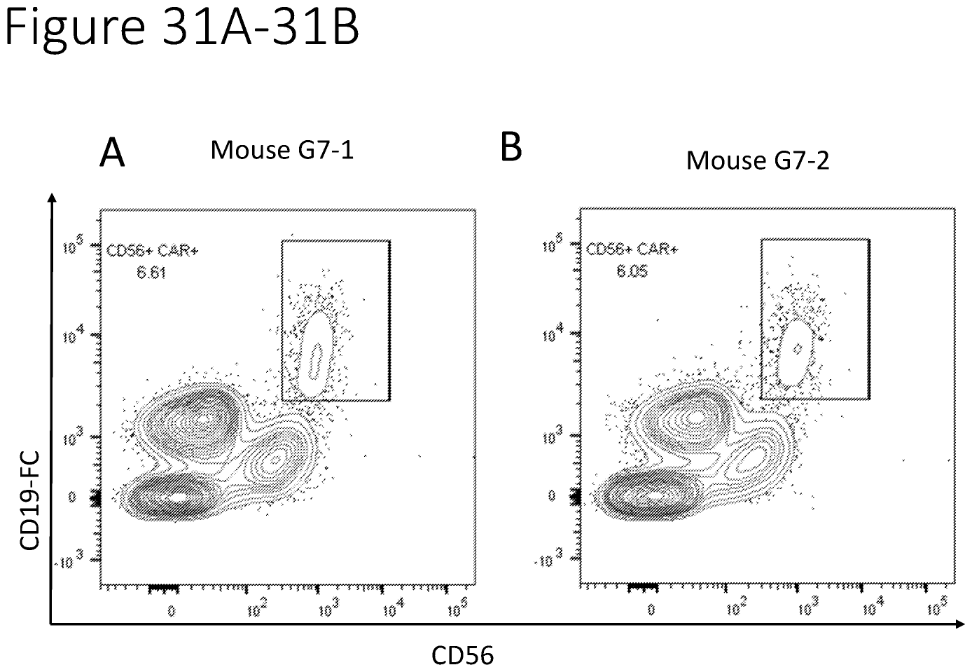

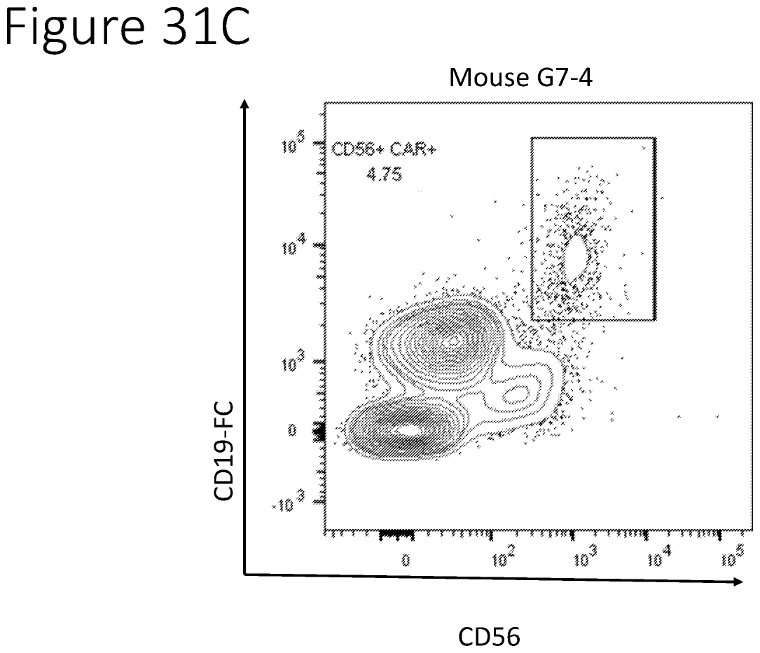

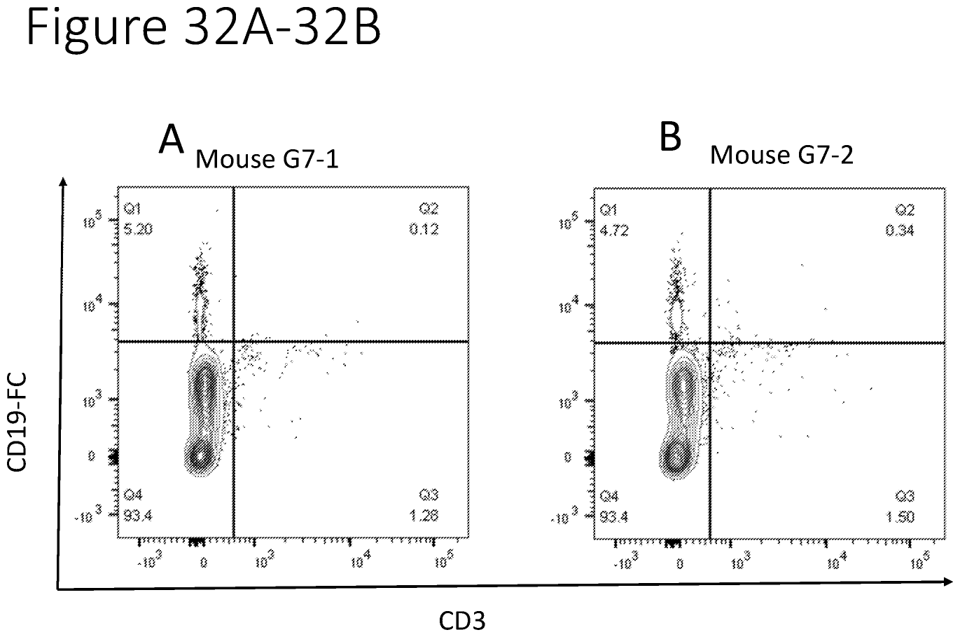

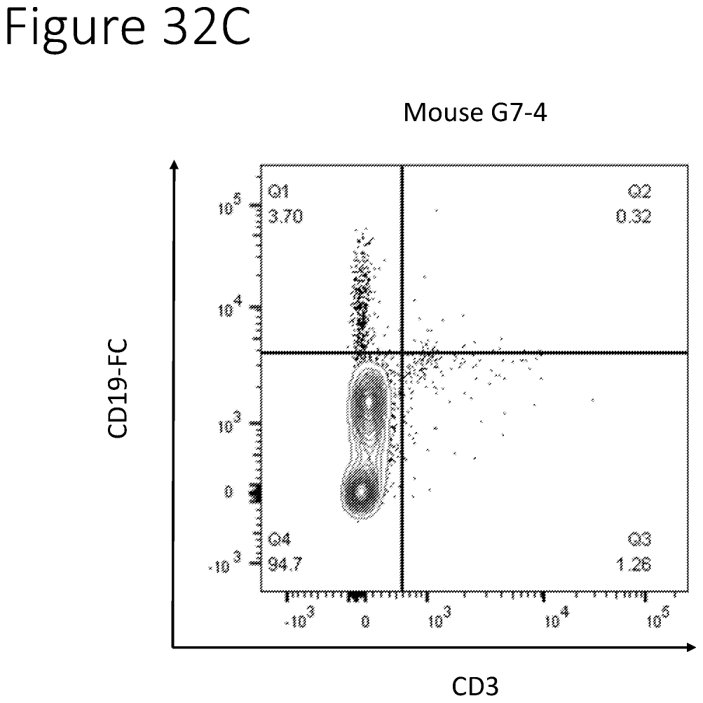

FIGS. 31A-31C shows additional data characterizing engineered NK cells produced according to embodiments disclosed herein. These data are collected from the blood of three mice (day 51 post-administration) administered fresh (not cryopreserved) engineered NK cells expressing an anti-CD19 CAR and expanded using, according to several embodiments disclosed herein, soluble IL12 and soluble IL18. The data depict the proportion of cells from a whole blood sample that are CD56-positive (indicative of NK cells) and CD19-Fc positive (indicative of cells expressing the engineered anti-CD19 CAR). As shown in each of FIGS. 31A, 31B, and 31C, the proportion of double-positive cells (boxed region in upper right) ranges from about 4.75% to about 6.7%. FIGS. 32A-32C show analysis of whole blood from the same mice as in FIG. 31, but identify cells that are CD19-Fc positive (indicative of cells expressing the engineered anti-CD19 CAR) and CD3-positive (indicative of T cells). These data demonstrate that the vast majority of cells expressing the anti-CD19 CAR are negative for CD3, which means that they are not T cells. According to several embodiments, certain NK cell production methods do involve steps to remove T cells from an initial donor whole blood sample, however, a nominal number of T cells may remain. In several embodiments, however, in accordance with the data shown in FIGS. 31A-32C, the majority of engineered cells expressing an anti-CD19 CAR exhibit features of NK cells (CD56- positive) and no features of T cell (CD3-negative).

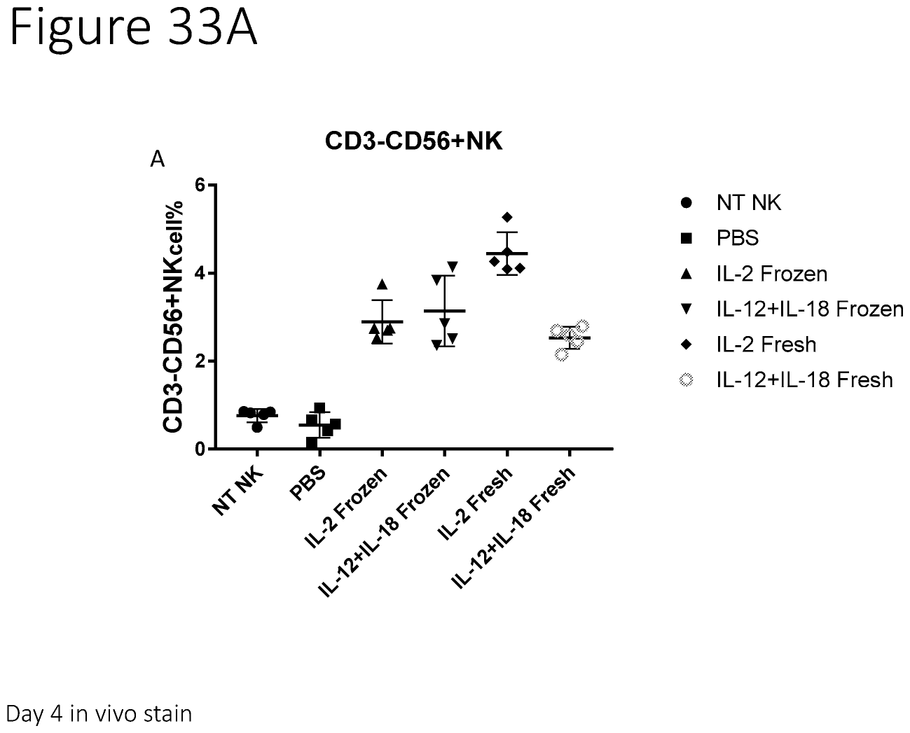

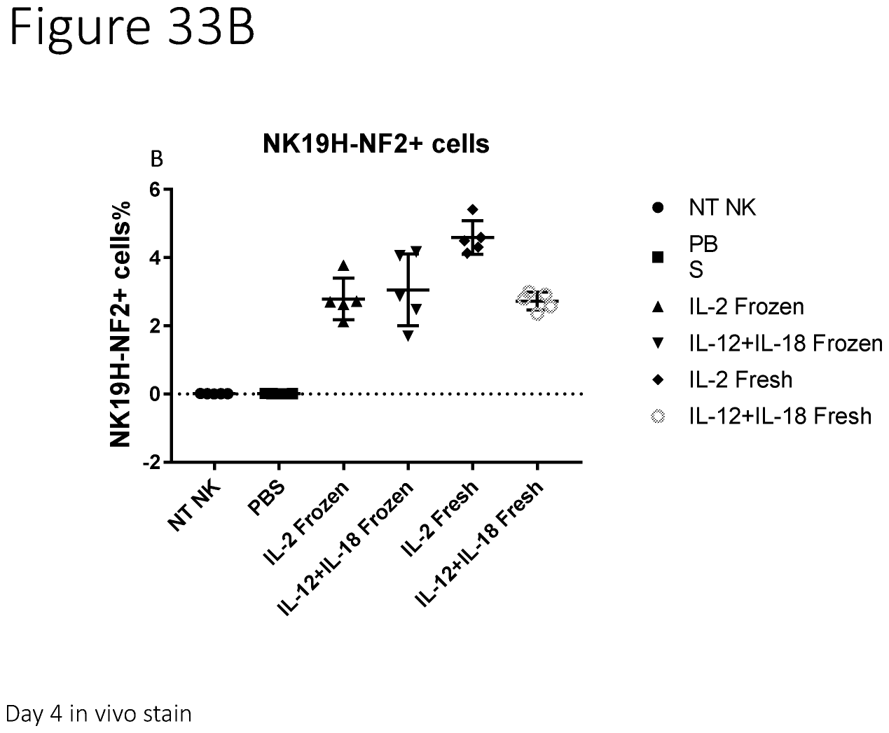

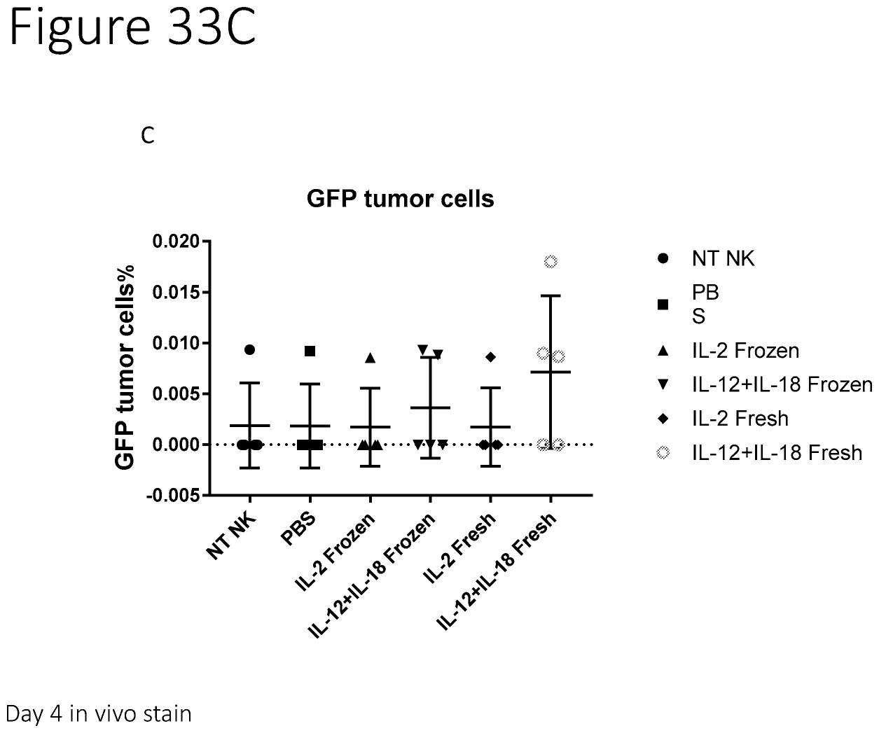

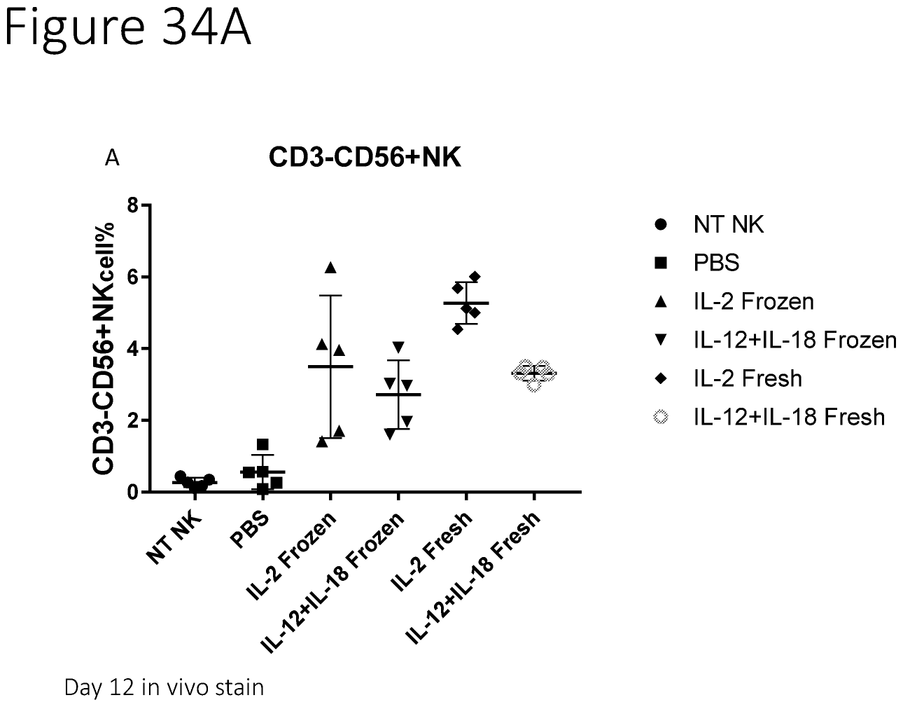

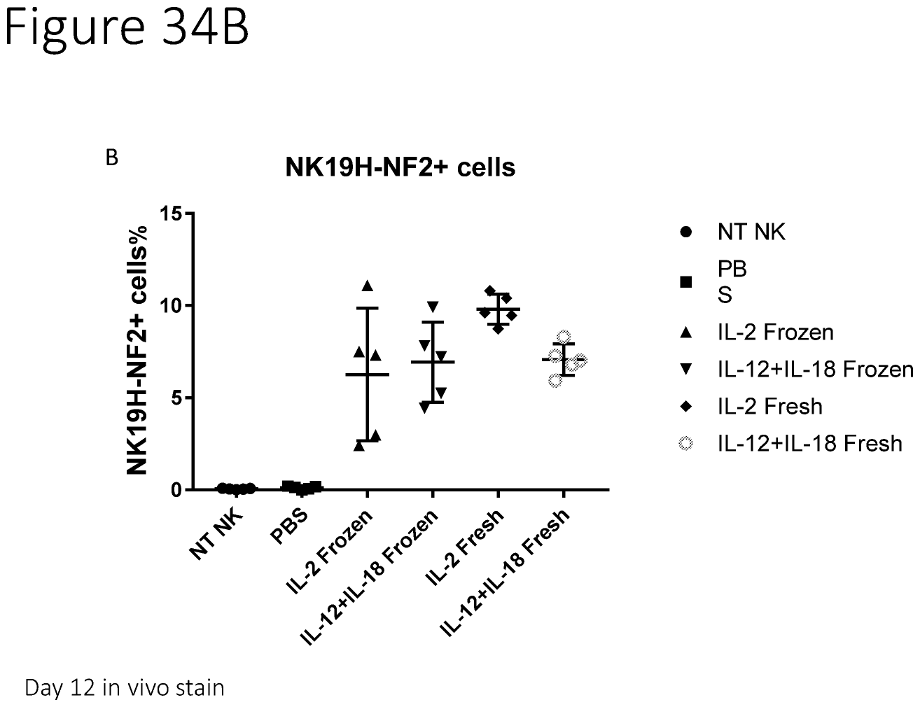

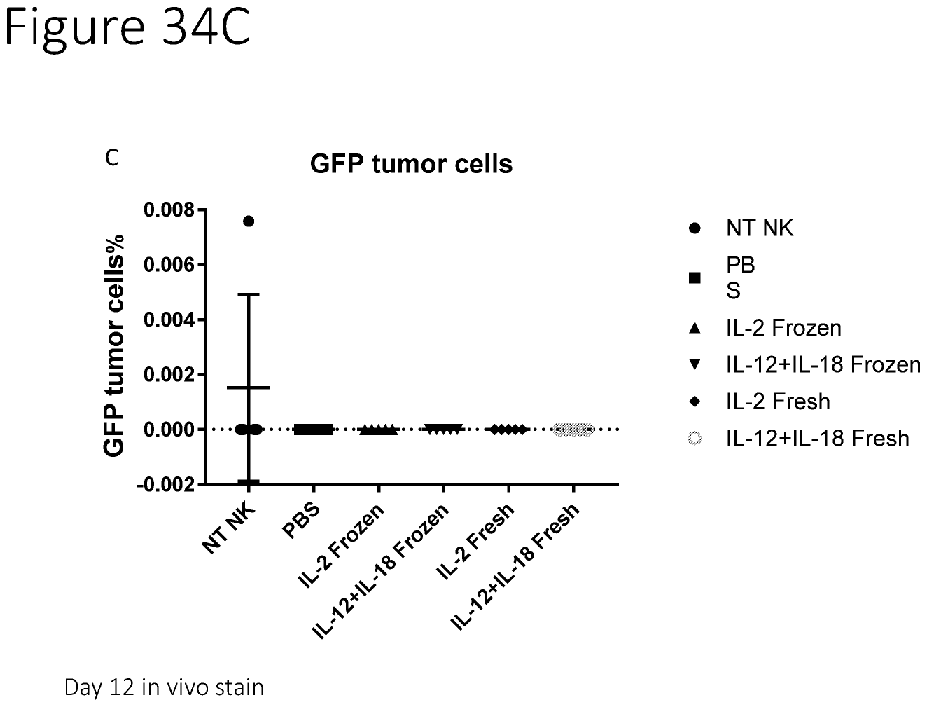

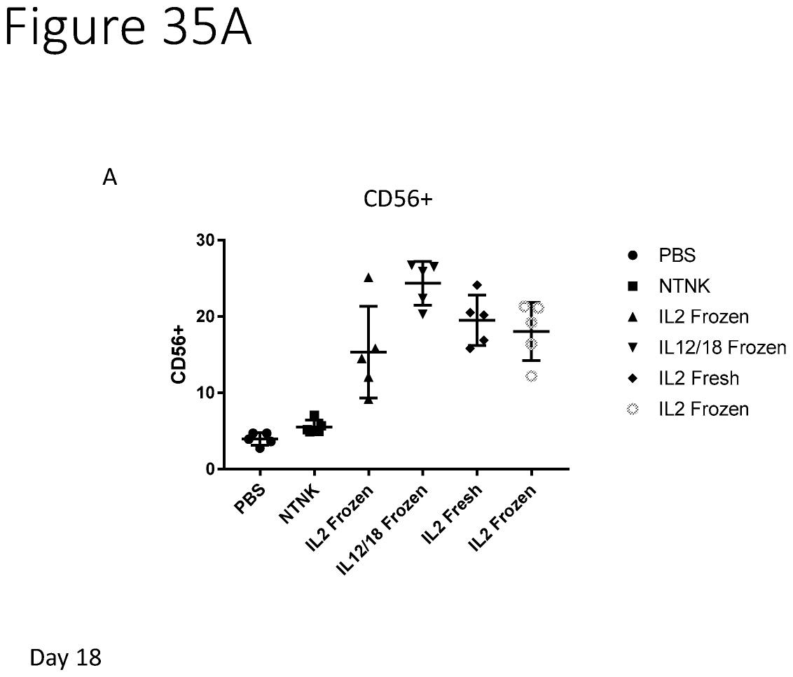

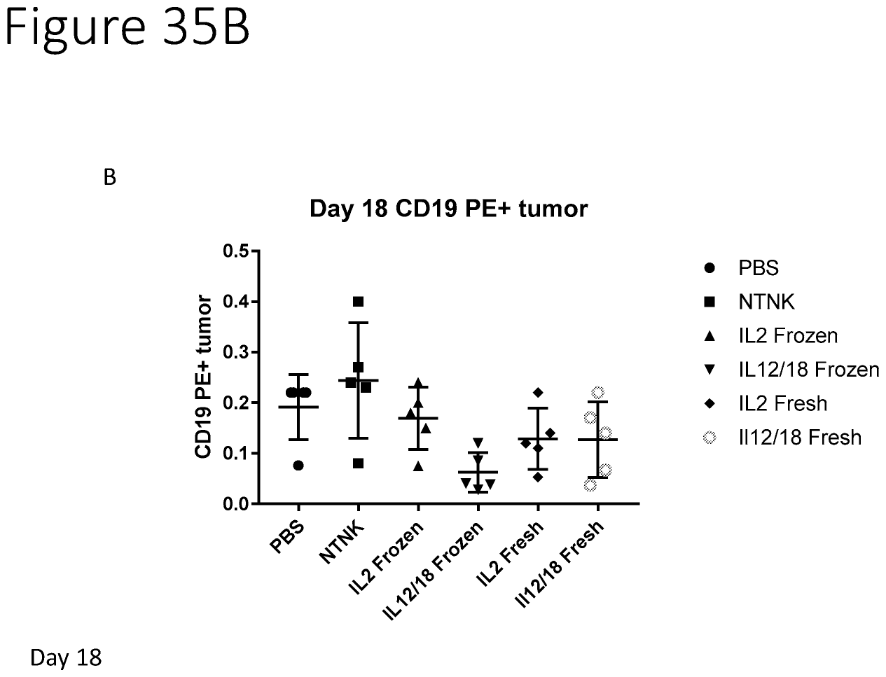

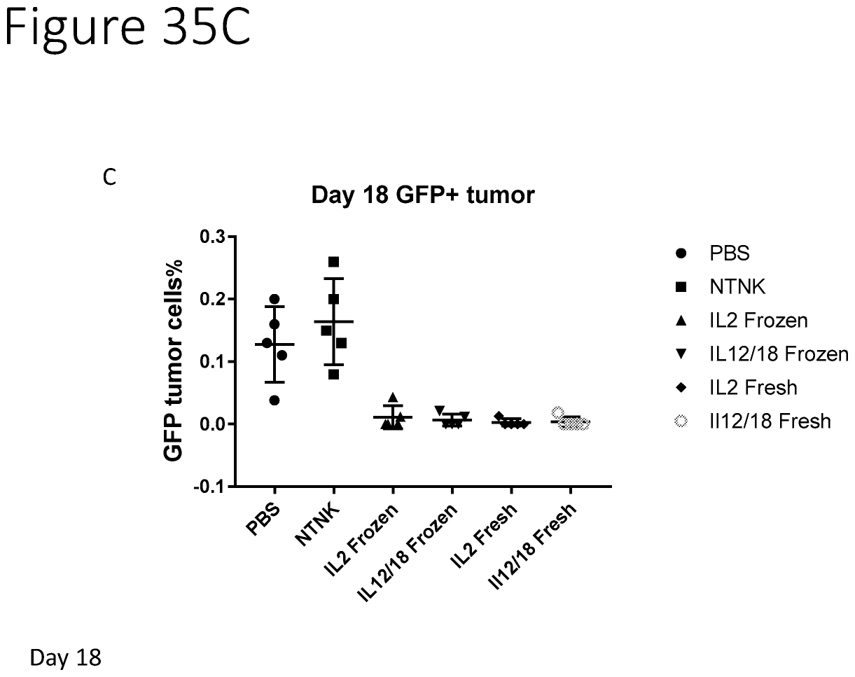

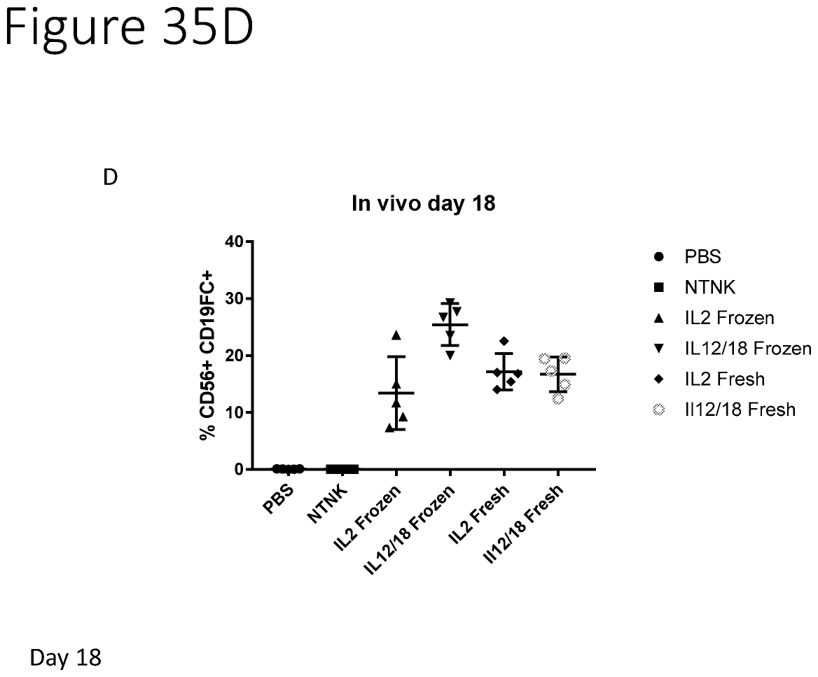

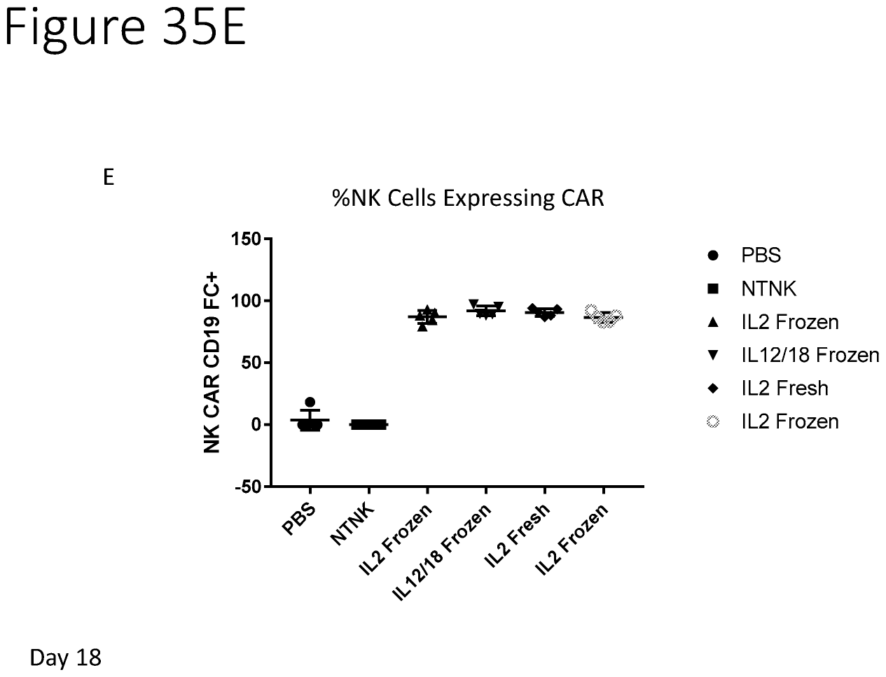

FIGS. 33, 34, and 35 relate to data further characterizing cells from the whole blood of animals at various time points post-tumor inoculation. FIG. 33 relates to data at day 4 post-administration, FIG. 34 relates to data at day 12 post-tumor inoculation, and FIG. 35 relates to data at day 18 post-tumor inoculation. These data relate to cells from the whole blood of animals treated as controls and receiving either non-transduced NK cells (NT NK) or PBS, or from one the other groups that received engineered NK cells expanded with IL2 in culture or IL12/18 in culture, with a fresh and frozen treatment group for each condition. FIG. 33A shows the percentage of NK cells (CD56-pos/CD3-neg) from whole blood of animals at Day 4. Each of the treatment groups were relatively similar in this regard, with about 3-5% of the cells in the whole blood being engineered NK cells. FIG. 33B shows data related to the percentage of cells that specifically express the non-limiting embodiment of an anti-CD19-CAR. Much like FIG. 33A, the percentage of anti-CD19-CAR-expressing cells in each of the treatment groups ranges from about 3-5%. FIG. 33C shows data related to the percentage of GFP-positive tumor cells present in the blood at day 4 post- administration. Consistent with the BLI imaging shown in prior figures, there is little detectable tumor cell presence in any treatment group. It may be that the low signal detected is reflective of the migration of the GFP+tumor cells from the circulation into various tissues (making them potentially detectable by BLI imaging but not in a blood sample per se). FIG. 34A-34C shows corresponding data 12 days after tumor inoculation. As was the case with the earlier time-point, each of the treatment groups result in between about 3%-5% of the blood cells in a sample were NK cells (FIG. 34A). FIG. 34B shows the percentage of cells positive for the anti-CD19 CAR construct. While the expression levels were similar across the treatment groups at this time-point, each experimental groups was present at levels notable above the control groups. Also, at 12 days, the percent of anti-CD19 expressing CAR cells (e.g., NK cells) was slightly higher (approximately 7-9% of the blood cells), suggesting an increased persistence of the engineered cells in the circulation. FIG. 34C shows the number of tumor cells in whole blood. Interestingly, all groups show little GFP expression, despite the BLI imaging showing increased luminescence, particularly in controls. Again, these data may reflect the physiological “residency” that certain suspension tumor cells exhibit.

FIG. 35A shows the percentage of NK cells (based on CD56-positivity) at 18 days after tumor inoculation. The experimental groups all show markedly higher percentages as compared to control groups, with the groups ranging from about 15% to about 25% of the cells in the whole blood. This increased percentage is consistent with the time window of increased NK cells as shown in FIG. 30B and 30C. While not statistically different in this particular experiment, these data show that NK cells expanded in IL12/IL18 media and cryopreserved were the most prolific of the experimental groups. According to several embodiments, the feeder cell plus cytokine-based expansion, coupled with cryopreservation yields a more robust NK cell that can survive under more normal cytokine conditions (e.g., without cytokine addiction) and can persist for longer periods of time in a health state. FIGS. 35B and 35C show two measures of tumor burden at day 18. FIG. 35B shows the percentage of cells in the blood that are positive for CD19 (the target of the engineered CAR in this non-limiting embodiment) as measured using an anti-CD19 PE-coupled antibody. These data show the trend upwards in the tumor burden in control groups, and in contrast, the ability of the engineered NK cells of the treatment groups to limit tumor growth. FIG. 35C shows similar data, but through the detection of GFP signal (e.g., ˜BLI). These data, while differing from those of 35B due to sensitivity of PE- versus GFP-based detection show a similar trend. The experimental NK cells show an enhanced ability to prevent the expansion of the tumor cells, as compared to controls. FIG. 35D relates to data regarding the number of NK cells that are expressing the engineered anti-CD19 (e.g., both CD56 and CD19 Fc positive). Similar to the data of FIG. 35A, these data show that an increased percentage of the NK cells in a blood sample are NK cells expressing the engineered anti-CD19 CAR, reflecting their enhanced persistence. FIG. 35E shows confirmatory data that nearly the entire population of NK cells of each experimental group that are positive for a CAR are NK cells that were engineered to express the anti-CD19 CAR disclosed herein.

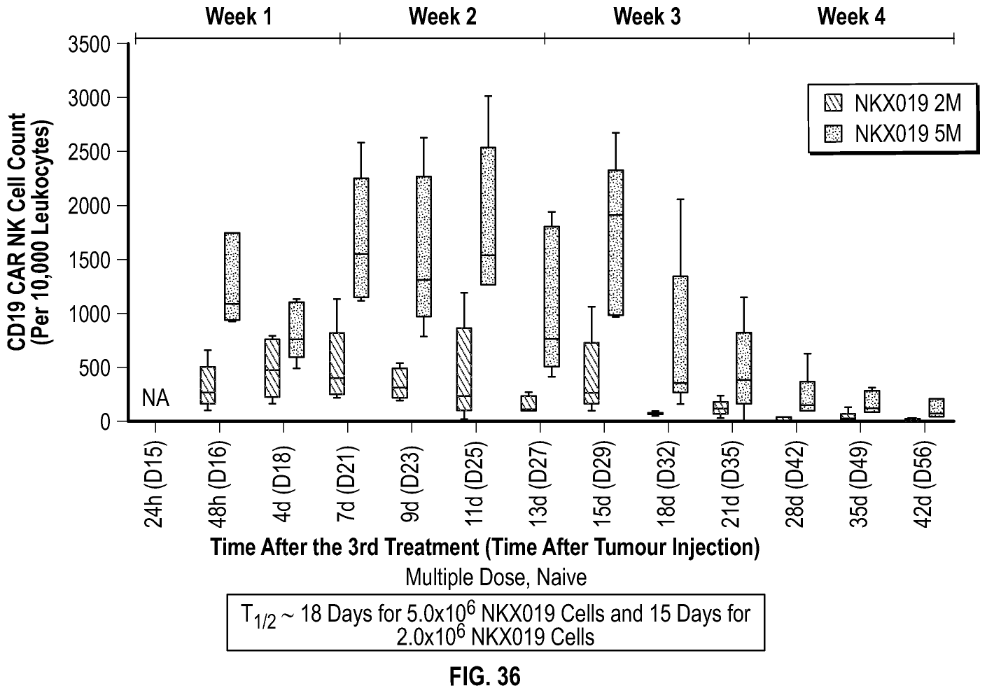

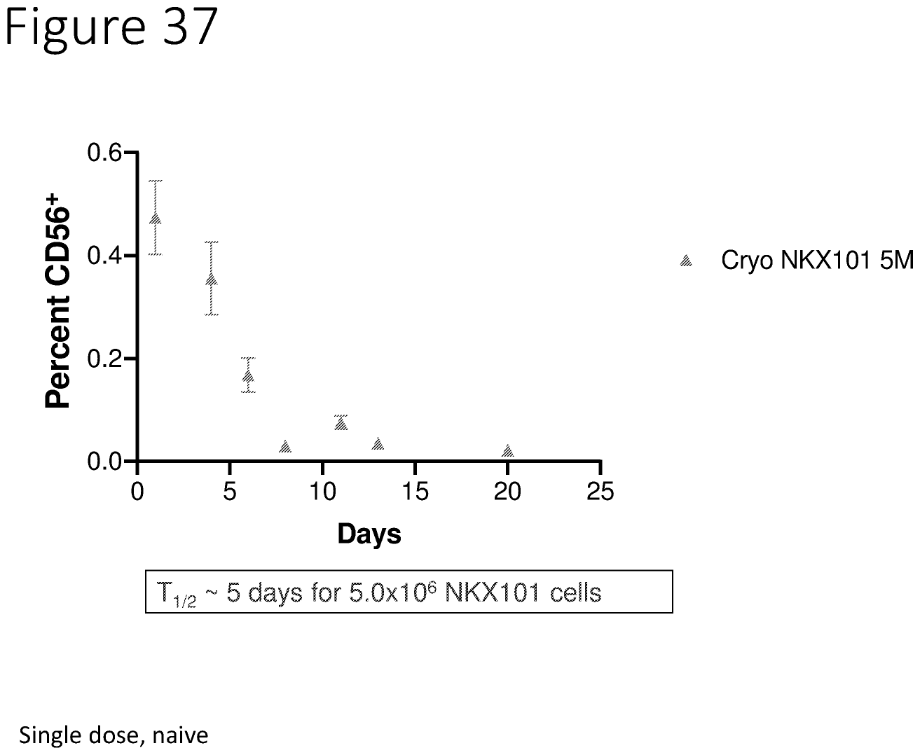

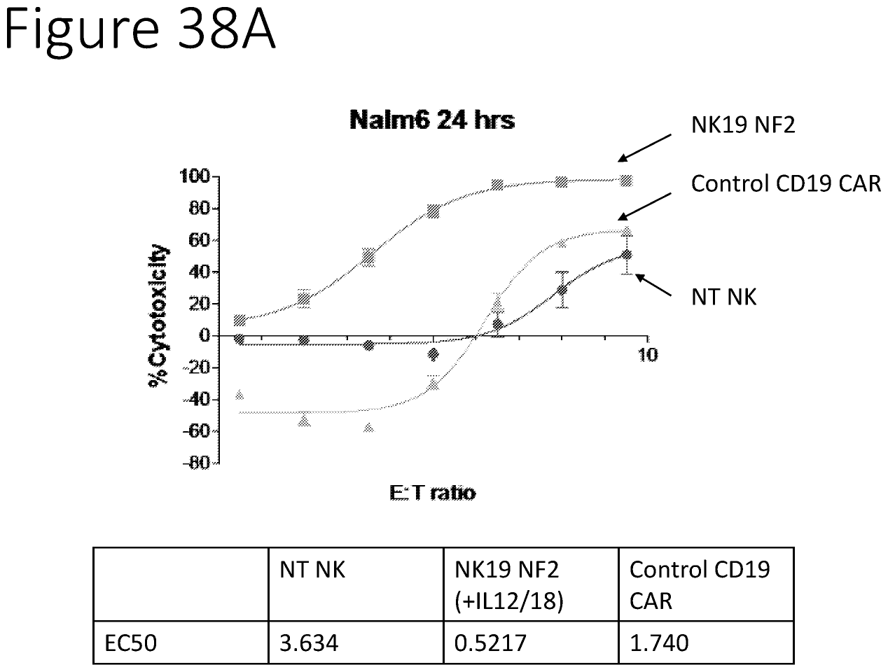

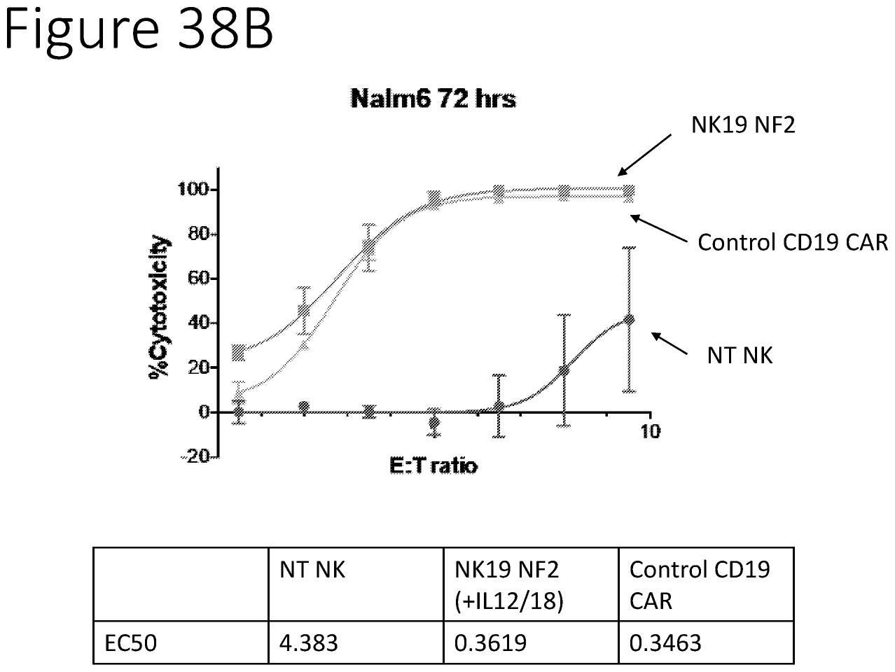

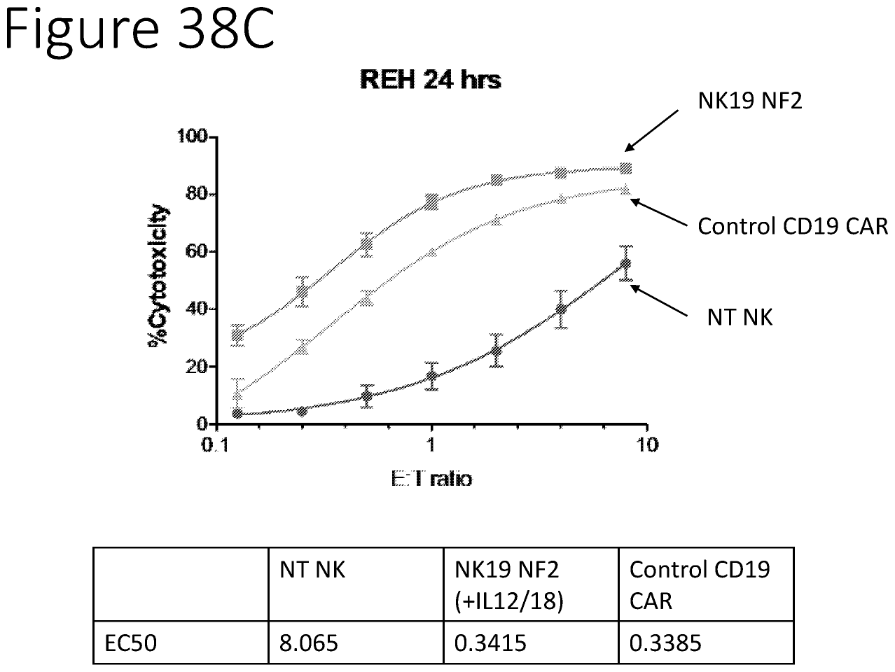

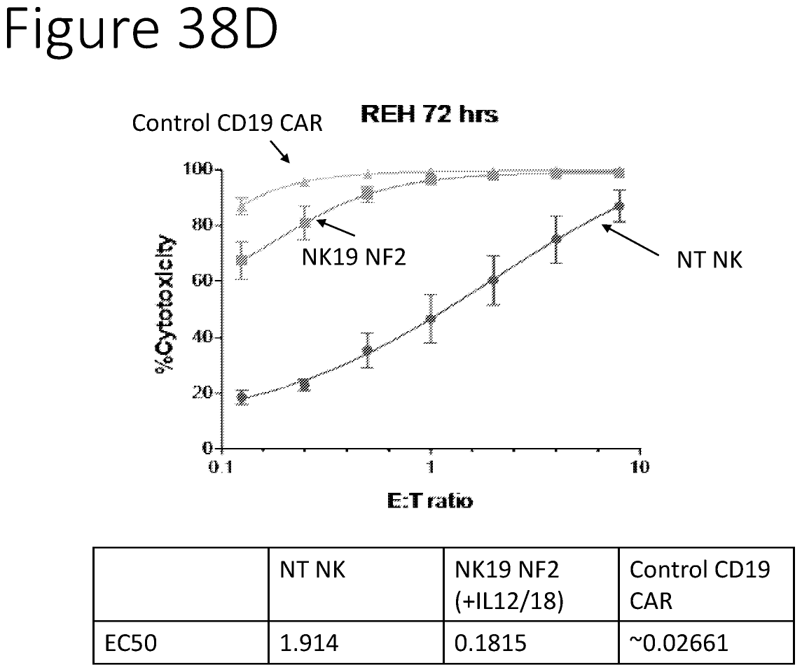

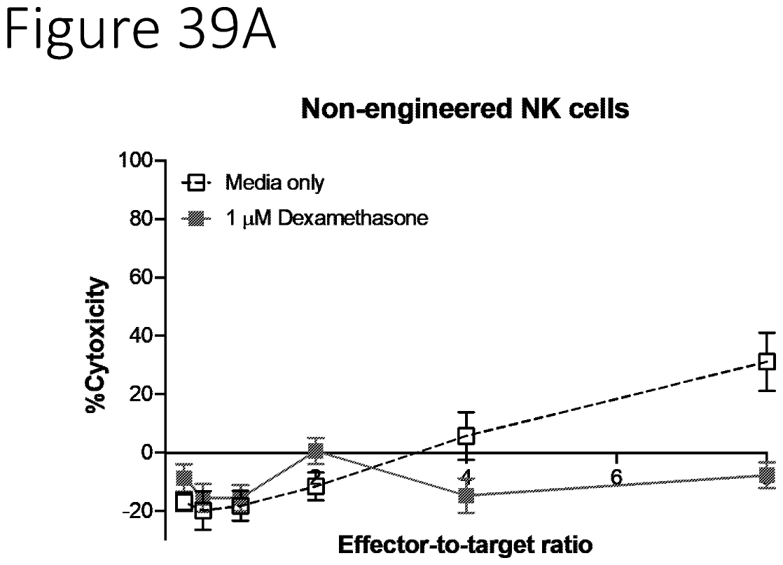

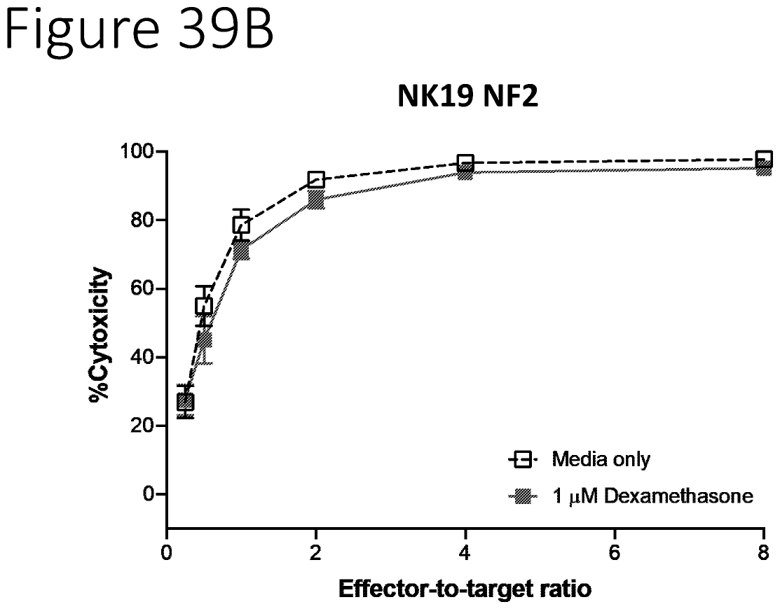

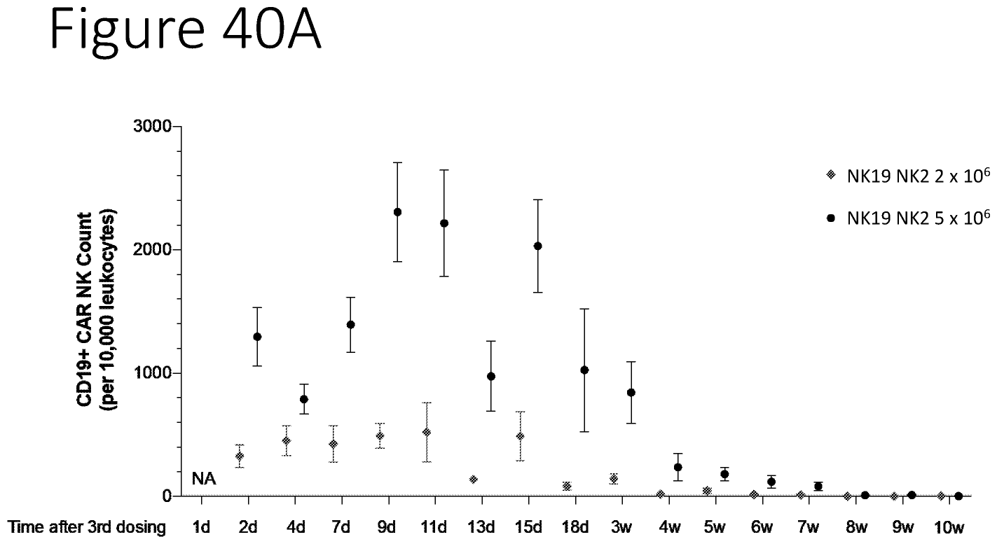

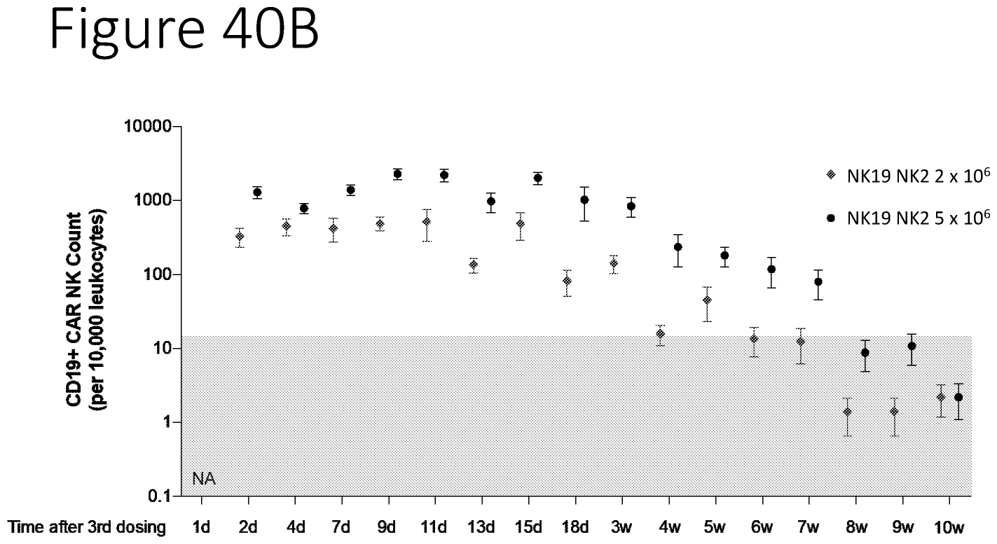

To further investigate the persistence of engineered NK cells expanded according to embodiments disclosed herein, two doses of engineered NK cells expanded using soluble cytokines as disclosed herein were administered to mice and cell numbers were tracked over four additional weeks (administration protocol per FIG. 27A). FIG. 36 shows a box plot of these data. In brief, the X axis of the box plot represents the time in two format, either: i) the time after the third administration or ii) total time since tumor inoculation (shows in parenthesis). The Y- axis represents the count of anti-CD19 CAR-expressing NK cells (per 10,000 leukocytes). The box plots for the 2 million NK cell dose are the lower trace of boxes (indicated by the dashed arrow), while the 5 million cell dose is the upper trace (indicated by the solid arrow). These data indicate that the half-life of engineered NK cells expanded in conditions where one or more stimulatory molecules (such as IL12 and/or IL18) are used (in conjunction with feeder cells, as described in several embodiments herein) is extended as compared to engineered NK cells expanded in feeder cell-only conditions. The half-life for a 2 million engineered NK cell dose is ˜15 days. Based on variance in one or more of clearance and/or volume of distribution, the half-life of a 5 million engineered NK cell dose is ˜18 days. These are in contrast to a dose of another engineered NK cell expanded without the use of the one or more additional stimulatory molecules, which is shown in FIG. 37, and indicates a half-life of ˜5 days for a dose of 5 million cells. Thus, according to several embodiments disclosed herein, the expansion of engineered NK cells using one or more additional cytokines, in conjunction with a feeder cell system, allows for the increased expansion of the NK cells and imparts to those cells an enhanced persistence and/or cytotoxicity.

### Example 5—Multiple-pulse Feeder Cells and Enhance NK Cell Expansion

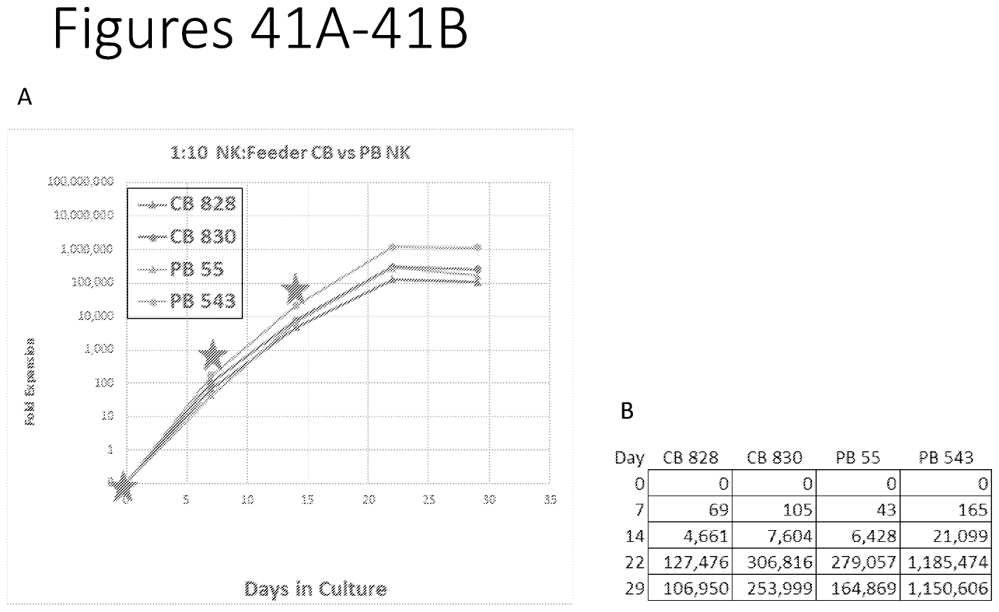

While embodiments disclosed herein result in the robust expansion of NK cells, additional embodiments were evaluated in order to determine if greater degrees of expansion could be achieved, while maintain the advantageous characteristics imparted to the expanded NK cells and/or minimizing deleterious effects or characteristics of the cells. NK cells were plated at a 1:10 ratio with feeder cells (here, K562 cells expressing membrane-bound IL15 and 4-1 BB ligand were used as a non-limiting example of a feeder cell) in media supplemented with 40 units/mL of IL2. NK cells were obtained from either cord blood or peripheral blood. The NK cells cultured for ˜3 weeks, starting at Day 0 and pulsed again with new feeder cells and media at day 7 and 14. Expanded cells were counted on Day 7, 14, 22 and Day 29. Results of expansion are shown graphically in FIG. 41A and summarized numerically in FIG. 41B. Each of the samples expanded reached a peak of cell number at Day 22 and maintained roughly the same number until Day 29. These data show cell expansion ranged from about 100,000-fold to over 1 million-fold (for NK cells from peripheral blood).

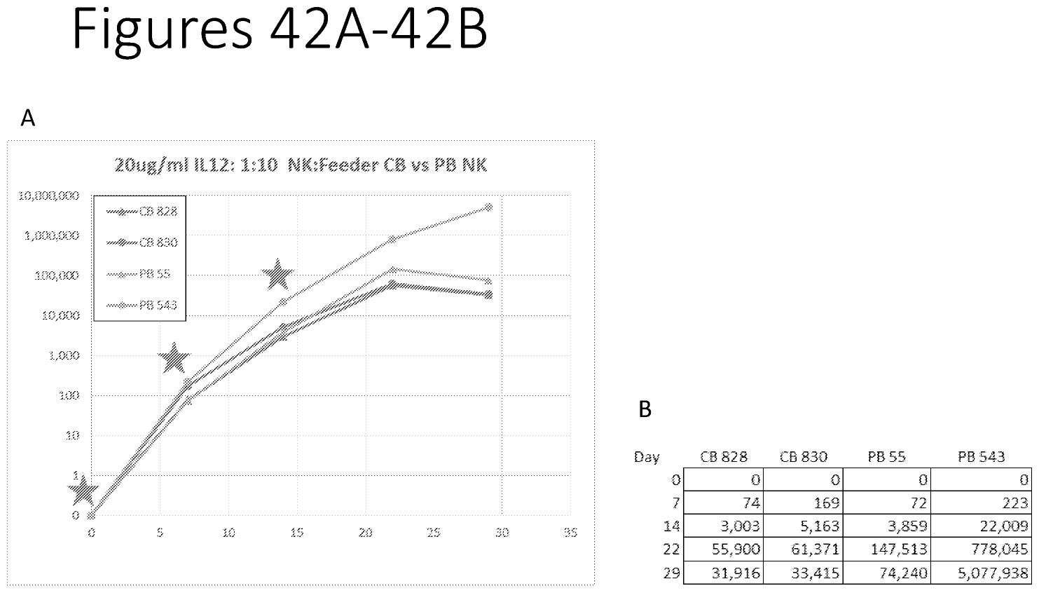

As discussed herein, in several embodiments, the culture media is supplemented with one or more of IL12 or IL18. Further information about such embodiments is disclosed in International PCT Patent Application No: PCT/US2020/044033, filed Jul. 29, 2020, which is incorporated in its entirety by reference herein. To investigate the effects of these when used in a multiple-pulse expansion format, media was supplemented with 40 IU/mL of IL2 and 20 ug/mL IL12 and used when NK cells from either cord blood or peripheral blood were pulsed at Day 0, 7, and 14, and counted at Day 7, 14, 22, and 29. Results of expansion are shown graphically in FIG. 42A and summarized numerically in FIG. 42B. Interestingly, cell expansion reached its peak at Day 22 in several samples, but peaked at Day 29 in peripheral blood from one donor. These data show that IL12 can promote the enhanced expansion of NK cells, in particular from peripheral blood.

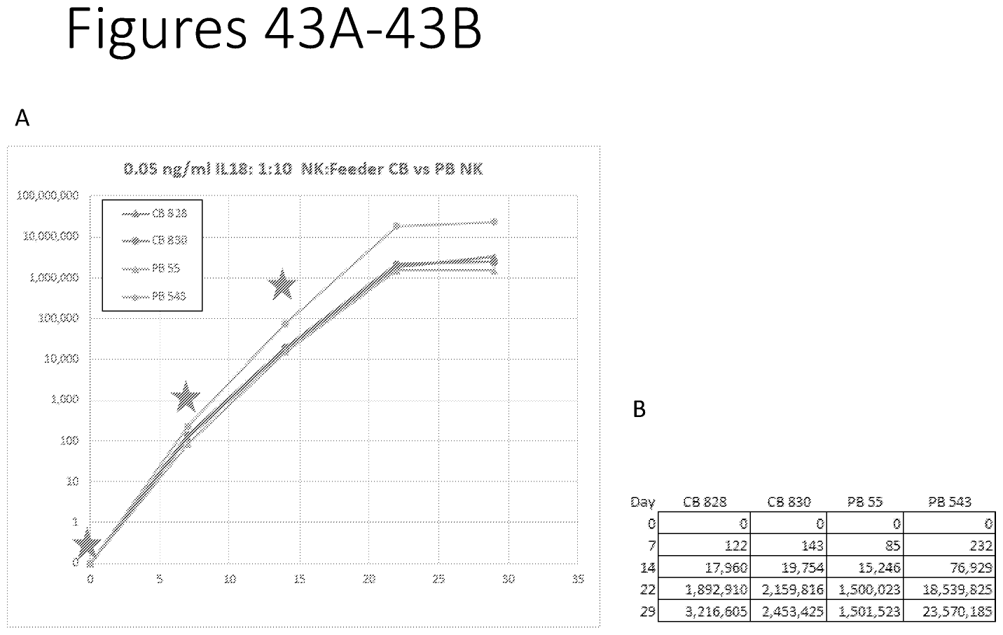

Similarly, the effects of IL18 were investigated by supplementing the culture media with 0.05 ng/mL IL18 (and 40 IU/mL IL2). Results of expansion are shown graphically in FIG. 43A and summarized numerically in FIG. 43B. These data show that IL18 can drive substantial cell expansion, both for cord blood and peripheral blood-derived NK cells. One donor showed over 20 million-fold increase in NK cells as a result of pulsing the initial NK cells at Day 7 and 14 (as well as at inception of culture).

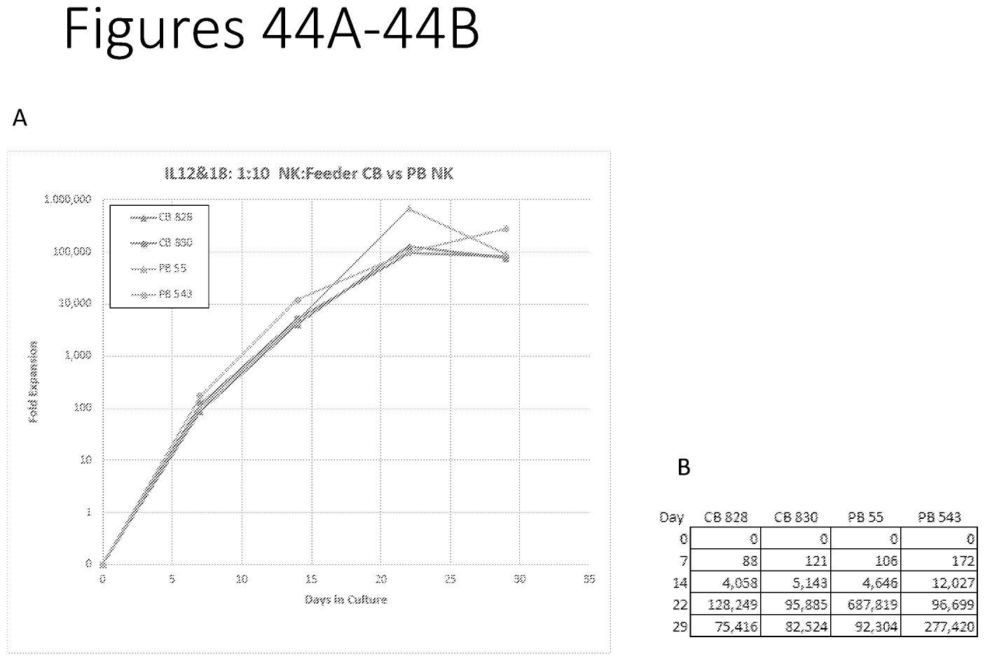

FIGS. 44A and 44B show the result of pulsing cord blood-derived or peripheral blood-derived NK cells with new feeder cells and media supplemented with both IL12 (20 ng/mL) and IL18 (0.05 ng/mL) (as well as 40 IU/mL of IL2). NK cells were pulsed at Day 7 and Day 14 (as well as at the inception of culture) and cells were counted on Day 7, 14, 22, and 29. Interestingly, these data indicated show that, with a 3 pulse approach, the combination of IL12 and IL18 supplemented media resulted in reduced cell expansion, perhaps suggesting that the cells were overstimulated or driven to expansion exhaustion by the repeated presentation of IL12, IL18 and IL2. This is in contrast to data shown in International PCT Patent Application No: PCT/US2020/044033, filed Jul. 29, 2020, which indicate that, according to some embodiments disclosed therein, addition of soluble IL12 and IL18 (but not in a 3-pulse fashion, as with this specific experiment) drive robust NK cell expansion.

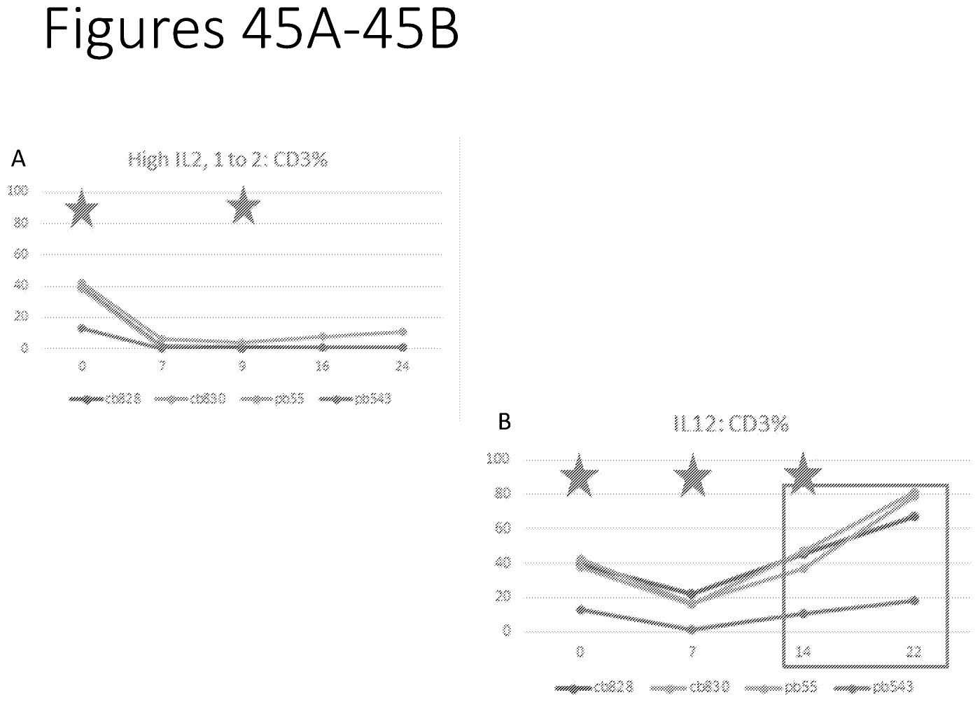

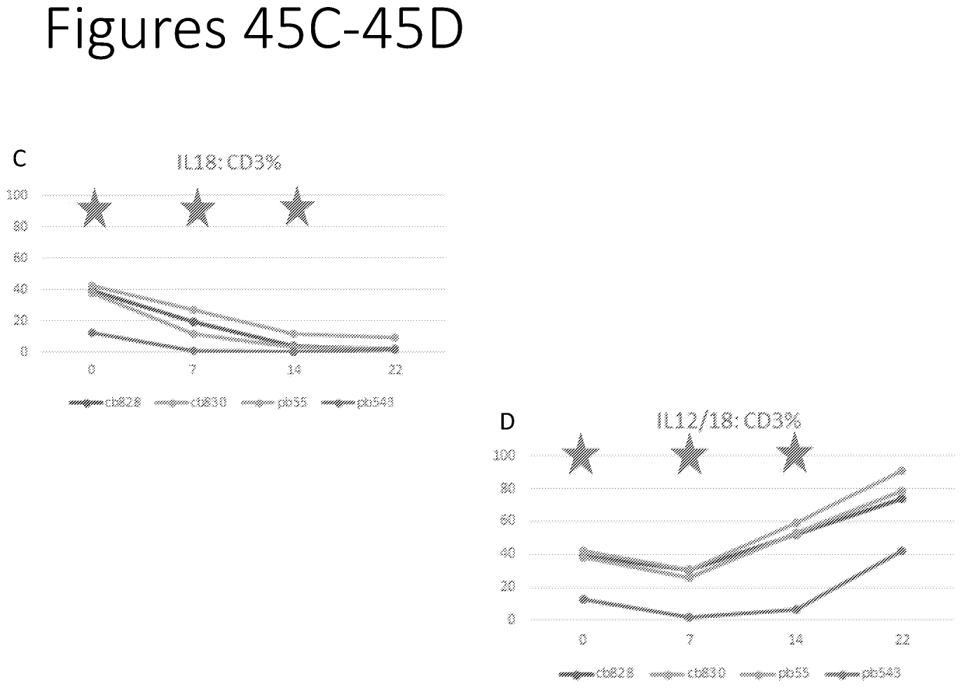

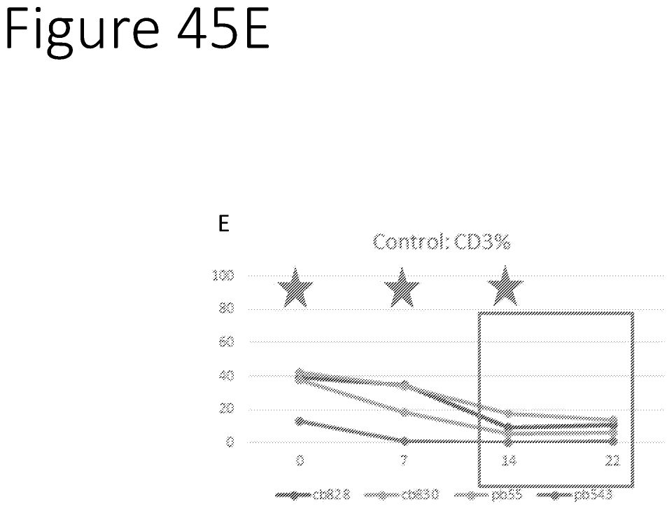

To further characterize the effects of multiple pulses of feeder cells and stimulatory cytokines on expanding immune cells, here NK cells, additional data was collected to assess the expansion of a CD3-positive subpopulation of cells. While, according to some embodiments, NK cells are purified (e.g., to remove T cells and other non-NK cells), there does remain some small residual T cell subpopulations. Alternatively, according to some embodiments, NK cells are not purified prior to expansion. Thus, under certain conditions, a T-cell subpopulation could result from the expansion of cells collected from a donor. This is evaluated in FIGS. 45A-45E. FIG. 45A shows limited expansion of a CD3-positive population when a starting population of blood cells (NK cells (in contrast to the starting material of FIGS. 45B-E were CBMC or PBMC)) was expanded in high concentrations of IL2 (data are presented as the percentage of cells that are CD3-positive). In contrast, FIG. 45B shows that use of IL12 in a 3-pulse expansion setting results in a greater degree of expansion of CD3-positive cells over time. Use of IL18 during a 3-pulse expansion did not increase the growth of the CD3-positive cells, as shown in FIG. 45C. Use of IL12 and IL18 in combination resulted in a similar increase in the CD3-postive subpopulation (see FIG. 45D), thus indicating that the presence of IL12, even in combination with IL18, can result in expansion of the CD3-positive subpopulation of cells (if such cells are present in the starting population of cells to be expanded), at least under certain conditions. FIG. 45E shows limited CD3-positive expansion under control conditions. Thus, in several embodiments, IL12 is used at concentrations that do not result in expansion of the CD3-positive subpopulation and or IL12 is not introduced into the media used in a given pulse at the same concentration as the prior pulse employed IL2. In several embodiments, IL12 is included in the media in fewer than the total number of pulses used during expansion, for example every other pulse, every third pulse, etc. In several embodiments, IL12 is present in a concentration of less than about 20 ng/mL, for example about 15 ng/mL, about 12 ng/mL, about 15 ng/mL, about 15 ng/mL, about 15 ng/mL, about 15 ng/mL, or any concentration between those listed. In several embodiments, a concentration of IL18 is used that offsets CD3-positive cell expansion, even if IL12 is used, for example about 0.07 ng/mL, about 0.10 ng/ml, about 0.12 ng/ml, about 0.15 ng/ml, about 0.2 ng/ml, or more. In some embodiments, despite CD3-postive cell expansion, the overall growth of the CD3-positive subset is offset by NK cell growth (e.g., CD56+/CD3− cells), such that the percentage of CD3-positive cells is negligible in the overall resulting expanded population. In several embodiments, if CD3-postive cells are detected post-expansion, they are removed, for example by solid phase affinity (e.g., Sepharose beads or another solid support bearing anti-CD3 antibodies).

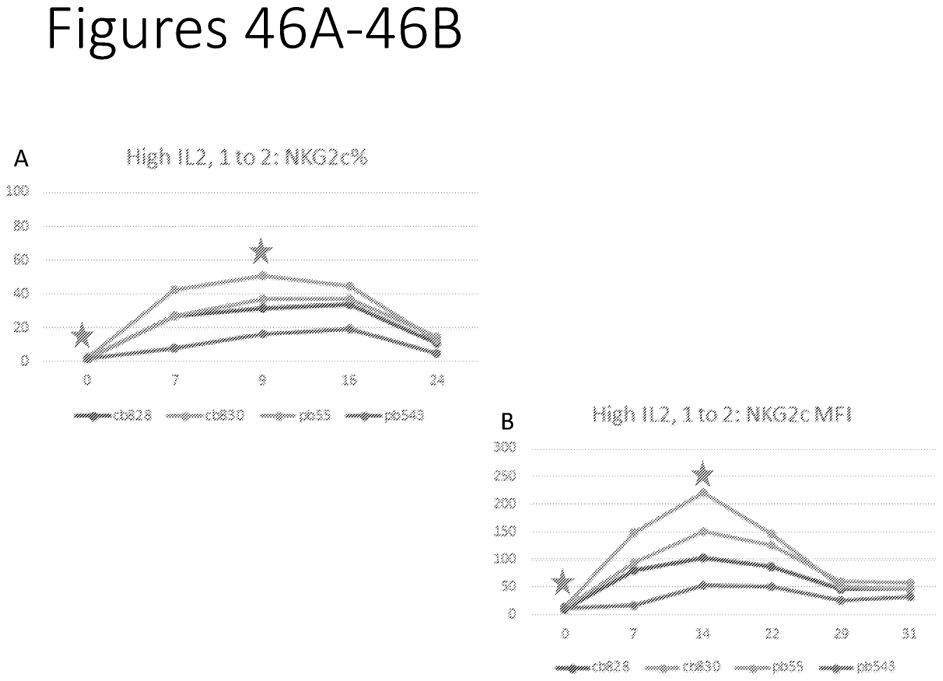

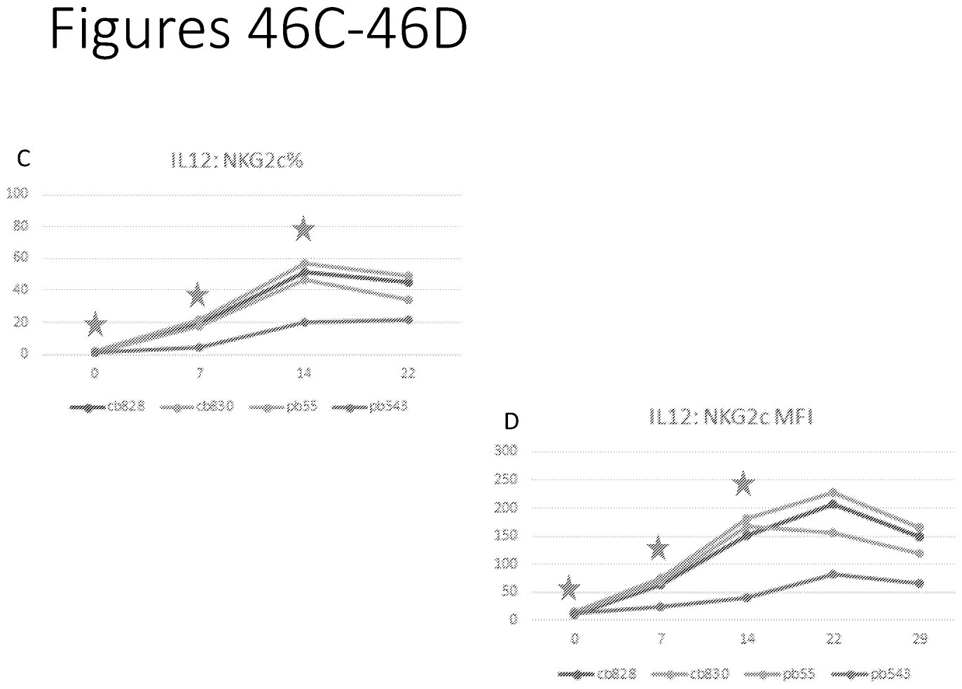

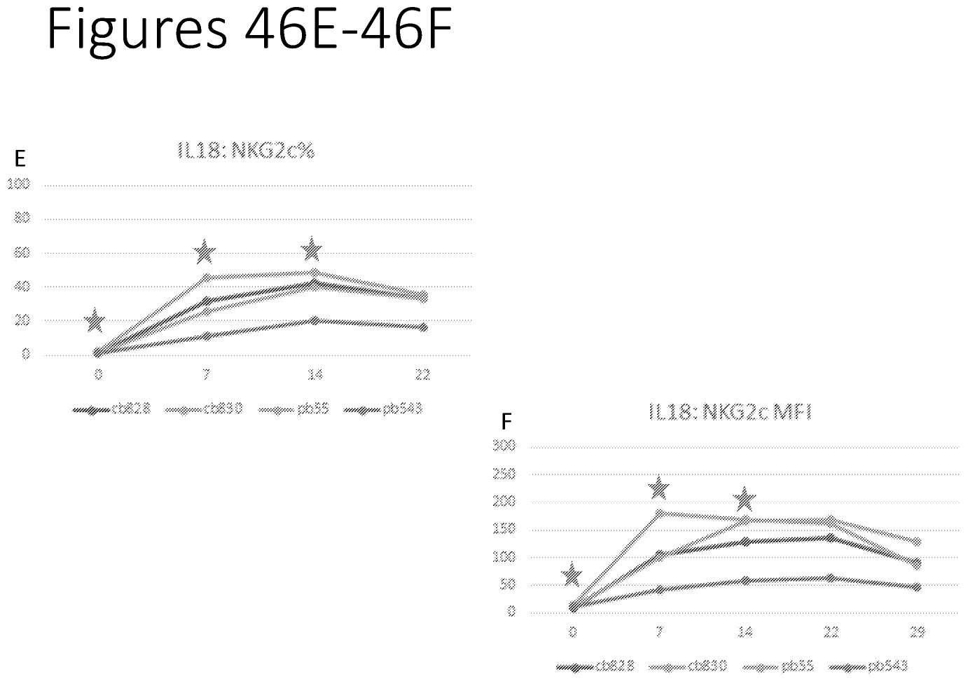

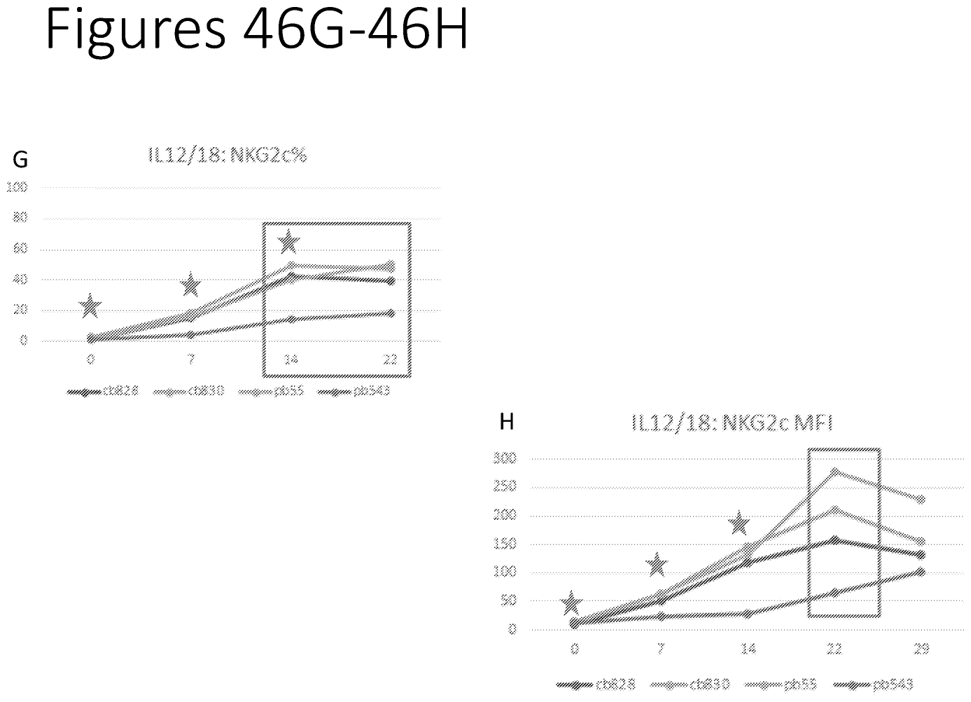

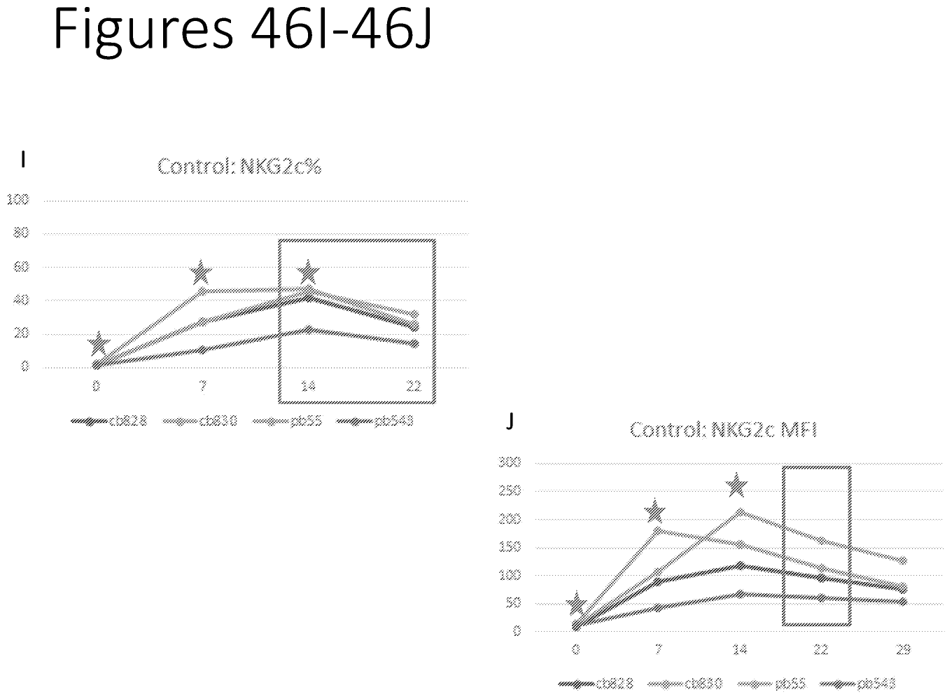

Additional data was collected relating to the degree of expression of the activating NKG2C receptor on NK cells expanded under the various conditions. FIG. 46A shows the percentage of NK cells expressing NKG2C over time when grown with high IL2, while FIG. 46B shows the same cells, but data expressed in terms of the overall MFI (which accounts for cells that express greater degrees of NKG2C which would not necessarily be demonstrated by the %-positive data). Use of IL12 in the culture media with three pulses resulted in a time-dependent increase in NKG2C expression through about 22 days (46C) with the overall expression by a given cell dropping thereafter (46D). IL18 in the media resulted in a similar profile of NKG2C expression, though with slightly enhanced stability of expression over time (FIGS. 46E and 46F). Use of IL12 and IL18 in combination with a three-pulse expansion resulted in steady increases in NKG2C expression over time, with the percentage of cells expressing NKG2C reaching similar levels as with either cytokine individually. The combination, however, did not result in a general decrease between days 14 and 22, as the cytokines individually caused. Moreover, as measured by the MFI, use of IL12 and IL18 together enhanced the “density” of NKG2C expression (FIG. 46H). Control NKG2C data are shown in FIGS. 46I and 46J).

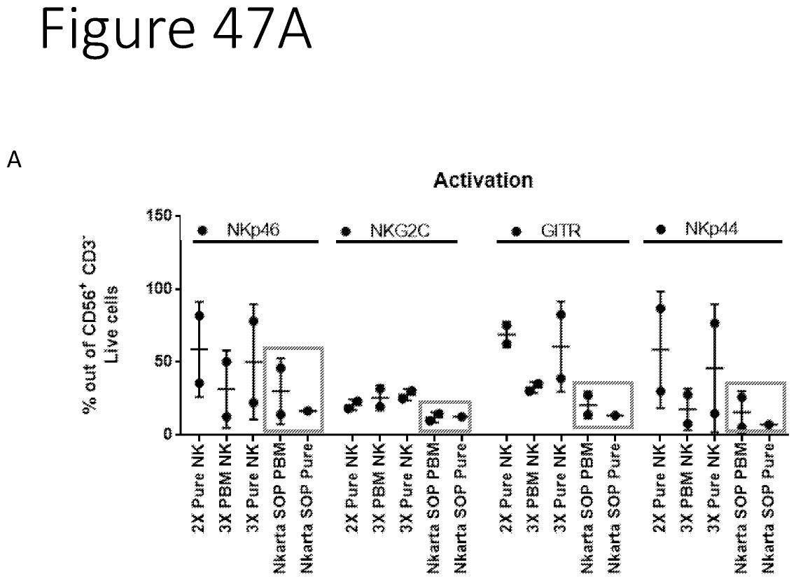

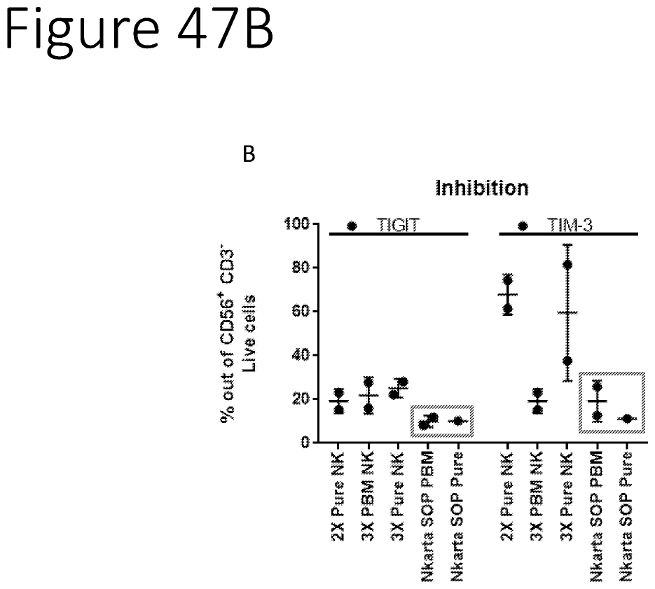

Further characterizing data was collected in relation to the expression of various markers (e.g., activating or inhibitory markers) by NK cells expanded under various conditions. FIG. 47A shows data indicating that multi-pulse expansion protocols yield an increase in the percentage of NK cells expressing markers of activation, such as increased expression of the natural cytotoxicity receptors NKp46 and NKp44, the NKG2C receptor and the Glucocorticoid-Induced Tumor Necrosis Factor Receptor (GITR). Interestingly, two markers of inhibition were also increased, the checkpoint receptor TIGIT and TIM3 (a protein domain involved in T cell tolerance), as shown in FIG. 47B.

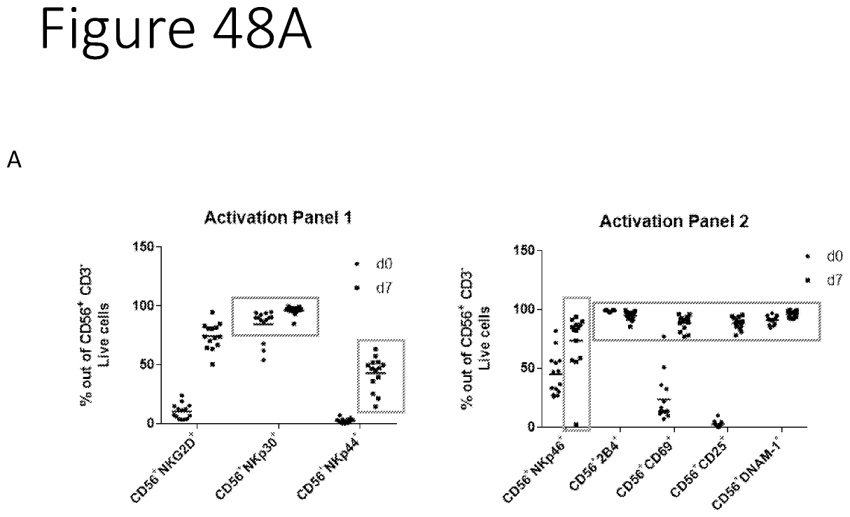

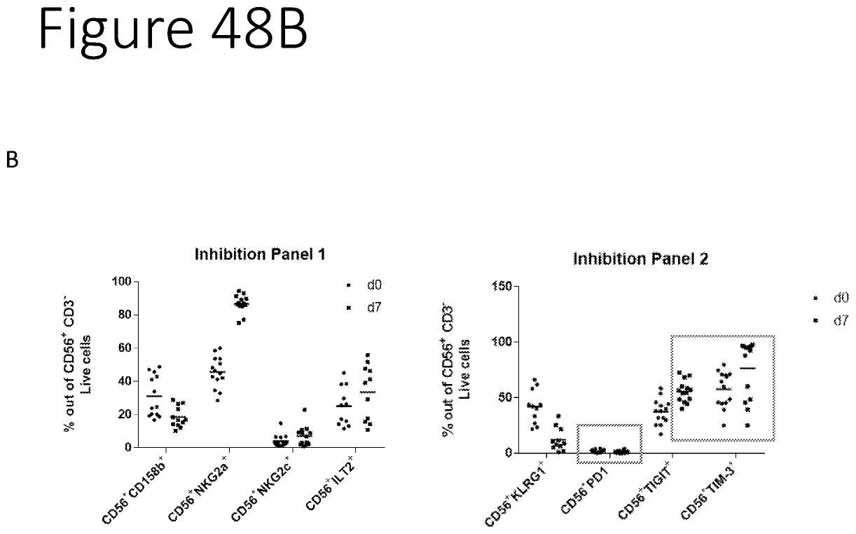

FIGS. 48A-48B relate to additional evaluation of the expression of activation (48A) or inhibitory (48B) markers at Day 0 (circles) or Day 7 (squares). FIG. 48A shows that, as discussed above, the natural cytotoxicity receptors NKp44 and NKp46 are expressed by more NK cells at Day 7 of the pulsed culture as compared to Day 0 (pulse 1). Other markers of activation, such as 2B4 expression, CD25 expression, are DNAM-1 expression remained consistently elevated during culture, with approximately 75% or more of the NK cells expressing those markers. FIG. 48B shows additional data the TIGIT and TIM3 are modestly increased over time in using the pulsed culture approach. However, in several embodiments, the multiple pulses used during expansion allow for production of clinically relevant NK cells that express characteristics of activated NK cells, thereby allowed their use in cancer immunotherapy.

It is contemplated that various combinations or sub-combinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “administering a population of expanded NK cells” includes “instructing the administration of a population of expanded NK cells.” In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “90%” includes “90%.” In some embodiments, at sequence having at least 95% sequence identity with a reference sequence includes sequences having 96%, 97%, 98%, 99%, or 100% identical to the reference sequence. In addition, when a sequence is disclosed as “comprising” a nucleotide or amino acid sequence, such a reference shall also include, unless otherwise indicated, that the sequence “comprises”, “consists of” or “consists essentially of” the recited sequence.

Articles such as “a”, “an”, “the” and the like, may mean one or more than one unless indicated to the contrary or otherwise evident from the context. The phrase “and/or” as used herein in the specification and in the claims, should be understood to mean “either or both” of the elements so conjoined. Multiple elements listed with “and/or” should be construed in the same fashion, i.e., “one or more” of the elements so conjoined. Other elements may optionally be present other than the elements specifically identified by the “and/or” clause. As used herein in the specification and in the claims, “or” should be understood to have the same meaning as “and/or” as defined above. For example, when used in a list of elements, “or” or “and/or” shall be interpreted as being inclusive, i.e., the inclusion of at least one, but optionally more than one, of list of elements, and, optionally, additional unlisted elements. Only terms clearly indicative to the contrary, such as “only one of” or “exactly one of” will refer to the inclusion of exactly one element of a number or list of elements. Thus claims that include “or” between one or more members of a group are considered satisfied if one, more than one, or all of the group members are present, employed in, or otherwise relevant to a given product or process unless indicated to the contrary. Embodiments are provided in which exactly one member of the group is present, employed in, or otherwise relevant to a given product or process. Embodiments are provided in which more than one, or all of the group members are present, employed in, or otherwise relevant to a given product or process. Any one or more claims may be amended to explicitly exclude any embodiment, aspect, feature, element, or characteristic, or any combination thereof. Any one or more claims may be amended to exclude any agent, composition, amount, dose, administration route, cell type, target, cellular marker, antigen, targeting moiety, or combination thereof.

In several embodiments, there are provided amino acid sequences that correspond to any of the nucleic acids disclosed herein, while accounting for degeneracy of the nucleic acid code. Furthermore, those sequences (whether nucleic acid or amino acid) that vary from those expressly disclosed herein, but have functional similarity or equivalency are also contemplated within the scope of the present disclosure. The foregoing includes mutants, truncations, substitutions, or other types of modifications.

Any titles or subheadings used herein are for organization purposes and should not be used to limit the scope of embodiments disclosed herein.

What is claimed is:

1. A method for enhancing the expansion of natural killer cells for use in immunotherapy, comprising:
co-culturing, in a culture media, a population of natural killer (NK) cells with a first population of feeder cells for a first period of time,
wherein the first feeder cell population comprises cells engineered to express 4-1 BBL and membrane-bound interleukin-15 (mbIL15),
wherein the population of NK cells comprises fewer cells than the population of feeder cells,
wherein the culture media comprises interleukin 2 (IL2), interleukin 12 (IL12), and interleukin 18 (IL18),
wherein the co-culturing for the first period of time results in an expanded population of NK cells;
separating, after the first period of time, at least a portion of the expanded population of NK cells from the feeder cells,
co-culturing, in fresh culture media, the at least a portion of the expanded population of NK cells with a second population of the feeder cells for a second period of time,
wherein the population of NK cells comprises fewer cells than the population of feeder cells,
wherein the culture media comprises interleukin 2 (IL2), interleukin 12 (IL12), and interleukin 18 (IL18),
wherein the co-culturing for the second period of time results in a further expanded population of NK cells; and,
optionally repeating the separating and co-culturing steps at least one additional time using fresh culture media comprising IL2, IL12, and IL18, thereby resulting in additional expansion of the further expanded population of NK cells.

2. The method of claim 1, wherein the repeated co-culturing of the expanded NK cells with an additional population of the feeder cells and fresh media results in enhanced NK cell expansion as compared to expanding NK cells with the feeder cells in the absence of the repeated co-culturing.

3. The method of claim 1, wherein the IL2 is present in the media at a concentration between about 10 units/mL and about 100 units/mL, wherein the IL12 is present in the media at a concentration between about 10 ng/mL and about 100 ng/mL, and wherein the IL18 is present in the media at a concentration between about 0.01 ng/mL and about 30 ng/mL, wherein the first and the second period of time are about 7 days, and wherein the co-culturing is repeated at least three times.

4. The method of claim 3, wherein the population of NK cells is present in an amount between about 5 and about 25 times less than the population of feeder cells at inception of each co-culturing.

5. The method of claim 1, wherein the expanded NK cells are separated from the feeder cells by Fluorescence-activated Cell Sorting (FACS).

6. The method of claim 1, wherein the feeder cell population comprises K562 cells that express both 4-1 BBL and mbIL15.

7. The method of claim 1, wherein the repeated co-culturing increases expression of markers of NK cell activation.

8. The method of claim 1, wherein the repeated co-culturing increases the cytotoxicity and/or persistence of the expanded NK cells.

9. The method of claim 1, further comprising contacting the NK cells with a vector encoding a chimeric antigen receptor (CAR).

10. The method of claim 9, wherein the CAR is configured to target one or more of CD19, CD123, CD70, BCMA, or a ligand of the natural killer receptor group D (NKG2D).

11. A method according to any one of claims 1 to 10, wherein the IL2 is present in the media at a concentration of less than about 50 units/mL, wherein the IL12 is present in the media at a concentration less than about 30 ng/mL, and wherein the IL18 is present in the media at a concentration of less than about 10 ng/mL.

12. A method according to any one of claims 1 to 10, wherein the IL2 is present in the media at a concentration between about 20 units/mL and about 50 units/mL, wherein the IL12 is present in the media at a concentration between about 15 ng/mL and about 30 ng/mL, and wherein the IL18 is present in the media at a concentration of less than about 5 ng/mL.

13. Use of the NK cells expanded by the method of any one of claims 1 to 12 for the preparation of a medicament for the treatment of cancer.

14. Use of the NK cells expanded by the method of any one of claims 1 to 12 for the treatment of cancer.

15. A population of engineered natural killer cells comprising,
an engineered chimeric receptor configured to bind a marker on a target cancer cell and upon binding, induce the NK cell to exert a cytotoxic effect against the target cancer cell,
wherein the NK cell was expanded by co-culturing for a first time, in a culture media comprising interleukin 2 (IL2), interleukin 12 (IL12), and interleukin 18 (IL18), a starting population of natural killer (NK) cells with a first population of feeder cells,
wherein the first population of feeder cells comprises cells engineered to express 4-1 BBL and membrane-bound interleukin-15 (mbIL15),
wherein the starting population of NK cells comprises fewer cells than the first population of feeder cells,
wherein the first co-culturing results in an intermediate expanded population of NK cells;
separating, after the first co-culturing, at least a portion of the intermediate expanded population of NK cells from the feeder cells,
co-culturing for at least as second time, in fresh culture media, at least a portion of the intermediate expanded population of NK cells with a second population of the feeder cells,
wherein the portion of the population of NK cells co-cultured with the second population of feeder cells comprises fewer cellsthan the second population of feeder cells,
wherein the at least a second co-culturing results in a further expanded population of NK cells.

16. The population of NK cells of claim 15, wherein the engineered chimeric receptor is encoded by a sequence at least 95% identical in sequence to one or more of SEQ ID NO: 1, 3, 5, 7, 9, 11, 13, 15, 17, 19, 21, 23, 25, or 27.

17. The population of NK cells of claim 15, wherein the engineered chimeric receptor has an amino acid sequence at least 95% identical in sequence to one or more of SEQ ID NO: 2, 4, 6, 8, 10, 12, 14, 16, 18, 20, 22, 24, 26, or 28.

18. Use of the NK cells of any one of claims 15 to 17 for the preparation of a medicament for the treatment of cancer.

19. Use of the NK cells expanded of any one of claims 15 to 17 for the treatment of cancer.

20. A method of treating cancer in a subject in need thereof, comprising administering to the subject a therapeutically effective amount of the engineered NK cells of any one of claims 15 to 17.

21. A method for enhancing the expansion of natural killer cells for use in immunotherapy, comprising:
co-culturing, in a culture media, a population of natural killer (NK) cells with a first population of feeder cells for a first period of time,
wherein the first feeder cell population comprises cells engineered to express 4-1 BBL and membrane-bound interleukin-15 (mbIL15),
wherein the population of NK cells comprises fewer cells than the population of feeder cells,
wherein the culture media comprises interleukin 2 (IL2), interleukin 12 (IL12), and interleukin 18 (IL18),
wherein the IL2 is present in the media in a concentration between about 0.5 ng/mL and about 6.0 ng/mL, wherein the IL12 is present in the media at a concentration between about 10 ng/mL and about 100 ng/mL, and wherein the IL18 is present in the media at a concentration between about 0.01 ng/mL and about 30 ng/mL,
wherein the co-culturing for the first period of time results in an expanded population of NK cells;
separating, after the first period of time, at least a portion of the expanded population of NK cells from the feeder cells,
co-culturing, in fresh culture media, the at least a portion of the expanded population of NK cells with a second population of the feeder cells for a second period of time,
wherein the population of NK cells comprises fewer cells than the population of feeder cells,
wherein the culture media comprises interleukin 2 (IL2), interleukin 12 (IL12), and interleukin 18 (IL18),
wherein the co-culturing for the second period of time results in a further expanded population of NK cells; and,
optionally repeating the separating and co-culturing steps at least one additional time using fresh culture media comprising IL2, IL12, and IL18, thereby resulting in additional expansion of the further expanded population of NK cells.

22. The method of claim 21, wherein the first and the second period of time are about 7 days, and wherein the co-culturing is repeated at least three times.
